SEQUENCE LISTING (339 sequences)

SEQ ID NO: 1 - protein, 128 aa, Artificial Sequence
  an artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCHYVHYASASTVLPAFGVDAWGQGTTVTVSS

SEQ ID NO: 2 - protein, 128 aa, Artificial Sequence
  an artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYVHYASASTLLPAFGVDAWGQGTTVTVSS

SEQ ID NO: 3 - protein, 128 aa, Artificial Sequence
  an artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYIHYASASTLLPAFGVDAWGQGTTVTVSS

SEQ ID NO: 4 - protein, 128 aa, Artificial Sequence
  an artificially synthesized sequence
QVQLVESGGGLVQPGRSLRLSCAASGFKFSNVWFHWVRQAPGKGLEWVAQIKDKYNAYATYYAPSVKGRFTISRDDSKNSIYLQMNSLKTEDTAVYYCHYVHYASASTLLPAFGVDAWGQGTTVTVSS

SEQ ID NO: 5 - protein, 128 aa, Artificial Sequence
  an artificially synthesized sequence
QVQLVESGGGLVQPGRSLRLSCAASGFVFSNVWMHWVRQAPGKGLEWVAQIKDKWNAYAAYYAPSVKGRFTISRDDSKNSIYLQMNSLKTEDTAVYYCHYIHYASASTLLPAEGIDAWGQGTTVTVSS

SEQ ID NO: 6 - protein, 128 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 7 - protein, 207 aa, Artificial Sequence
  an artificially synthesized sequence
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 8 - protein, 128 aa, Artificial Sequence
  an artificially synthesized sequence
QVQLVESGGGLVQPGRSLRLSCAASGFKFSNTWFHWVRQAPGKGLEWVAQIKDYYNDYAAYYAPSVKGRFTISRDDSKNSIYLQMNSLKTEDTAVYYCHYVHYASASTLLPAEGVDAWGQGTTVTVSS

SEQ ID NO: 9 - protein, 128 aa, Artificial Sequence
  an artificially synthesized sequence
QVQLVESGGGLVQPGRSLRLSCAASGFVFSNVWFHWVRQAPGKGLEWVAQIKDKYNAYADYYAPSVKERFTISRDDSKNSIYLQMNSLKTEDTAVYYCHYVHYASASTLLPAEGVDAWGQGTTVTVSS

SEQ ID NO: 10 - protein, 128 aa, Artificial Sequence
  an artificially synthesized sequence
QVQLVESGGGLVQPGRSLRLSCAASGFVFSNVWFHWVRQAPGKGLEWVAQIKDKYNAYADYYAPSVEGRFTISRDDSKNSIYLQMNSLKTEDTAVYYCHYVHYASASTLLPAEGVDAWGQGTTVTVSS

SEQ ID NO: 11 - protein, 128 aa, Artificial Sequence
  an artificially synthesized sequence
QVQLVESGGGLVQPGRSLRLSCAASGFVFSNVWFHWVRQAPGKGLEWVAQIKDYYNAYADYYAPSVEGRFTISRDDSKNSIYLQMNSLKTEDTAVYYCHYVHYASASTLLPAEGVDAWGQGTTVTVSS

SEQ ID NO: 12 - protein, 128 aa, Artificial Sequence
  an artificially synthesized sequence
QVQLVESGGGLVQPGRSLRLSCAASGFVFSNVWFHWVRQAPGKGLEWVAQIKDKWNAYADYYAPSVKERFTISRDDSKNSIYLQMNSLKTEDTAVYYCHYIHYASASTLLPAEGIDAWGQGTTVTVSS

SEQ ID NO: 13 - protein, 128 aa, Artificial Sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASTLLPAEGVDAWGQGTTVTVSS

SEQ ID NO: 14 - protein, 128 aa, Artificial Sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSS

SEQ ID NO: 15 - protein, 5 aa, Artificial Sequence
  an artificially synthesized sequence
NAWMH

SEQ ID NO: 16 - protein, 5 aa, Artificial Sequence
  an artificially synthesized sequence
NVWMH

SEQ ID NO: 17 - protein, 5 aa, Artificial Sequence
  an artificially synthesized sequence
NVWMH

SEQ ID NO: 18 - protein, 5 aa, Artificial Sequence
  an artificially synthesized sequence
NVWFH

SEQ ID NO: 19 - protein, 5 aa, Artificial Sequence
  an artificially synthesized sequence
NVWMH

SEQ ID NO: 20 - protein, 5 aa, Artificial Sequence
  an artificially synthesized sequence
NVWFH

SEQ ID NO: 21 - protein, 72 aa, Artificial Sequence
  an artificially synthesized sequence
SPCPPNSFSSAGGQRTCDICRQCKGVFRTRKECSSTSNAECDCTPGFHCLGAGCSMCEQDCKQGQELTKKGC

SEQ ID NO: 22 - protein, 5 aa, Artificial Sequence
  an artificially synthesized sequence
NTWFH

SEQ ID NO: 23 - protein, 5 aa, Artificial Sequence
  an artificially synthesized sequence
NVWFH

SEQ ID NO: 24 - protein, 5 aa, Artificial Sequence
  an artificially synthesized sequence
NVWFH

SEQ ID NO: 25 - protein, 5 aa, Artificial Sequence
  an artificially synthesized sequence
NVWFH

SEQ ID NO: 26 - protein, 5 aa, Artificial Sequence
  an artificially synthesized sequence
NVWFH

SEQ ID NO: 27 - protein, 5 aa, Artificial Sequence
  an artificially synthesized sequence
NVWFH

SEQ ID NO: 28 - protein, 5 aa, Artificial Sequence
  an artificially synthesized sequence
NVWFH

SEQ ID NO: 29 - protein, 19 aa, Artificial Sequence
  an artificially synthesized sequence
QIKDKGNAYAAYYAPSVKG

SEQ ID NO: 30 - protein, 19 aa, Artificial Sequence
  an artificially synthesized sequence
QIKDKYNAYAAYYAPSVKG

SEQ ID NO: 31 - protein, 19 aa, Artificial Sequence
  an artificially synthesized sequence
QIKDKYNAYAAYYAPSVKG

SEQ ID NO: 32 - protein, 19 aa, Artificial Sequence
  an artificially synthesized sequence
QIKDKYNAYATYYAPSVKG

SEQ ID NO: 33 - protein, 19 aa, Artificial Sequence
  an artificially synthesized sequence
QIKDKWNAYAAYYAPSVKG

SEQ ID NO: 34 - protein, 19 aa, Artificial Sequence
  an artificially synthesized sequence
QIKDYYNAYAAYYAPSVKG

SEQ ID NO: 35 - protein, 31 aa, Artificial Sequence
  an artificially synthesized sequence
DCTPGFHCLGAGCSMCEQDCKQGQELTKKGC

SEQ ID NO: 36 - protein, 19 aa, Artificial Sequence
  an artificially synthesized sequence
QIKDYYNDYAAYYAPSVKG

SEQ ID NO: 37 - protein, 19 aa, Artificial Sequence
  an artificially synthesized sequence
QIKDKYNAYADYYAPSVKE

SEQ ID NO: 38 - protein, 19 aa, Artificial Sequence
  an artificially synthesized sequence
QIKDKYNAYADYYAPSVEG

SEQ ID NO: 39 - protein, 19 aa, Artificial Sequence
  an artificially synthesized sequence
QIKDYYNAYADYYAPSVEG

SEQ ID NO: 40 - protein, 19 aa, Artificial Sequence
  an artificially synthesized sequence
QIKDKWNAYADYYAPSVKE

SEQ ID NO: 41 - protein, 19 aa, Artificial Sequence
  an artificially synthesized sequence
QIKDYYNAYAGYYHPSVKG

SEQ ID NO: 42 - protein, 19 aa, Artificial Sequence
  an artificially synthesized sequence
QIKDYYNAYAGYYHPSVKG

SEQ ID NO: 43 - protein, 17 aa, Artificial Sequence
  an artificially synthesized sequence
VHYASASTVLPAFGVDA

SEQ ID NO: 44 - protein, 17 aa, Artificial Sequence
  an artificially synthesized sequence
VHYASASTLLPAFGVDA

SEQ ID NO: 45 - protein, 17 aa, Artificial Sequence
  an artificially synthesized sequence
IHYASASTLLPAFGVDA

SEQ ID NO: 46 - protein, 17 aa, Artificial Sequence
  an artificially synthesized sequence
VHYASASTLLPAFGVDA

SEQ ID NO: 47 - protein, 17 aa, Artificial Sequence
  an artificially synthesized sequence
IHYASASTLLPAEGIDA

SEQ ID NO: 48 - protein, 17 aa, Artificial Sequence
  an artificially synthesized sequence
VHYASASTLLPAEGVDA

SEQ ID NO: 49 - protein, 63 aa, Artificial Sequence
  an artificially synthesized sequence
LQDPCSNCPAGTFCDNNRNQICSPCPPNSFSSAGGQRTCDICRQCKGVFRTRKECSSTSNAEC

SEQ ID NO: 50 - protein, 17 aa, Artificial Sequence
  an artificially synthesized sequence
VHYASASTLLPAEGVDA

SEQ ID NO: 51 - protein, 17 aa, Artificial Sequence
  an artificially synthesized sequence
VHYASASTLLPAEGVDA

SEQ ID NO: 52 - protein, 17 aa, Artificial Sequence
  an artificially synthesized sequence
VHYASASTLLPAEGVDA

SEQ ID NO: 53 - protein, 17 aa, Artificial Sequence
  an artificially synthesized sequence
VHYASASTLLPAEGVDA

SEQ ID NO: 54 - protein, 17 aa, Artificial Sequence
  an artificially synthesized sequence
IHYASASTLLPAEGIDA

SEQ ID NO: 55 - protein, 17 aa, Artificial Sequence
  an artificially synthesized sequence
VHYAAASTLLPAEGVDA

SEQ ID NO: 56 - protein, 17 aa, Artificial Sequence
  an artificially synthesized sequence
VHYAAASQLLPAEGVDA

SEQ ID NO: 57 - protein, 112 aa, Artificial Sequence
  an artificially synthesized sequence
DIVMTQSPLSLPVTPGEPASISCQASQELVHMNRNTYLHWYQQKPGQAPRLLIYKVSNRFPGVPDRFSGSGSGTDFTLKISRVEAEDVGVYYCAQGTSVPFTFGQGTKLEIK

SEQ ID NO: 58 - protein, 112 aa, Artificial Sequence
  an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIK

SEQ ID NO: 59 - protein, 112 aa, Artificial Sequence
  an artificially synthesized sequence
DIVMTQSPLSLPVTPGEPASISCQPSQEVVHMNNVVYLHWYQQKPGQAPRLLIYKVSNRFPGVPDRFSGSGSGTDFTLKISRVEAEDVGVYYCAQGTSHPFTFGQGTKLEIK

SEQ ID NO: 60 - protein, 112 aa, Artificial Sequence
  an artificially synthesized sequence
DIVMTQSPLSLPVTPGEPASISCQPSQEVVHMNRNTYLHWYQQKPGQAPRLLIYKVSNVFPGVPDRFSGSGSGTDFTLKISRVEAEDVGVYYCAQGTHHPFTFGQGTKLEIK

SEQ ID NO: 61 - protein, 112 aa, Artificial Sequence
  an artificially synthesized sequence
DIVMTQSPLSLPVTPGEPASISCQPSEEVVHMNRNTYLHWYQQKPGQAPRLLIYKVSNLFPGVPDRFSGSGSGTDFTLKISRVEAEDVGVYYCAQGTHHPFTFGQGTKLEIK

SEQ ID NO: 62 - protein, 16 aa, Artificial Sequence
  an artificially synthesized sequence
QASQELVHMNRNTYLH

SEQ ID NO: 63 - protein, 16 aa, Artificial Sequence
  an artificially synthesized sequence
QPSQEVVHMNRNTYLH

SEQ ID NO: 64 - protein, 16 aa, Artificial Sequence
  an artificially synthesized sequence
QPSQEVVHMNNVVYLH

SEQ ID NO: 65 - protein, 16 aa, Artificial Sequence
  an artificially synthesized sequence
QPSQEVVHMNRNTYLH

SEQ ID NO: 66 - protein, 16 aa, Artificial Sequence
  an artificially synthesized sequence
QPSEEVVHMNRNTYLH

SEQ ID NO: 67 - protein, 7 aa, Artificial Sequence
  an artificially synthesized sequence
KVSNRFP

SEQ ID NO: 68 - protein, 7 aa, Artificial Sequence
  an artificially synthesized sequence
KVSNRFP

SEQ ID NO: 69 - protein, 7 aa, Artificial Sequence
  an artificially synthesized sequence
KVSNRFP

SEQ ID NO: 70 - protein, 7 aa, Artificial Sequence
  an artificially synthesized sequence
KVSNVFP

SEQ ID NO: 71 - protein, 7 aa, Artificial Sequence
  an artificially synthesized sequence
KVSNLFP

SEQ ID NO: 72 - protein, 9 aa, Artificial Sequence
  an artificially synthesized sequence
AQGTSVPFT

SEQ ID NO: 73 - protein, 9 aa, Artificial Sequence
  an artificially synthesized sequence
AQGTSHPFT

SEQ ID NO: 74 - protein, 9 aa, Artificial Sequence
  an artificially synthesized sequence
AQGTSHPFT

SEQ ID NO: 75 - protein, 9 aa, Artificial Sequence
  an artificially synthesized sequence
AQGTHHPFT

SEQ ID NO: 76 - protein, 9 aa, Artificial Sequence
  an artificially synthesized sequence
AQGTHHPFT

SEQ ID NO: 77 - protein, 122 aa, Artificial Sequence
  an artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 78 - protein, 112 aa, Artificial Sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 79 - protein, 121 aa, Artificial Sequence [sequence omitted]

SEQ ID NO: 80 - protein, 109 aa, Artificial Sequence [sequence omitted]

SEQ ID NO: 81 - protein, 128 aa, Artificial Sequence
  an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 82 - protein, 5 aa, Artificial Sequence
  an artificially synthesized sequence
NVWFH

SEQ ID NO: 83 - protein, 19 aa, Artificial Sequence
  an artificially synthesized sequence
QIKDYYNAYAAYYAPSVKG

SEQ ID NO: 84 - protein, 17 aa, Artificial Sequence
  an artificially synthesized sequence
VHYASASTLLPAEGVDA

SEQ ID NO: 85 - protein, 330 aa, Artificial Sequence [sequence omitted]

SEQ ID NO: 86 - protein, 326 aa, Artificial Sequence
  an artificially synthesized sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 377 aa, Artificial Sequence
  an artificially synthesized sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 88 - protein, 327 aa, Artificial Sequence
  an artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 88 aa, Artificial Sequence
  an artificially synthesized sequence
MVLASSTTSIHTMLLLLLMLAQPAMARADPCAARPCAHGGRCYAHFSGLVCACAPGYMGARCEFPVHPDGASALPAAPPGLRPGDPQR

SEQ ID NO: 90 - protein, 618 aa, Artificial Sequence
  an artificially synthesized sequence
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 91 - protein, 4 aa, Artificial Sequence
  an artificially synthesized sequence
GGGS

SEQ ID NO: 92 - protein, 4 aa, Artificial Sequence
  an artificially synthesized sequence
SGGG

SEQ ID NO: 93 - protein, 5 aa, Artificial Sequence
  an artificially synthesized sequence
GGGGS

SEQ ID NO: 94 - protein, 5 aa, Artificial Sequence
  an artificially synthesized sequence
SGGGG

SEQ ID NO: 95 - protein, 6 aa, Artificial Sequence
  an artificially synthesized sequence
GGGGGS

SEQ ID NO: 96 - protein, 6 aa, Artificial Sequence
  an artificially synthesized sequence
SGGGGG

SEQ ID NO: 97 - protein, 7 aa, Artificial Sequence
  an artificially synthesized sequence
GGGGGGS

SEQ ID NO: 98 - protein, 7 aa, Artificial Sequence
  an artificially synthesized sequence
SGGGGGG

SEQ ID NO: 99 - protein, 220 aa, Artificial Sequence
  an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 100 - protein, 220 aa, Artificial Sequence
  an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 101 - protein, 128 aa, Artificial Sequence
  an artificially synthesized sequence
QVQLVESGGGLVQPGRSLRLSCAASGFKFSNVWMHWVRQAPGKGLEWVAQIKDKWNAYAAYYAPSVKGRFTISRDDSKNSIYLQMNSLKTEDTAVYYCHYIHYASASTLLPAFGIDAWGQGTTVTVSS

SEQ ID NO: 102 - protein, 212 aa, Artificial Sequence
  an artificially synthesized sequence
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMDYKDDDDK

SEQ ID NO: 103 - protein, 189 aa, Artificial Sequence
  an artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQHHHHHHGGGGSGLNDIFEAQKIEWHE

SEQ ID NO: 104 - protein, 128 aa, Artificial Sequence
  an artificially synthesized sequence
QVQLVESGGGLVQPGRSLRLSCAASGFKFSNTWMHWVRQAPGKGLEWVAQIKDKYNAYAAYYAPSVKGRFTISRDDSKNSIYLQMNSLKTEDTAVYYCHYIHYASASTLLPAFGVDAWGQGTTVTVSS

SEQ ID NO: 105 - protein, 22 aa, Artificial Sequence
  an artificially synthesized sequence
LQDPCSNCPAGTFCDNNRNQIC

SEQ ID NO: 106 - protein, 128 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAFGVDAWGQGTTVTVSS

SEQ ID NO: 107 - protein, 128 aa, Artificial Sequence
  an artificially synthesized sequence
QVQLVESGGGLVQPGRSLRLSCAASGFKFSHVWFHWVRQAPGKGLEWVAQIKDKYNAYAAYYAPSVKGRFTISRDDSKNSIYLQMNSLKTEDTAVYYCHYVHYASASTLLPAFGVDAWGQGTTVTVSS

SEQ ID NO: 108 - protein, 220 aa, Artificial Sequence
  an artificially synthesized sequence
CPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 109 - protein, 220 aa, Artificial Sequence
  an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 110 - protein, 128 aa, Artificial Sequence
  an artificially synthesized sequence
QVQLVESGGGLVQPGRSLRLSCAASGFKFSNVWFHWVRQAPGKGLEWVAQIKDKYNAYAAYYAPSVKGRFTISRDDSKNSIYLQMNSLKTEDTAVYYCHYVHYASASTLLPAEGVDAWGQGTTVTVSS

SEQ ID NO: 111 - protein, 220 aa, Artificial Sequence
  an artificially synthesized sequence
CPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 112 - protein, 220 aa, Artificial Sequence
  an artificially synthesized sequence
CPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 113 - protein, 128 aa, Artificial Sequence
  an artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCRYVHYASASTLLPAFGVDAWGQGTTVTVSS

SEQ ID NO: 114 - protein, 5 aa, Artificial Sequence
  an artificially synthesized sequence
NVWMH

SEQ ID NO: 115 - protein, 11 aa, Artificial Sequence
  an artificially synthesized sequence
QASQSISTALA

SEQ ID NO: 116 - protein, 11 aa, Artificial Sequence
  an artificially synthesized sequence
QASQSISTALA

SEQ ID NO: 117 - protein, 5 aa, Artificial Sequence
  an artificially synthesized sequence
NTWMH

SEQ ID NO: 118 - protein, 11 aa, Artificial Sequence
  an artificially synthesized sequence
QASQSISTALA

SEQ ID NO: 119 - protein, 5 aa, Artificial Sequence
  an artificially synthesized sequence
NVWFH

SEQ ID NO: 120 - protein, 5 aa, Artificial Sequence
  an artificially synthesized sequence
HVWFH

SEQ ID NO: 121 - protein, 7 aa, Artificial Sequence
  an artificially synthesized sequence
GASTLES

SEQ ID NO: 122 - protein, 7 aa, Artificial Sequence
  an artificially synthesized sequence
GASTLES

SEQ ID NO: 123 - protein, 5 aa, Artificial Sequence
  an artificially synthesized sequence
NVWFH

SEQ ID NO: 124 - protein, 7 aa, Artificial Sequence
  an artificially synthesized sequence
GASTLES

SEQ ID NO: 125 - protein, 7 aa, Artificial Sequence
  an artificially synthesized sequence
GASTLES

SEQ ID NO: 126 - protein, 5 aa, Artificial Sequence
  an artificially synthesized sequence
NAWMH

SEQ ID NO: 127 - protein, 19 aa, Artificial Sequence
  an artificially synthesized sequence
QIKDKWNAYAAYYAPSVKG

SEQ ID NO: 128 - protein, 7 aa, Artificial Sequence
  an artificially synthesized sequence
GASILES

SEQ ID NO: 129 - protein, 12 aa, Artificial Sequence
  an artificially synthesized sequence
QCTYYGSSYGNA

SEQ ID NO: 130 - protein, 19 aa, Artificial Sequence
  an artificially synthesized sequence
QIKDKYNAYAAYYAPSVKG

SEQ ID NO: 131 - protein, 12 aa, Artificial Sequence
  an artificially synthesized sequence
QCTYYGNNYGNA

SEQ ID NO: 132 - protein, 19 aa, Artificial Sequence
  an artificially synthesized sequence
QIKDYYNAYAAYYAPSVKG

SEQ ID NO: 133 - protein, 19 aa, Artificial Sequence
  an artificially synthesized sequence
QIKDKYNAYAAYYAPSVKG

SEQ ID NO: 134 - protein, 12 aa, Artificial Sequence
  an artificially synthesized sequence
QSTYYGSSYGNA

SEQ ID NO: 135 - protein, 12 aa, Artificial Sequence
  an artificially synthesized sequence
QSTYYGNNYGNA

SEQ ID NO: 136 - protein, 19 aa, Artificial Sequence
  an artificially synthesized sequence
QIKDKYNAYAAYYAPSVKG

SEQ ID NO: 137 - protein, 12 aa, Artificial Sequence
  an artificially synthesized sequence
QSTYYGISYGNA

SEQ ID NO: 138 - protein, 433 aa, Artificial Sequence
  an artificially synthesized sequence
QSLEESGGRLVTPGTPLTLTCTASGFSLSTYYMNWVRQAPGKGLEWIGTITIDGTAYYASWAKGRFTISRTSTTVDLKAASPTTEDTATYFCARYDGDIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 139 - protein, 19 aa, Artificial Sequence
  an artificially synthesized sequence
QIKDKGNAYAAYYAPSVKG

SEQ ID NO: 140 - protein, 17 aa, Artificial Sequence
  an artificially synthesized sequence
IHYASASTLLPAFGIDA

SEQ ID NO: 141 - protein, 214 aa, Artificial Sequence
  an artificially synthesized sequence
DVVMTQTPASVSEPVGGTVTIKCQASQSISTALAWYQQKPGQPPKLLIHGASILESGVPSRFKGSGSGTEFTLTISDLECADVATYYCQSTYYGISYGNAFGGGTKVEIKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 142 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 143 - protein, 17 aa, Artificial Sequence
  an artificially synthesized sequence
IHYASASTLLPAFGVDA

SEQ ID NO: 144 - skipped

SEQ ID NO: 145 - protein, 17 aa, Artificial Sequence
  an artificially synthesized sequence
VHYASASTLLPAFGVDA

SEQ ID NO: 146 - protein, 17 aa, Artificial Sequence
  an artificially synthesized sequence
VHYASASTLLPAFGVDA

SEQ ID NO: 147 - protein, 226 aa, Artificial Sequence
  an artificially synthesized sequence
QVTLRESGPALVKPTQTLTLTCTFSGFSLSSSYDMGWVRQAPGQGLEWMGTIYTGDYSTDYASWAKGRVTISVDRSKNQFSLKLSSVTAADTAVYYCARHTGYGYFGLWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 149 - protein, 17 aa, Artificial Sequence
  an artificially synthesized sequence
VHYASASTLLPAEGVDA

SEQ ID NO: 150-151 - skipped (2 entries)

SEQ ID NO: 152 - protein, 17 aa, Artificial Sequence
  an artificially synthesized sequence
VHYASASTLLPAFGVDA

SEQ ID NO: 153 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 154 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 155 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 156 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 157 - protein, 219 aa, Artificial Sequence
  an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 458 aa, Artificial Sequence
  an artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELRRGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSPDYKDDDDK

SEQ ID NO: 159 - protein, 219 aa, Artificial Sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 447 aa, Artificial Sequence
  an artificially synthesized sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 161 - protein, 214 aa, Artificial Sequence [sequence omitted]

SEQ ID NO: 162 - protein, 110 aa, Artificial Sequence
  an artificially synthesized sequence
QSLEESGGRLVTPGTPLTLTCTASGFSLSSYWMNWVRQAPGKGLEWIGTVNIDGSTDYASWAKGRFTISRTSTTVDLKAASPTTEDTATYFCARYGGDIWGPGTLVIVSL

SEQ ID NO: 163 - protein, 110 aa, Artificial Sequence
  an artificially synthesized sequence
QSLEESGGRLVTPGTPLTLTCTASGFSLSTYWMNWVRQAPGKGLEWIGTINIDGSAAYASWAKGRFTISRTSTTVDLKAASPTTEDTATYFCATYGGDIWGPGTLVTVSS

SEQ ID NO: 164 - protein, 110 aa, Artificial Sequence
  an artificially synthesized sequence
QSLEESGGRLVTPGTPLTLTCTASGFSLSTYYMNWVRQAPGKGLEWIGTITIDGTAYYASWAKGRFTISRTSTTVDLKAASPTTEDTATYFCARYDGDIWGPGTLVTVSS

SEQ ID NO: 165 - protein, 112 aa, Artificial Sequence
  an artificially synthesized sequence
QSLEESGGRLVTPGTPLTLTCTASGFSLSSYYMSWVRQAPGEGLEYIGWISSDGSTYYASWAKGRFTISRTSTTVDLKITSPTTEDTATYFCARDYAASNIWGPGTLVTVSS

SEQ ID NO: 166 - protein, 5 aa, Artificial Sequence
  an artificially synthesized sequence
SYWMN

SEQ ID NO: 167 - protein, 5 aa, Artificial Sequence
  an artificially synthesized sequence
TYWMN

SEQ ID NO: 168 - protein, 5 aa, Artificial Sequence
  an artificially synthesized sequence
TYYMN

SEQ ID NO: 169 - protein, 5 aa, Artificial Sequence
  an artificially synthesized sequence
SYYMS

SEQ ID NO: 170 - protein, 16 aa, Artificial Sequence
  an artificially synthesized sequence
TVNIDGSTDYASWAKG

SEQ ID NO: 171 - protein, 16 aa, Artificial Sequence
  an artificially synthesized sequence
TINIDGSAAYASWAKG

SEQ ID NO: 172 - protein, 16 aa, Artificial Sequence
  an artificially synthesized sequence
TITIDGTAYYASWAKG

SEQ ID NO: 173 - protein, 16 aa, Artificial Sequence
  an artificially synthesized sequence
WISSDGSTYYASWAKG

SEQ ID NO: 174 - protein, 5 aa, Artificial Sequence
  an artificially synthesized sequence
YGGDI

SEQ ID NO: 175 - protein, 5 aa, Artificial Sequence
  an artificially synthesized sequence
YGGDI

SEQ ID NO: 176 - protein, 5 aa, Artificial Sequence
  an artificially synthesized sequence
YDGDI

SEQ ID NO: 177 - protein, 7 aa, Artificial Sequence
  an artificially synthesized sequence
DYAASNI

SEQ ID NO: 178 - protein, 110 aa, Artificial Sequence
  an artificially synthesized sequence
DVVMTQTPASVSEPVGGTVTIKCQASQSISTALAWYQQKPGQPPKLLIYGASTLESGVPSRFKGSGSGTEFTLTISDLECADAATYYCQCTYYGSSYGNAFGGGTKVEIK

SEQ ID NO: 179 - protein, 110 aa, Artificial Sequence
  an artificially synthesized sequence
DVVMTQTPASVSEPVGGTVTIKCQASQSISTALAWYQQKPGQPPKLLIYGASTLESGVPSRFKGSGSGTEFTLTISDLECADAATYYCQCTYYGNNYGNAFGGGTKVEIK

SEQ ID NO: 180 - protein, 110 aa, Artificial Sequence
  an artificially synthesized sequence
DVVMTQTPASVSEPVGGTVTIKCQASQSISTALAWYQQKPGQPPKLLIHGASILESGVPSRFKGSGSGTEFTLTISDLECADVATYYCQCTYYGISYGNAFGGGTKVEIK

SEQ ID NO: 181 - protein, 110 aa, Artificial Sequence
  an artificially synthesized sequence
DVVMTQTPASVSEPVGGTVTIKCQASENIGNALAWYQQKPGQRPKLLIYGASNLESGVPSRFKGSRSGTEYTLTISDLECDDAATYYCQSTYYSSSYSNPFGGGTKVEIK

SEQ ID NO: 182 - protein, 11 aa, Artificial Sequence
  an artificially synthesized sequence
QASQSISTALA

SEQ ID NO: 183 - protein, 11 aa, Artificial Sequence
  an artificially synthesized sequence
QASQSISTALA

SEQ ID NO: 184 - protein, 11 aa, Artificial Sequence
  an artificially synthesized sequence
QASQSISTALA

SEQ ID NO: 185 - protein, 11 aa, Artificial Sequence
  an artificially synthesized sequence
QASENIGNALA

SEQ ID NO: 186 - protein, 7 aa, Artificial Sequence
  an artificially synthesized sequence
GASTLES

SEQ ID NO: 187 - protein, 7 aa, Artificial Sequence
  an artificially synthesized sequence
GASTLES

SEQ ID NO: 188 - protein, 7 aa, Artificial Sequence
  an artificially synthesized sequence
GASILES

SEQ ID NO: 189 - protein, 7 aa, Artificial Sequence
  an artificially synthesized sequence
GASNLES

SEQ ID NO: 190 - protein, 12 aa, Artificial Sequence
  an artificially synthesized sequence
QCTYYGSSYGNA

SEQ ID NO: 191 - protein, 12 aa, Artificial Sequence
  an artificially synthesized sequence
QCTYYGNNYGNA

SEQ ID NO: 192 - protein, 12 aa, Artificial Sequence
  an artificially synthesized sequence
QCTYYGISYGNA

SEQ ID NO: 193 - protein, 12 aa, Artificial Sequence
  an artificially synthesized sequence
QSTYYSSSYSNP

SEQ ID NO: 194 - protein, 110 aa, Artificial Sequence
  an artificially synthesized sequence
DVVMTQTPASVSEPVGGTVTIKCQASQSISTALAWYQQKPGQPPKLLIYGASTLESGVPSRFKGSGSGTEFTLTISDLEAADAATYYCQCTYYGSSYGNAFGGGTKVEIK

SEQ ID NO: 195 - protein, 110 aa, Artificial Sequence
  an artificially synthesized sequence
DVVMTQTPASVSEPVGGTVTIKCQASQSISTALAWYQQKPGQPPKLLIYGASTLESGVPSRFKGSGSGTEFTLTISDLEAADAATYYCQCTYYGNNYGNAFGGGTKVEIK

SEQ ID NO: 196 - protein, 110 aa, Artificial Sequence
  an artificially synthesized sequence
DVVMTQTPASVSEPVGGTVTIKCQASQSISTALAWYQQKPGQPPKLLIYGASTLESGVPSRFKGSGSGTEFTLTISDLECADAATYYCQSTYYGSSYGNAFGGGTKVEIK

SEQ ID NO: 197 - protein, 110 aa, Artificial Sequence
  an artificially synthesized sequence
DVVMTQTPASVSEPVGGTVTIKCQASQSISTALAWYQQKPGQPPKLLIYGASTLESGVPSRFKGSGSGTEFTLTISDLECADAATYYCQSTYYGNNYGNAFGGGTKVEIK

SEQ ID NO: 198 - protein, 110 aa, Artificial Sequence
  an artificially synthesized sequence
DVVMTQTPASVSEPVGGTVTIKCQASQSISTALAWYQQKPGQPPKLLIHGASILESGVPSRFKGSGSGTEFTLTISDLECADVATYYCQSTYYGISYGNAFGGGTKVEIK

SEQ ID NO: 199 - protein, 11 aa, Artificial Sequence
  an artificially synthesized sequence
QASQSISTALA

SEQ ID NO: 200 - protein, 11 aa, Artificial Sequence
  an artificially synthesized sequence
QASQSISTALA

SEQ ID NO: 201 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 202 - protein, 678 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 203 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 204 - protein, 676 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 205 - protein, 671 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 206 - protein, 226 aa, Artificial Sequence; identical to SEQ ID NO: 147
  an artificially synthesized sequence

SEQ ID NO: 207 - protein, 223 aa, Artificial Sequence
  an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 209 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 210 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 211 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 212 - protein, 456 aa, Artificial Sequence
  an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQEGNVFSCSVLHEALHAHYTRKELSLSP

SEQ ID NO: 213 - protein, 456 aa, Artificial Sequence
  an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 214 - protein, 219 aa, Artificial Sequence; identical to SEQ ID NO: 157
  an artificially synthesized sequence

SEQ ID NO: 215 - protein, 219 aa, Artificial Sequence
  an artificially synthesized sequence
DIVMTQSPLSLPVTPGEPASISCQPSQEVVHMNRNTYLHWYQQKPGQAPRLLIYKVSNVFPGVPDRFSGSGSGTDFTLKISRVEAEDVGVYYCAQGTHHPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 681 aa, Artificial Sequence; identical to SEQ ID NO: 142
  an artificially synthesized sequence

SEQ ID NO: 217 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 218 - protein, 678 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 219 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 220 - protein, 676 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 221 - protein, 671 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 222 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 223 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 224 - protein, 678 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQEGNVFSCSVLHEALHAHYTRKELSLSP

SEQ ID NO: 225 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 226 - protein, 676 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 227 - protein, 671 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 228 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 229 - protein, 456 aa, Artificial Sequence; identical to SEQ ID NO: 148
  an artificially synthesized sequence

SEQ ID NO: 230 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 231 - protein, 456 aa, Artificial Sequence
  an artificially synthesized sequence
QVQLVESGGGLVQPGRSLRLSCAASGFVFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 232 - protein, 119 aa, Artificial Sequence
  an artificially synthesized sequence
QVTLRESGPALVKPTQTLTLTCTFSGFSLSSSYDMGWVRQAPGQGLEWMGTIYTGDYSTDYASWAKGRVTISVDRSKNQFSLKLSSVTAADTAVYYCARHTGYGYFGLWGQGTLVTVSS

SEQ ID NO: 233 - protein, 6 aa, Artificial Sequence
  an artificially synthesized sequence
SSYDMG

SEQ ID NO: 234 - protein, 17 aa, Artificial Sequence
  an artificially synthesized sequence
TIYTGDYSTDYASWAKG

SEQ ID NO: 235 - protein, 9 aa, Artificial Sequence
  an artificially synthesized sequence
HTGYGYFGL

SEQ ID NO: 236 - protein, 110 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIK

SEQ ID NO: 237 - protein, 13 aa, Artificial Sequence
  an artificially synthesized sequence
QSTESVYGSDWLS

SEQ ID NO: 238 - protein, 7 aa, Artificial Sequence
  an artificially synthesized sequence
QASNLEI

SEQ ID NO: 239 - protein, 10 aa, Artificial Sequence
  an artificially synthesized sequence
QGYYSGYIYA

SEQ ID NO: 240 - protein, 116 aa, Artificial Sequence
  an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 241 - protein, 5 aa, Artificial Sequence
  an artificially synthesized sequence
SYWMH

SEQ ID NO: 242 - protein, 17 aa, Artificial Sequence
  an artificially synthesized sequence
MIDPSYSETRLNQKFKD

SEQ ID NO: 243 - protein, 7 aa, Artificial Sequence
  an artificially synthesized sequence
YGNYFDY

SEQ ID NO: 244 - protein, 107 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 245 - protein, 11 aa, Artificial Sequence
  an artificially synthesized sequence
KASEDIYNRLA

SEQ ID NO: 246 - protein, 7 aa, Artificial Sequence
  an artificially synthesized sequence
GATSLET

SEQ ID NO: 247 - protein, 9 aa, Artificial Sequence
  an artificially synthesized sequence
QQYWSTPYT

SEQ ID NO: 248 - protein, 11 aa, Artificial Sequence
  an artificially synthesized sequence
VEPKSCGGGGS

SEQ ID NO: 249 - protein, 16 aa, Artificial Sequence
  an artificially synthesized sequence
VEPKSCGGGGSGGGGS

SEQ ID NO: 250 - protein, 984 aa, Artificial Sequence
  an artificially synthesized sequence
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 122 aa, Artificial Sequence; identical to SEQ ID NO: 77
  an artificially synthesized sequence

SEQ ID NO: 252 - protein, 5 aa, Artificial Sequence
  an artificially synthesized sequence
NAWMH

SEQ ID NO: 253 - protein, 19 aa, Artificial Sequence
  an artificially synthesized sequence
QIKDKSQNYATYVAESVKG

SEQ ID NO: 254 - protein, 11 aa, Artificial Sequence
  an artificially synthesized sequence
VHYAAGYGVDI

SEQ ID NO: 255 - protein, 112 aa, Artificial Sequence; identical to SEQ ID NO: 78
  an artificially synthesized sequence

SEQ ID NO: 256 - protein, 16 aa, Artificial Sequence
  an artificially synthesized sequence
RSSQPLVHSNRNTYLH

SEQ ID NO: 257 - protein, 7 aa, Artificial Sequence
  an artificially synthesized sequence
KVSNRFS

SEQ ID NO: 258 - protein, 9 aa, Artificial Sequence
  an artificially synthesized sequence
GQGTQVPYT

SEQ ID NO: 259 - protein, 6 aa, Artificial Sequence
  an artificially synthesized sequence
VEPKSC

SEQ ID NO: 260 - protein, 122 aa, Artificial Sequence
  an artificially synthesized sequence
QSVEESGGRLVTPGTPLTLTCTVSGIDLSSYAMSWVRQAPGKGLEWIGIISNSGSGFYANWAKGRFTISKTSSTTVDVKISSPTTEDTATYFCARVFDADSSGYYYYGMDPWGPGTLVTVSS

SEQ ID NO: 261 - protein, 111 aa, Artificial Sequence
  an artificially synthesized sequence
DVVMTQTPSSVSEPVGGTVTIKCQASEDIESYLAWYQQKPGQPPKLLIYAASTLASGVSSRFRGSGSGTEYTLTISDLECADAATYYCQSTYYDSSGVDWAFGGGTEVVVK

SEQ ID NO: 262 - protein, 123 aa, Artificial Sequence
  an artificially synthesized sequence
QSVEESGGRLVTPGTPLTLTCTVSGFSLSRYAMSWVRQAPGKGLEWIGIITGGGDPYYASWAKGRFTISKTSTTMDLKITSPTTEDTATYFCARMIGSEYASSSEYYDAFDPWGPGTLVTVSS

SEQ ID NO: 263 - protein, 112 aa, Artificial Sequence
  an artificially synthesized sequence
ALVMTQTPSSVSAAVGGTVTINCQASQNIYSNLAWYQQKPGQRPKLLIYGASNLESGVPSRFKGSGSGTEYTLTISDLECDDAAIYYCQSAYYTTSVDVLYPFGGGTEVVVK

SEQ ID NO: 264 - protein, 124 aa, Artificial Sequence
  an artificially synthesized sequence
QEQLVESGGGLVQPGGSLKLSCKVSGFDFSNYGVSWVRQAPGKGLEWIGYIDPAFGRTYYASWVNGRFTISSHNAQNTLYLQLNSLTAADTATYFCVRVALVVAGVAYYGMDVWGPGTLVTVSS

SEQ ID NO: 265 - protein, 114 aa, Artificial Sequence
  an artificially synthesized sequence
ADVVMTQTPASVEAAVGGTVTINCQASQNIYSYLSWYQQKPGQPPKLLIYRTSTLASGVPSRFKGSGSGTQFTLTISDLECADAATYYCQSYYYSSGSSYGAFAFGGGTEVVVK

SEQ ID NO: 266 - protein, 114 aa, Artificial Sequence
  an artificially synthesized sequence
QSLEESGGRLVTPGTPLTLTCTASGFSLNTYHMIWVRQAPGKGLEWIGIVDSGGSTYYASWAKGRFTISRTSTTVDLKATSLTTEDTATYFCARGGSWDGFNIWGPGTLVTVSS

SEQ ID NO: 267 - protein, 112 aa, Artificial Sequence
  an artificially synthesized sequence
DVVMTQTPSSVSAAVGGTVTINCQSSQSVYNSDFLAWYQQKPGQPPKLLIYAASTLASGVPSRFKGSGSGTQFTLTISDLECDDAATYYCQGTYEDVGWFNDFGGGTEVVVK

SEQ ID NO: 268 - protein, 128 aa, Artificial Sequence
  an artificially synthesized sequence
QEQLVESGGGLVQPGASLTLTCKASGFDFTSNAMCWVRQAPGKGPEWIACIYNDDGSAYSASWAKGRFTVSKTSSTAVTLQVTGLTAADTATYFCARSPYYTYGGAPSAYASGYFNLWGPGTLVTVSS

SEQ ID NO: 269 - protein, 113 aa, Artificial Sequence
  an artificially synthesized sequence
ADIVMTQTPASVEAAVGGTVTIKCQASENIYNSLAWYQQKPGQPPKLLMHDASVLTSGVPSRFSGSGYGTEFTLTISDLECADAATYYCQSDYYSTGGSYGFAFGGGTEVVVK

SEQ ID NO: 270 - protein, 121 aa, Artificial Sequence
  an artificially synthesized sequence
QEQLVESGGGLVQPGASLTLTCKASGFSFNTSYCPCWVRQVPGKGPEWIACIDAGYSGATWYANWAKGRFTISKTSSTTVTLQMTSLTGADTATYFCVRCDAAGSGAFNLWGPGTLVTVSS

SEQ ID NO: 271 - protein, 110 aa, Artificial Sequence
  an artificially synthesized sequence
AYDMTQTPASVEVPVGGTVTIKCQASQSIYSYLSWYQQKPGQPPKLLIYEASKLTSGVPSRFSGSGSGTEFTLTISGVQCDDAATYYCQQGSGYMDVDNVFGGGTEVVVK

SEQ ID NO: 272 - protein, 123 aa, Artificial Sequence
  an artificially synthesized sequence
QEQLVESGGGLVQPEGSLTLTCTASGFSFSSSGYMCWVRQAPGKGLEWIACISGGSSGDTDYANWAKGRFSISKTSSTTVTLQMTSLTVADTATYFCARDVYIDSTIFNFNLWGPGTLVTVSS

SEQ ID NO: 273 - protein, 111 aa, Artificial Sequence
  an artificially synthesized sequence
QVLTQTPSPVSAAVGGTVTISCQASQSVYNNNNLAWYQQKPGQPPKLLIYAASSLASGVPSRFKGSGAGTQFTLTISDLECDDAATYYCQGTYYSSGWYSTFGGGTEVVVK

SEQ ID NO: 274 - protein, 118 aa, Artificial Sequence
  an artificially synthesized sequence
QEQLVESGGGLVQPEGSLTLTCTASGFSFSSSYDMCWVRQAPGKGPEWIACIYTGDGSTDYASWAKGRFTVSKASSTTVTLQMTSLAVADTATYFCARNSGYGYFGLWGPGTLVTVSS

SEQ ID NO: 275 - protein, 109 aa, Artificial Sequence
  an artificially synthesized sequence
QVLTQTPSPVSAAVGGTVTISCQSTESVYGSDWLSWYQQKPGQPPKVLIYQASNLEIGVPSRFKGSGSGTQFTLTISGAQCDDAATYYCQGYYSGYIYAFGGGTEVVVK

SEQ ID NO: 276 - protein, 5 aa, Artificial Sequence
  an artificially synthesized sequence
NYGVS

SEQ ID NO: 277 - protein, 17 aa, Artificial Sequence
  an artificially synthesized sequence
YIDPAFGRTYYASWVNG

SEQ ID NO: 278 - protein, 15 aa, Artificial Sequence
  an artificially synthesized sequence
VALVVAGVAYYGMDV

SEQ ID NO: 279 - protein, 11 aa, Artificial Sequence
  an artificially synthesized sequence
QASQNIYSYLS

SEQ ID NO: 280 - protein, 7 aa, Artificial Sequence
  an artificially synthesized sequence
RTSTLAS

SEQ ID NO: 281 - protein, 15 aa, Artificial Sequence
  an artificially synthesized sequence
QSYYYSSGSSYGAFA

SEQ ID NO: 282 - protein, 6 aa, Artificial Sequence
  an artificially synthesized sequence
SSYDMC

SEQ ID NO: 283 - protein, 17 aa, Artificial Sequence
  an artificially synthesized sequence
CIYTGDGSTDYASWAKG

SEQ ID NO: 284 - protein, 9 aa, Artificial Sequence
  an artificially synthesized sequence
NSGYGYFGL

SEQ ID NO: 285 - protein, 5 aa, Artificial Sequence
  an artificially synthesized sequence
SNAMC

SEQ ID NO: 286 - protein, 17 aa, Artificial Sequence
  an artificially synthesized sequence
CIYNDDGSAYSASWAKG

SEQ ID NO: 287 - protein, 20 aa, Artificial Sequence
  an artificially synthesized sequence
SPYYTYGGAPSAYASGYFNL

SEQ ID NO: 288 - protein, 11 aa, Artificial Sequence
  an artificially synthesized sequence
QASENIYNSLA

SEQ ID NO: 289 - protein, 7 aa, Artificial Sequence
  an artificially synthesized sequence
DASVLTS

SEQ ID NO: 290 - protein, 14 aa, Artificial Sequence
  an artificially synthesized sequence
QSDYYSTGGSYGFA

SEQ ID NO: 291 - protein, 6 aa, Artificial Sequence
  an artificially synthesized sequence
SSGYMC

SEQ ID NO: 292 - protein, 18 aa, Artificial Sequence
  an artificially synthesized sequence
CISGGSSGDTDYANWAKG

SEQ ID NO: 293 - protein, 13 aa, Artificial Sequence
  an artificially synthesized sequence
DVYIDSTIFNFNL

SEQ ID NO: 294 - protein, 13 aa, Artificial Sequence
  an artificially synthesized sequence
QASQSVYNNNNLA

SEQ ID NO: 295 - protein, 7 aa, Artificial Sequence
  an artificially synthesized sequence
AASSLAS

SEQ ID NO: 296 - protein, 12 aa, Artificial Sequence
  an artificially synthesized sequence
QGTYYSSGWYST

SEQ ID NO: 297 - protein, 119 aa, Artificial Sequence
  an artificially synthesized sequence
QVQLKESGPALVKPTQTLTLTCTFSGFSLSSSYDMGWVRQAPGQGLEWMGTIYTGDYSTDYASWAKGRVTITADESTSTAYMELSSLRSEDTAVYYCARNTGYGYFGLWGQGTLVTVSS

SEQ ID NO: 298 - protein, 119 aa, Artificial Sequence
  an artificially synthesized sequence
QVTLRESGPALVKPTQTLTLTCTFSGFSLSSSYDMGWVRQAPGQGLEWMGTIYTGDYSTDYASWAKGRVTISVDTSKNQFSLKLSSVTAADTAVYYCARHTGYGYFGLWGQGTLVTVSS

SEQ ID NO: 299 - protein, 119 aa, Artificial Sequence
  an artificially synthesized sequence
QVTLRESGPALVKPTQTLTLTCAFSGFSLSSSYDMGWVRQAPGQGLEWMGTIYTGDYSTDYASWAKGRVTISVDTSKNQFSLKLSSVTAADTAVYYCARHTGYGYFGLWGQGTLVTVSS

SEQ ID NO: 300 - protein, 119 aa, Artificial Sequence
  an artificially synthesized sequence
QVTLRESGPALVKPTQTLTLTCTFSGFSLSSSYDMGWVRQAPGQGLEWMGTIYTGDYSTDYASWAKGRVTISVDSSKNQFSLKLSSVTAADTAVYYCARHTGYGYFGLWGQGTLVTVSS

SEQ ID NO: 301 - protein, 119 aa, Artificial Sequence
  an artificially synthesized sequence
QVTLRESGPALVKPTQTLTLTCTFSGFSLSSSYDMGWVRQAPGQGLEWMGTIYTGDYSTDYASWAKGRVTISVDKSKNQFSLKLSSVTAADTAVYYCARHTGYGYFGLWGQGTLVTVSS

SEQ ID NO: 302 - protein, 110 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYSYAFGGGTKVEIK

SEQ ID NO: 303 - protein, 9 aa, Artificial Sequence
  an artificially synthesized sequence
NTGYGYFGL

SEQ ID NO: 304 - protein, 10 aa, Artificial Sequence
  an artificially synthesized sequence
QGYYSGYSYA

SEQ ID NO: 305 - protein, 121 aa, Artificial Sequence
  an artificially synthesized sequence
QVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSS

SEQ ID NO: 306 - protein, 118 aa, Artificial Sequence
  an artificially synthesized sequence
EVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSS

SEQ ID NO: 307 - protein, 119 aa, Artificial Sequence
  an artificially synthesized sequence
QVQLQQSGGDLMKPGASVKISCKAAGYTFSNYYIEWVKQRPGHGLEWIGEILPGSGSTTYNEKFKGKASFTADTSSNTAYMQLSSLTSEDSAVYYCARWGAREPGFPYWGQGTLVTVSA

SEQ ID NO: 308 - protein, 123 aa, Artificial Sequence
  an artificially synthesized sequence
EVKLVESGGGLVQPGGSLSLSCAASGFTFTDYYMNWVRQPPGKALEWLALIRNKANGYTTEYNASVKGRFTISRDNSQNILYLQMNALRAEDSATYYCARDSDGYYEYYFDYWGQGTTLTVSS

SEQ ID NO: 309 - protein, 123 aa, Artificial Sequence
  an artificially synthesized sequence
QVQLVQSGAEVKKPGASVKVSCKASGYTFTNYYMHWVRQAPGLGLEWMGIINPSDGSTSYAQKFQGRVTMTRDTSTNTVYMDLSSLRSEDTAVYYCARGGNSAFYSYYDMDVWGQGTTVTVSS

SEQ ID NO: 310 - protein, 118 aa, Artificial Sequence
  an artificially synthesized sequence
QVQLQESGPGLVKPSETLSLTCTVSGDSISSYYWTWIRQPPGKGLEWIGYIYYSGTTNYNPSLKSRVTISVDTSKSQFSLKLSSVTAADTAVYYCASIAVRGFFFDYWGQGTLVTVSS

SEQ ID NO: 311 - protein, 118 aa, Artificial Sequence
  an artificially synthesized sequence
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 312 - protein, 118 aa, Artificial Sequence
  an artificially synthesized sequence
QVQLQESGPGLVKPSETLSLTCTVSGASISSFYWSWIRQPPGKGLEWIGYIYYSGTTNYNPSLKSRVTISVDTSKNQFSLKLSSVTAADTAVYYCARIAVAGFFFDYWGQGTLVTVSS

SEQ ID NO: 313 - protein, 107 aa, Artificial Sequence
  an artificially synthesized sequence
DIKMTQSPSSMYASLGERVTITCKASQDINSYLIWFQQKPGKSPKTLIYRTNRLVDGVPSRFSGSGSGQDYSLTISSLEYGDMGIYYCLQYDEFPFTFGSGTKLEIK

SEQ ID NO: 314 - protein, 111 aa, Artificial Sequence
  an artificially synthesized sequence
DIVLTQSPASLAVSLGQRATISCRASKSVSTSGYSYMHWYQQKPGQPPKLLIFLASNLESGVPARFSGSGSGTDFTLNIHPVEEEDAATYYCQHSRHLPWTFGGGTKLEIK

SEQ ID NO: 315 - protein, 107 aa, Artificial Sequence
  an artificially synthesized sequence
DIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELK

SEQ ID NO: 316 - protein, 107 aa, Artificial Sequence
  an artificially synthesized sequence
DIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIK

SEQ ID NO: 317 - protein, 112 aa, Artificial Sequence
  an artificially synthesized sequence
DVVMTQTPLSLPVTLGQPASISCRSSQSLVYRDGNTYLSWFQQRPGQSPRRLIYKVSNWQSGVPDRFSGGGSGTDFTLKISRVEAEDVGVYYCMQGTHWPPTFGQGTKVEIK

SEQ ID NO: 318 - protein, 108 aa, Artificial Sequence
  an artificially synthesized sequence
EIVLTQSPGTLSLSPGERATLSCRASQSVSSSYLAWYQQKPGQAPRLLIYGASTRATGIPDRFSGSGSGTDFTLTISRLEPEDFAVYYCQQYGTSPLTFGGGTKVEIK

SEQ ID NO: 319 - protein, 108 aa, Artificial Sequence
  an artificially synthesized sequence
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 320 - protein, 108 aa, Artificial Sequence
  an artificially synthesized sequence
EIVLTQSPGTLSLSPGERATLSCRASQSVNKNYLAWYQQKPGQAPRLLIYGASSRATGIPDRFSGSGSGTDFTLTISRLEPEDFAVYYCQQYDRSPLTFGGGTKVEIK

SEQ ID NO: 321 - protein, 683 aa, Artificial Sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 322 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 323 - protein, 678 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 324 - protein, 683 aa, Artificial Sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 325 - protein, 681 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 326 - protein, 676 aa, Artificial Sequence
  an artificially synthesized sequence
DIQLTQSPSFLSASVGDRVTITCQSTESVYGSDWLSWYQQKPGQPPKLLIYQASNLEIGVPSRFSGSGSGTDFTLTINSLEAEDAATYYCQGYYSGYIYAFGGGTKVEIKSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 327 - protein, 222 aa, Artificial Sequence
  an artificially synthesized sequence
QVQLVQSGAEVKKPGASVTVSCKASGYTFTDYEMHWIRQPPGEGLEWIGAIDGPTPDTAYSEKFKGRVTLTADKSTSTAYMELSSLTSEDTAVYYCTRFYSYTYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSCSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 329 - protein, 456 aa, Artificial Sequence; identical to SEQ ID NO: 156
  an artificially synthesized sequence

SEQ ID NO: 330 - protein, 456 aa, Artificial Sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 331 - protein, 115 aa, Artificial Sequence
  an artificially synthesized sequence
QVQLVQSGAEVKKPGASVTVSCKASGYTFTDYEMHWIRQPPGEGLEWIGAIDGPTPDTAYSEKFKGRVTLTADKSTSTAYMELSSLTSEDTAVYYCTRFYSYTYWGQGTLVTVSS

SEQ ID NO: 332 - protein, 5 aa, Artificial Sequence
  an artificially synthesized sequence
DYEMH

SEQ ID NO: 333 - protein, 17 aa, Artificial Sequence
  an artificially synthesized sequence
AIDGPTPDTAYSEKFKG

SEQ ID NO: 334 - protein, 6 aa, Artificial Sequence
  an artificially synthesized sequence
FYSYTY

SEQ ID NO: 335 - protein, 112 aa, Artificial Sequence; identical to SEQ ID NO: 78
  an artificially synthesized sequence

SEQ ID NO: 336 - protein, 16 aa, Artificial Sequence
  an artificially synthesized sequence
RSSQPLVHSNRNTYLH

SEQ ID NO: 337 - protein, 7 aa, Artificial Sequence
  an artificially synthesized sequence
KVSNRFS

SEQ ID NO: 338 - protein, 9 aa, Artificial Sequence
  an artificially synthesized sequence
GQGTQVPYT

SEQ ID NO: 339 - protein, 683 aa, Artificial Sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELRRGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

(19) United States
(12) Patent Application Publication
(10) Pub. No.: US 2023/0121511 A1
(43) Pub. Date: Apr. 20, 2023

(54) METHOD FOR PRODUCING MULTISPECIFIC ANTIGEN-BINDING MOLECULES

(71) Applicant: Chugai Seiyaku Kabushiki Kaisha, Tokyo (JP)

(72) Inventors: Vishnu Priyanka Reddy Chichili, Singapore (SG); Chai Ling Pang, Singapore (SG); Tatsuya Kawa, Shizuoka (JP); Shu Feng, Singapore (SG); Siok Wan Gan, Singapore (SG); Noriyuki Takahashi, Singapore (SG); Masaru Muraoka, Shizuoka (JP)

(73) Assignee: Chugai Seiyaku Kabushiki Kaisha, Tokyo (JP)

(21) Appl. No.: 17/914,432

(22) PCT Filed: Mar. 31, 2021
(86) PCT No.: PCT/JP2021/013795

§ 371 Date: Sep. 26, 2022

(30) Foreign Application Priority Data
Mar. 31, 2020 (JP) 2020-062601

(51) Int. Cl. C07K 16/28; C07K 16/18

(52) U.S. Cl. CPC C07K 16/2878; C07K 16/2809; C07K 16/18; C07K 2317/31; C07K 2317/522; A61K 2039/545

(57) ABSTRACT

Multispecific antigen-binding molecule capable of binding to multiple different antigens, but do not non-specifically crosslink two or more immune cells such as T cells are provided. Methods for producing or enriching a preferred structural form of such multispecific antibody protein, and method for eliminating disulfide heterogeneity of such multispecific antibody proteins are provided. In addition, conformation-specific antibodies that specifically recognize the preferred form of multispecific antibody proteins, and use of the conformation-specific antibodies are provided.

### TECHNICAL FIELD

The present invention relates to multispecific antigen-binding molecules comprising two or more antigen-binding moieties which are capable of being linked with each other via at least one disulfide bond, and methods for producing such multispecific antigen-binding molecules. More particularly, the invention relates to methods for increasing or enriching a preferred form of multispecific antibody proteins, and methods for eliminating disulfide heterogeneity of such recombinant antibody proteins.

### BACKGROUND ART

Antibodies are drawing attention as pharmaceuticals since they are highly stable in plasma and have few side effects. Among multiple therapeutic antibodies, some types of antibodies require effector cells to exert an anti-tumor response. Antibody dependent cell-mediated cytotoxicity (ADCC) is a cytotoxicity exhibited by effector cells against antibody-bound cells via binding of the Fc region of the antibody to Fc receptors present on NK cells and macrophages. To date, multiple therapeutic antibodies that can induce ADCC to exert anti-tumor efficacy have been developed as pharmaceuticals for treating cancer (Nat. Biotechnol. (2005) 23, 1073-1078).

In addition to the antibodies that induce ADCC by recruiting NK cells or macrophages as effector cells, T cell-recruiting antibodies (TR antibodies) that adopt cytotoxicity by recruiting T cells as effector cells have been known since the 1980s (NPL 2 to NPL 4). A TR antibody is a bispecific antibody that recognizes and binds to any one of the subunits forming a T-cell receptor complex on T-cells, in particular the CD3 epsilon chain, and an antigen on cancer cells. Several TR antibodies are currently being developed. Catumaxomab, which is a TR antibody against EpCAM, has been approved in the EU for the treatment of malignant ascites. Furthermore, a type of TR antibody called “bispecific T-cell engager (BiTE)” has been recently found to exhibit a strong anti-tumor activity (NPL 5 and NPL 6). Blinatumomab, which is a BiTE molecule against CD19, received FDA approval first in 2014. Blinatumomab has been proved to exhibit a much stronger cytotoxic activity against CD19/CD20-positive cancer cells in vitro compared with Rituximab, which induces antibody-dependent cell-mediated cytotoxicity (ADCC) and complement-dependent cytotoxicity (CDC) (NPL 7).

However, it is known that a trifunctional antibody binds to both a T-cell and a cell such as an NK cell or macrophage at the same time in a cancer antigen-independent manner, and as a result receptors expressed on the cells are cross-linked, and expression of various cytokines is induced in an antigen-independent manner. Systemic administration of a trifunctional antibody is thought to cause cytokine storm-like side effects as a result of such induction of cytokine expression. In fact, it has been reported that, in the phase I clinical trial, a very low dose of 5 microgram/body was the maximum tolerance dose for systemic administration of catumaxomab to patients with non-small cell lung cancer, and that administration of a higher dose causes various severe side effects (NPL 8). When administered at such a low dose, catumaxomab can never reach the effective blood level. That is, the expected anti-tumor effect cannot be achieved by administrating catumaxomab at such a low dose.

In recent years, a modified antibody that causes cytotoxic activity mediated by T cells while circumventing adverse reactions has been provided by use of an Fc region having reduced binding activity against Fc gamma R (PTL 1). Even such an antibody, however, fails to act on two immunoreceptors, i.e., CD3 epsilon and Fc gamma R, while binding to the cancer antigen, in view of its molecular structure. An antibody that exerts both of cytotoxic activity mediated by T cells and cytotoxic activity mediated by cells other than the T cells in a cancer antigen-specific manner while circumventing adverse reactions has not yet been known.

Meanwhile, unlike catumaxomab, a bispecific sc(Fv)2 format molecule (BiTE) which has no Fc gamma receptor-binding site, and therefore it does not cross-link the receptors expressed on T-cells and cells such as NK cells and macrophages in a cancer antigen-independent manner. However, since bispecific sc(Fv)2 is a modified low-molecular-weight antibody molecule without an Fc region, the problem is that its blood half-life after administration to a patient is significantly shorter than IgG-type antibodies conventionally used as therapeutic antibodies. In fact, the blood half-life of bispecific sc(Fv)2 administered in vivo has been reported to be about several hours (NPL 9 and NPL 10). Blinatumomab, a sc(Fv)2 molecule that binds to CD19 and CD3, has been approved for treatment of acute lymphoblastic leukemia. The serum half-life of blinatumomab has been revealed to be less than 2 hours in patients (NPL 11). In the clinical trials of blinatumomab, it was administered by continuous intravenous infusion using a minipump. This administration method is not only extremely inconvenient for patients but also has the potential risk of medical accidents due to device malfunction or the like. Thus, it cannot be said that such an administration method is desirable.

T cells play important roles in tumor immunity, and are known to be activated by two signals: 1) binding of a T cell receptor (TCR) to an antigenic peptide presented by major histocompatibility complex (MHC) class I molecules and activation of TCR; and 2) binding of a costimulator on the surface of T cells to the ligands on antigen-presenting cells and activation of the costimulator. Furthermore, activation of molecules belonging to the tumor necrosis factor (TNF) superfamily and the TNF receptor superfamily, such as CD137(4-1BB) on the surface of T cells, has been described as important for T cell activation (NPL 12). In this regard, CD137 agonist antibodies have already been demonstrated to show anti-tumor effects, and this has been shown experimentally to be mainly due to activation of CD8-positive T cells and NK cells (NPL 13). It is also understood that T cells engineered to have chimeric antigen receptor molecules (CAR-T cells) which consist of a tumor antigen-binding domain as an extracellular domain and the CD3 and CD137 signal transducing domains as intracellular domains can enhance the persistence of the efficacy (Porter, N ENGL J MED, 2011, 365; 725-733 (NPL 14)). However, side effects of such CD137 agonist antibodies due to their non-specific hepatotoxicity have been a problem clinically and non-clinically, and development of pharmaceutical agents has not advanced (Dubrot, Cancer Immunol. Immunother., 2010, 28, 512-22 (NPL 15)). The main cause of the side effects has been suggested to involve binding of the antibody to the Fc gamma receptor via the antibody constant region (Schabowsky, Vaccine, 2009, 28, 512-22 (NPL 16)). Furthermore, it has been reported that for agonist antibodies targeting receptors that belong to the TNF receptor superfamily to exert an agonist activity in vivo, antibody crosslinking by Fc gamma receptor-expressing cells (Fc gamma RII-expressing cells) is necessary (Li, Proc Natl Acad Sci USA. 2013, 110(48), 19501-6 (NPL 17)). WO2015/156268 (PTL 2) describes that a bispecific antibody which has a binding domain with CD137 agonistic activity and a binding domain to a tumor specific antigen can exert CD137 agonistic activity and activate immune cells only in the presence of cells expressing the tumor specific antigen.

Tri-specific antibodies comprising a tumor-specific antigen (EGFR)-binding domain, a CD137-binding domain, and a CD3-binding domain were already reported (WO2014116846). However, since antibodies with such a molecular format can bind to three different antigens at the same time, it was speculated that those tri-specific antibodies could result in cross-linking between CD3 epsilon-expressing T cells and CD137-expressing cells (e.g. T cells, B cells, NK cells, DCs etc.) by binding to CD3 and CD137 at the same time. In this context, an antibody that exerts both cytotoxic activity mediated by T cells and activation activity of T cells and other immune cells via CD137 in a cancer antigen-specific manner while circumventing adverse reactions has not yet been known.

For antibodies having multiple disulfide bonds, structural heterogeneity among the antibody preparation has been observed, although the reasons underlying this heterogeneity have remained unexplained. For example, U.S. patent application Pub. No: 2005/0161399, Dillon et al. discusses a reversed-phase LC/MS method of analyzing high molecular weight proteins, including antibodies. In addition, U.S. patent application Pub. No: 2006/194280, Dillon et al. is directed to methods of transiently enriching particular IgG isoforms by subjecting preparations of recombinant IgG proteins with a reduction/oxidation coupling reagent and optionally a chaotropic agent.

### CITATION LIST

### Patent Literature

[PTL 1] WO2012/073985
[PTL 2] WO2015/156268
[PTL 3] WO2014116846

### Non Patent Literature

[NPL 1] Nat. Biotechnol. (2005) 23, 1073-1078
[NPL 2] Nature. 1985 Apr. 18-24; 314(6012):628-31.
[NPL 3] Int J Cancer. 1988 Apr. 15; 41(4):609-15.
[NPL 4] Proc Natl Acad Sci USA. 1986 March; 83(5):1453-7.
[NPL 5] Proc Natl Acad Sci USA. 1995 Jul. 18; 92(15):7021-5.
[NPL 6] Drug Discov Today. 2005 Sep. 15; 10(18):1237-44.
[NPL 7] Int J Cancer. 2002 Aug. 20; 100(6):690-7.
[NPL 8] Cancer Immunol Immunother (2007) 56 (10), 1637-44
[NPL 9] Cancer Immunol Immunother. (2006) 55 (5), 503-14
[NPL 10] Cancer Immunol Immunother. (2009) 58 (1), 95-109
[NPL 11] Nat Rev Drug Discov. 2014 November; 13(11):799-801.
[NPL 12] Vinay, 2011, Cellular & Molecular Immunology, 8, 281-284
[NPL 13] Houot, 2009, Blood, 114, 3431-8
[NPL 14] Porter, N ENGL J MED, 2011, 365; 725-733
[NPL 15] Dubrot, Cancer Immunol. Immunother., 2010, 28, 512-22
[NPL 16] Schabowsky, Vaccine, 2009, 28, 512-22
[NPL 17] Li, Proc Natl Acad Sci USA. 2013, 110(48), 19501-6

### SUMMARY OF INVENTION

### Technical Problem

An antibody that exerts both cytotoxic activity mediated by immune cells (e.g. T cells) and activating activity of T cells and/or other immune cells via costimulatory molecules (e.g. CD137) in a target antigen-specific manner while circumventing adverse reactions has not yet been known. An objective of the present invention is to provide antigen-binding molecules which exhibit effective target-specific cell killing efficacy mediated by immune cells (e.g. T cells) while having reduced or minimal side effects.

Another objective of the present invention is to provide methods for producing the multispecific antigen-binding molecule, methods for increasing or enriching a preferred form of multispecific antibody proteins, and methods for eliminating disulfide heterogeneity of such recombinant antibody proteins.

### Solution to Problem

Antigen-binding molecules which are capable of binding to multiple different antigens (e.g., CD3 on T cells, and CD137 on T cells, NK cells, DC cells, and/or the like), but do not non-specifically crosslink two or more immune cells such as T cells are provided. Such multispecific antigen-binding molecules are capable of modulating and/or activating an immune response while circumventing the cross-linking between different cells (e.g., different T cells) resulting from the binding of a conventional multispecific antigen-binding molecule to antigens expressed on the different cells, which is considered to be responsible for adverse reactions when the multispecific antigen-binding molecule is used as a drug.

In one aspect, the antigen-binding molecule of the present invention provides new antigen-binding molecules which have very unique structure format(s), which improve or enhance the efficacy of the multispecific antigen-binding molecules. The new antigen-binding molecules with unique structure formats provide the increased number of antigen-binding domains to give the increased valency and/or specificities to respective antigens on effector cells and target cells with the reduced unwanted adverse effects.

In a further aspect, one of the antigen-binding molecules having such new unique structure format of the present invention comprises at least two first and second antigen-binding moieties (e.g., Fab domains) which are linked together (e.g., via Fc, disulfide bond, linker, or the like), each of which binds to a first and/or second antigen on effector cells (e.g., immune cells such as T cells, NK cells, DC cells, or the like), and further comprises a third (and optionally a fourth) antigen-binding domain(s) which is linked to any one of the first or second antigen-binding moieties, which bind(s) to the third antigen on target cells (e.g., tumor cells).

In a further aspect, one of the antigen-binding molecules having such new unique structure format of the present invention comprises at least a first antigen-binding moiety and a second antigen-binding moiety (e.g., Fab domains) which are linked together (e.g., via Fc, disulfide bond, linker, or the like), each of which binds to a first and/or second antigen on effector cells (e.g., immune cells such as T cells, NK cells, DC cells, or the like), and further comprises a third (and optionally the fourth) antigen-binding moiety(s) which is linked to any one of the first or second antigen-binding moiety, which bind(s) to the third antigen on target cells (e.g., tumor cells), wherein the first and second antigen-binding moieties (e.g. Fab domains) capable of binding to the first antigen and/or a second antigen comprise at least one amino acid mutation(s) respectively, which create a disulfide linkage between the first and second antigen-binding moieties to hold them close to each other, and, for example, promote cis-antigen binding to the same single effector cell as a result of steric hindrance or shorter distance between the two Dual-Fabs, thereby improving the safety profile of the trispecific antibody (trispecific Ab) by preventing undesirable crosslinking of two CD3/CD137-expressing immune cells mediated by the two Dual-Fabs in an DLL3-independent manner. In one specific aspect, said each of the first antigen-binding moiety and the second antigen-binding moiety is a Fab and comprises at least one cysteine residue (via mutation, substitution or insertion) in the CH1 region, said at least one cysteine residue is capable of forming at least one disulfide bond between the CH1 region of the first antigen-binding moiety and the CH1 region of the second antigen-binding moiety. In another specific aspect, said each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.

The antigen-binding molecules having such unique structure formats were surprisingly found to show superior efficacy compared to other multispecific antibody formats (e.g. BiTE) while exhibiting reduced or minimal off-target side-effects attributed by undesired cross-linking among different cells (e.g., effector cells such as T cells). In one aspect, the present invention relates to multispecific antigen-binding molecules that comprise a first antigen-binding moiety and a second antigen-binding moiety, each of which is capable of binding to CD3 and CD137, but does not bind to CD3 and CD137 at the same time (i.e. capable of binding to CD3 and CD137 but not simultaneously); and a third antigen-binding moiety that is capable of binding to DLL3, preferably human DLL3, which induce T-cell dependent cytotoxity more efficiently whilst circumventing adverse toxicity concerns or side effects that other multispecific antigen-binding molecules may have. The present invention provides multispecific antigen-binding molecules and pharmaceutical compositions that can treat various cancers, especially those associated with DLL3 such as DLL3-positive tumors, by comprising the antigen-binding molecule as an active ingredient.

In another aspect, the present invention relates to methods of producing the multispecific antigen-binding molecules of novel format comprising one or more disulfide linkage between the first and second antigen-binding moieties (e.g. at the CH1 region); methods for increasing or enriching a preferred form of multispecific antibody protein having said at least one disulfide linkage, and methods for eliminating disulfide heterogeneity of such recombinant antibody proteins by contacting the antibody preparation with a reducing agent under conditions which allows said at least one disulfide linkage (e.g. at the CH1 region) to form efficiently and properly. In a further aspect, the present invention relates to conformation-specific antibodies that specifically recognize the preferred form of multispecific antibody proteins, and use of the conformation-specific antibodies in purification, analytical or quantification of antibody-containing samples.

In one specific aspect, the present disclosure provides the following:
[1] A multispecific antigen-binding molecule comprising:
a first antigen-binding moiety and a second antigen-binding moiety, each of which is capable of binding to CD3 and CD137, but does not bind to CD3 and CD137 at the same time; and
a third antigen-binding moiety that is capable of binding to a third antigen, preferably an antigen expressed on a cancer cell/tissue.
[1A] A multispecific antigen-binding molecule comprising:
a first antigen-binding moiety and a second antigen-binding moiety, each of which is capable of binding to CD3 and CD137, but does not bind to CD3 and CD137 at the same time; and
a third antigen-binding moiety that is capable of binding to DLL3, preferably human DLL3.
[2] The multispecific antigen-binding molecule of any one of [1] to [1A], wherein the first antigen-binding moiety and the second antigen-binding moiety each comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).
[3] The multispecific antigen-binding molecule of any one of [1] or [2], wherein the first antigen-binding moiety and the second antigen-binding moiety each comprises an antibody variable region comprising any one of (a1) to (a17) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58; and
(a15) a heavy chain variable region comprising an amino acid sequence of SEQ ID NO: 81, and a light chain variable region comprising an amino acid sequence of SEQ ID NO: 60.
(a16) an antibody variable region that binds to the same epitope of any of the antibody variable region selected from (a1) to (a15); and
(a17) an antibody variable fragment that competes with the binding of any of the antibody variable fragment selected from (a1) to (a15).
[4] The multispecific antigen-binding molecule of any one of [1] to [3], wherein each of the first antigen-binding moiety and the second antigen-binding moiety is a Fab molecule and comprises at least one disulfide bond formed between the first antigen-binding moiety and the second antigen-binding moiety, preferably said at least one disulfide bond is formed between amino acid residues (cysteines) which are not in a hinge region, preferably between amino acid residues (cysteines) in the CH1 region of each antigen-binding moiety.
[4A] The multispecific antigen-binding molecule of [4], wherein each of the first antigen-binding moiety and the second antigen-binding moiety is a Fab molecule and comprises one disulfide bond formed between the amino acid residues (cysteines) at position 191 according to EU numbering in the respective CH1 region of the first antigen-binding moiety and the second antigen-binding moiety.
[5] The multispecific antigen-binding molecule of any one of [1] to [4A], wherein the third antigen binding moiety is fused to either one of the first antigen binding moiety or the second antigen binding moiety.
[5A] The multispecific antigen-binding molecule of [5], wherein the third antigen binding moiety is a Fab or scFv.
[6] The multispecific antigen-binding molecule of any one of [5] to [5A], wherein each of the first, second and third antigen binding moiety is a Fab molecule, wherein the third antigen binding moiety is fused at the C-terminus of the Fab heavy chain (CH1) to the N-terminus of the Fab heavy chain of either one of the first antigen binding moiety or the second antigen binding moiety, optionally via a peptide linker.
[6A] The multispecific antigen-binding molecule of any one of [5] to [6], wherein said peptide linker is selected from the group consisting of the amino acid sequence of SEQ ID NO: 248, SEQ ID NO: 249 or SEQ ID NO: 259.
[6B] The multispecific antigen-binding molecule of any one of [1] to [6A], wherein the first antigen binding moiety is identical to the second antigen binding moiety.
[7] The multispecific antigen-binding molecule of any one of [1] to [6B], wherein the third antigen binding moiety is a crossover Fab molecule in which the variable regions of the Fab light chain and the Fab heavy chain are exchanged, and wherein each of the first and second antigen binding moiety is a conventional Fab molecule.
[8] The multispecific antigen-binding molecule of [7], wherein in the constant domain CL of the light chain of each of the first and second antigen binding moiety, the amino acid(s) at position 123 and/or 124 is/are substituted independently by lysine (K), arginine (R) or histidine (H) (numbering according to Kabat), and wherein in the constant domain CH1 of the heavy chain of each of the first and second antigen binding moiety, the amino acid at position 147 and/or the amino acid at position 213 is substituted independently by glutamic acid (E) or aspartic acid (D) (numbering according to EU numbering).
[9] The multispecific antigen-binding molecule of [8], wherein in the constant domain CL of the light chain of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to EU numbering).
[10] The multispecific antigen-binding molecule of any one of [1] to [9], wherein the third antigen-binding moiety capable of binding to DLL3 comprises an antibody variable region comprising any one of (a1) to (a5) below:
(a1) the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239;
(a2) the heavy chain complementarity determining region (CDR) 1 of SEQ ID NO: 276, the heavy chain CDR 2 of SEQ ID NO: 277, the heavy chain CDR 3 of SEQ ID NO: 278, the light chain CDR 1 of SEQ ID NO: 279, the light chain CDR 2 of SEQ ID NO: 280 and the light chain CDR 3 of SEQ ID NO: 281;
(a3) the heavy chain complementarity determining region (CDR) 1 of SEQ ID NO: 285, the heavy chain CDR 2 of SEQ ID NO: 286, the heavy chain CDR 3 of SEQ ID NO: 287, the light chain CDR 1 of SEQ ID NO: 288, the light chain CDR 2 of SEQ ID NO: 289 and the light chain CDR 3 of SEQ ID NO: 290;
(a4) an antibody variable region that binds to the same epitope of any of the antibody variable region selected from (a1) to (a3); and
(a5) an antibody variable fragment that competes with the binding of any of the antibody variable fragment selected from (a1) to (a3).
[11] The multispecific antigen-binding molecule of any one of [1] to [10], wherein the third antigen-binding moiety capable of binding to DLL3 comprises an antibody variable region comprising any one of (a1) to (a6) below:
(a1) a heavy chain variable region comprising an amino acid sequence of SEQ ID NO: 232, and a light chain variable region comprising an amino acid sequence of SEQ ID NO: 236;
(a2) a heavy chain variable region comprising an amino acid sequence of SEQ ID NO: 264, and a light chain variable region comprising an amino acid sequence of SEQ ID NO: 265;
(a3) a heavy chain variable region comprising an amino acid sequence of SEQ ID NO: 266, and a light chain variable region comprising an amino acid sequence of SEQ ID NO: 267;
(a4) a heavy chain variable region comprising an amino acid sequence of SEQ ID NO: 268, and a light chain variable region comprising an amino acid sequence of SEQ ID NO: 269;
(a5) an antibody variable region that binds to the same epitope of any of the antibody variable region selected from (a1) to (a4); and
(a6) an antibody variable fragment that competes with the binding of any of the antibody variable fragment selected from (a1) to (a4).
[12] The multispecific antigen-binding molecule of any one of [1] to [11], further comprising a Fc domain.
[12A] The multispecific antigen-binding molecule of [12], wherein the Fc domain is composed of a first and a second Fc region subunit capable of stable association, and wherein the Fc domain exhibits reduced binding affinity to human Fc gamma receptor, as compared to a native human IgG1 Fc domain.
[12C] The multispecific antigen-binding molecule of any one of [12] to [12A], wherein the Fc domain exhibits enhanced FcRn-binding activity under an acidic pH condition (e.g., pH 5.8) as compared to that of an Fc region of a native IgG.
[12D] The multispecific antigen-binding molecule of [12C], wherein the Fc domain comprises Ala at position 434; Glu, Arg, Ser, or Lys at position 438; and Glu, Asp, or Gln at position 440, according to EU numbering.
[12E] The multispecific antigen-binding molecule of [12D], wherein the Fc domain comprises Ala at position 434; Arg or Lys at position 438; and Glu or Asp at position 440, according to EU numbering.
[12F] The multispecific antigen-binding molecule of [12E], wherein the Fc domain further comprises Ile or Leu at position 428; and/or Ile, Leu, Val, Thr, or Phe at position 436, according to EU numbering.
[12G] The multispecific antigen-binding molecule of any one of [12C] to [12F], wherein the Fc domain comprises a combination of amino acid substitutions selected from the group consisting of:
(a) N434A/Q438R/S440E;
(b) N434A/Q438R/S440D;
(c) N434A/Q438K/S440E;
(d) N434A/Q438K/S440D;
(e) N434A/Y436T/Q438R/S440E;
(f) N434A/Y436T/Q438R/S440D;
(g) N434A/Y436T/Q438K/S440E;
(h) N434A/Y436T/Q438K/S440D;
(i) N434A/Y436V/Q438R/S440E;
(j) N434A/Y436V/Q438R/S440D;
(k) N434A/Y436V/Q438K/S440E;
(l) N434A/Y436V/Q438K/S440D;
(m) N434A/R435H/F436T/Q438R/S440E;
(n) N434A/R435H/F436T/Q438R/S440D;
(o) N434A/R435H/F436T/Q438K/S440E;
(p) N434A/R435H/F436T/Q438K/S440D;
(q) N434A/R435H/F436V/Q438R/S440E;
(r) N434A/R435H/F436V/Q438R/S440D;
(s) N434A/R435H/F436V/Q438K/S440E;
(t) N434A/R435H/F436V/Q438K/S440D;
(u) M428L/N434A/Q438R/S440E;
(v) M428L/N434A/Q438R/S440D;
(w) M428L/N434A/Q438K/S440E;
(x) M428L/N434A/Q438K/S440D;
(y) M428L/N434A/Y436T/Q438R/S440E;
(z) M428L/N434A/Y436T/Q438R/S440D;
(aa) M428L/N434A/Y436T/Q438K/S440E;
(ab) M428L/N434A/Y436T/Q438K/S440D;
(ac) M428L/N434A/Y436V/Q438R/S440E;
(ad) M428L/N434A/Y436V/Q438R/S440D;
(ae) M428L/N434A/Y436V/Q438K/S440E;
(af) M428L/N434A/Y436V/Q438K/S440D;
(ag) L235R/G236R/S239K/M428L/N434A/Y436T/Q438R/S440E; and
(ah) L235R/G236R/A327G/A330S/P331S/M428L/N434A/Y436T/Q438R/S440E, according to EU numbering.
[12H] The multispecific antigen-binding molecule of any one of [12C] to [12G], wherein the Fc domain comprises a combination of amino acid substitutions of M428L/N434A/Q43 8R/S440E.
[12I] The multispecific antigen-binding molecule of any one of [12] to [12H], wherein the Fc domain is an IgG Fc domain, preferably a human IgG Fc domain, more preferably a human IgG1 Fc domain.
[12J] The multispecific antigen-binding molecule of any one of [12] to [12I], wherein the Fc domain comprises any of:
(a) a first Fc subunit comprising an amino acid sequence shown in SEQ ID NO: 100 and a second Fc subunit comprising an amino acid sequence shown in SEQ ID NO: 111; or
(b) a first Fc subunit comprising an amino acid sequence shown in SEQ ID NO: 99 and a second Fc subunit comprising an amino acid sequence shown in SEQ ID NO: 109.
[12K] The multispecific antigen-binding molecule of any one of [12] to [12J], wherein each of the first and second antigen-binding moiety is a Fab, wherein the first antigen-binding moiety is fused at the C-terminus of the Fab heavy chain to the N-terminus of the first or second subunit of the Fc domain, and the second antigen-binding moiety is fused at the C-terminus of the Fab heavy chain to the N-terminus of the remaining subunit of the Fc domain.
[12L] The multispecific antigen-binding molecule of [12K], wherein the third antigen binding moiety is fused at the C-terminus to the N-terminus of the Fab heavy chain of either one of the first antigen binding moiety or the second antigen binding moiety, optionally via a peptide linker.
[13] The multispecific antigen-binding molecule of any one of [1] to [12L], comprising five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NOs: 221 (chain 1), a polypeptide chain comprising an amino acid sequence of SEQ ID NO: 206 (chain 2), a polypeptide chain comprising an amino acid sequence of SEQ ID NOs: 211 (chain 3) and two polypeptide chains each comprising an amino acid sequence of SEQ ID NOs: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NO: 223 (chain 1), a polypeptide chain comprising an amino acid sequence of SEQ ID NO: 206 (chain 2), a polypeptide chain comprising an amino acid sequence of SEQ ID NO: 212 (chain 3) and two polypeptide chains each comprising an amino acid sequence of SEQ ID NO: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).

Another aspect of the present invention relates to:
[1] A method for (i) producing a preparation of a multispecific antigen binding molecule (that has been recombinantly produced by mammalian cells), (ii) purifying a multispecific antigen binding molecule having a desired conformation, or (iii) improving homogeneity of a preparation of a multispecific antigen binding molecule;
wherein the multispecific antigen binding molecule comprises a first antigen-binding moiety and a second antigen-binding moiety, each of the first antigen-binding moiety and the second antigen-binding moiety is a Fab and is capable of binding to a first antigen and a second antigen different from the first antigen, but does not bind both antigens at the same time;
wherein each of the first antigen-binding moiety and the second antigen-binding moiety comprises at least one cysteine residue (via mutation, substitution or insertion) which is not in a hinge region, preferably said at least one cysteine locates in the CH1 region; said at least one cysteine residue is capable of forming at least one disulfide bond between the first antigen-binding moiety and the second antigen-binding moiety, preferably in the CH1 region;
wherein said method comprises contacting the preparation with a reducing reagent.
[2] The method of [1], wherein each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.
[3] The method of [1] or [2], wherein said contacting the preparation with a reducing reagent allows and/or facilitates the formation of the at least one disulfide bond formed between amino acid residues located in the CH1 region or at the position 191 in the CH1 region (EU numbering).
[4] The method of [3], wherein said multispecific antigen binding molecule preparation (before contacting with the reducing agent) comprises two or more structural isoforms which differ by at least one disulfide bond formed between amino acid residues located in the CH1 region or at the position 191 in the CH1 region (EU numbering), and wherein the contacting with reducing agent preferentially enriches or increases the population of a structural isoform having at least one disulfide bond formed between amino acid residues located in the CH1 region or at the position 191 in the CH1 region (EU numbering).
[5] The method of any one of [1] to [4], wherein the pH of said reducing reagent contacting with the multispecific antigen binding molecule is from about 3 to about 10. [6] The method of [5], wherein the pH of said reducing reagent contacting with the multispecific antigen binding molecule is about 6, 7 or 8.
[7] The method of [6], wherein the pH of said reducing reagent contacting with the multispecific antigen binding molecule is about 7.
[8] The method of [5], wherein the pH of said reducing reagent contacting with the multispecific antigen binding molecule is about 3.
[9] The method of any one of [1] to [8], wherein the reducing agent is selected from the group consisting of TCEP, 2-MEA, DTT, Cysteine, GSH and Na₂SO₃.
[10] The method of [9], wherein the reducing agent is TCEP, preferably 0.25 mM TCEP.
[11] The method of any one of [1] to [9], wherein the concentration of the reducing agent is from about 0.01 mM to about 100 mM.
[12] The method of [11], wherein the concentration of the reducing agent is about 0.01, 0.05, 0.1, 0.25, 0.5, 1, 2.5, 5, 10, 25, 50, 100 mM, preferably about 0.25 mM.
[13] The method of any one of [1] to [12], wherein the contacting step is performed for at least 30 minutes.
[14] The method of any one of [1] to [12], wherein the contacting step is performed for about 10 minutes to about 48 hours.
[15] The method of any one of [1] to [12], wherein the contacting step is performed for about 2 hours or about 18 hours.
[16] The method of any one of [1] to [15], wherein the contacting step is performed at a temperature of about 4 degrees Celsius to 37 degrees Celsius, preferably at 23 degrees Celsius to 25 degrees Celsius.
[17] The method of any one of [1] to [16], wherein said multispecific antigen binding molecule is at least partially purified prior to said contacting step with the reducing agent.
[18] The method of [17], wherein said multispecific antigen binding molecule is partially purified by affinity chromatography (preferably Protein A chromatography) prior to said contacting.
[19] The method of any one of [1] to [18], wherein the concentration of the multispecific antigen binding molecule is from about 0.1 mg/ml to about 50 mg/ml or more.
[20] The method of [19], wherein the concentration of the multispecific antigen binding molecule is about 10 mg/ml or about 20 mg/ml.
[21] The method of any one of [1] to [20], further comprising a step of promoting re-oxidization of cysteine disulfide bonds, preferably by removing the reducing agent, preferably by dialysis or buffer exchange.
[22] The method of any one of [1] to [21], wherein each of the first antigen-binding moiety and the second antigen-binding moiety is capable of binding to CD3 and CD137 but does not bind both CD3 and CD137 at the same time.
[23] The method of [22], wherein the first antigen-binding moiety and the second antigen-binding moiety each comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).
[24] The method of [23], wherein the first antigen-binding moiety and the second antigen-binding moiety each comprises an antibody variable region comprising any one of (a1) to (a17) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58; and
(a15) a heavy chain variable region comprising an amino acid sequence of SEQ ID NO: 81, and a light chain variable region comprising an amino acid sequence of SEQ ID NO: 60.
(a16) an antibody variable region that binds to the same epitope of any of the antibody variable region selected from (a1) to (a15); and
(a17) an antibody variable fragment that competes with the binding of any of the antibody variable fragment selected from (a1) to (a15).
[25] The method of any one of [1] to [24], wherein the multispecific antigen binding molecule further comprises a third antigen-binding moiety capable of binding to a third antigen different from the first and the second antigen, preferably an antigen expressed on a cancer cell/tissue.
[26] The multispecific antigen-binding molecule of [25], wherein the third antigen binding moiety is fused to either one of the first antigen binding moiety or the second antigen binding moiety.
[27] The method of any one of [25] to [26], wherein the third antigen binding moiety is a Fab or scFv.
[28] The method of any one of [25] to [27], wherein each of the first, second and third antigen binding moiety is a Fab molecule, wherein the third antigen binding moiety is fused at the C-terminus of the Fab heavy chain (CH1) to the N-terminus of the Fab heavy chain of either one of the first antigen binding moiety or the second antigen binding moiety, optionally via a peptide linker.
[29] The method of [28], wherein said peptide linker is selected from the group consisting of the amino acid sequence of SEQ ID NO: 248, SEQ ID NO: 249 or SEQ ID NO: 259.
[30] The method of any one of [1] to [29], wherein the first antigen binding moiety is identical to the second antigen binding moiety.
[30A] The method of any one of [25] to [30], wherein the third antigen binding moiety is a crossover Fab molecule in which the variable regions of the Fab light chain and the Fab heavy chain are exchanged, and wherein each of the first and second antigen binding moiety is a conventional Fab molecule.
[30B] The method of any one of [25] to [30A], wherein in the constant domain CL of the light chain of each of the first and second antigen binding moiety, the amino acid(s) at position 123 and/or 124 is/are substituted independently by lysine (K), arginine (R) or histidine (H) (numbering according to Kabat), and wherein in the constant domain CH1 of the heavy chain of each of the first and second antigen binding moiety, the amino acid at position 147 and/or the amino acid at position 213 is substituted independently by glutamic acid (E) or aspartic acid (D) (numbering according to EU numbering).
[30C] The method of [30B], wherein in the constant domain CL of the light chain of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to EU numbering).
[31] The method of any one of [1] to [30], wherein the third antigen-binding moiety is capable of binding to DLL3, preferably human DLL3.
[32] The method of [31], wherein the third antigen-binding moiety capable of binding to DLL3 comprises an antibody variable region comprising the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239.
[33] The method of [32], wherein the third antigen-binding moiety capable of binding to DLL3 comprises an antibody variable region comprising: a heavy chain variable region comprising an amino acid sequence of SEQ ID NO: 232, and a light chain variable region comprising an amino acid sequence of SEQ ID NO: 236.
[34] The method of any one of [1] to [33], wherein the multispecific antigen binding molecule further comprises a Fc domain.
[35] The method of [34], wherein the Fc domain is composed of a first and a second Fc region subunit capable of stable association, and wherein the Fc domain exhibits reduced binding affinity to human Fc gamma receptor, as compared to a native human IgG1 Fc domain.
[36] The method of any one of [1] to [35], wherein the multispecific antigen binding molecule comprises five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NO: 221 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NO: 223 (chain 1), a polypeptide chain comprising an amino acid sequence of SEQ ID NO: 206 (chain 2), a polypeptide chain comprising an amino acid sequence of SEQ ID NO: 212 (chain 3) and two polypeptide chains each comprising an amino acid sequence of SEQ ID NO: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).
[37] The method of any one of [1] to [36], wherein the fourth polypeptide (chain 4) and the fifth polypeptide (chain 5) are identical.
[38] A preparation of a multispecific antigen binding molecule prepared according to the method of any one of [1] to [37], said preparation having a homogeneous population of said multispecific antigen binding molecule having at least one disulfide bond in the CH1 region (position 191 according to EU numbering).
[39] A preparation of a multispecific antigen binding molecule prepared according to the method of any one of [1] to [37], said preparation having at least 50%, 60%, 70%, 80%, 90%, preferably at least 95% molar ratio of said multispecific antigen binding molecule having at least one disulfide bond in the CH1 region (position 191 according to EU numbering).

Yet another aspect of the present invention relates to:
[1] A method for producing a multispecific antigen-binding molecule, wherein said multispecific antigen-binding molecule comprises:
a first antigen-binding moiety and a second antigen-binding moiety; wherein each of the first antigen-binding moiety and the second antigen-binding is a Fab and is capable of binding to a first antigen and a second antigen different from the first antigen, but does not bind both antigens at the same time; and
a third antigen-binding moiety comprising a heavy chain variable region (VH) and a light chain variable region (VL), which is capable of binding to a third antigen different from the first and the second antigen, preferably an antigen expressed on a cancer cell/tissue,
said method comprising:
(a) providing one or more nucleic acid(s) encoding:
i. a first polypeptide comprising (starting from N-terminus to C-terminus) the VH or VL of the third antigen-binding moiety, optionally a heavy chain constant region (CH1); and the VH or VL of the first antigen-binding moiety, a heavy chain constant region (CH1); and optionally a hinge region and/or a Fc region (CH2 and CH3);
ii. a second polypeptide comprising (starting from N-terminus to C-terminus) the VH or VL of the third antigen-binding moiety, optionally a light chain constant region (CL);
iii. a third polypeptide comprising (starting from N-terminus to C-terminus) a VH or VL of the second antigen-binding moiety, a heavy chain constant region (CH1); and optionally a hinge region and/or a Fc region (CH2 and CH3);
iv. a fourth polypeptide comprising (starting from N-terminus to C-terminus) a VH or VL of the second antigen-binding moiety, optionally a light chain constant region (CL); and
v. a fifth polypeptide comprising (starting from N-terminus to C-terminus) a VH or VL of the first antigen-binding moiety, optionally a light chain constant region (CL)
(b) introducing the one or more nucleic acid(s) produced in (a) into a host cell;
(c) culturing the host cell such that the polypeptides in (i) to (v) are expressed; and
(d) collecting the multispecific antigen-binding molecule comprising the five polypeptides in (i) to (v) from the culture solution of the cell cultured in step (c); and
wherein optionally the polypeptides in (iv) to (v) are identical; and
wherein each of the first antigen-binding moiety and the second antigen-binding moiety comprises at least one cysteine residue (via mutation, substitution or insertion) which is not in a hinge region, preferably said at least one cysteine locates in the CH1 region; said at least one cysteine residue is capable of forming at least one disulfide bond between the first antigen-binding moiety and the second antigen-binding moiety, preferably in the CH1 region;
wherein said method comprises contacting the preparation with a reducing reagent.
[2] The method of [1], wherein each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.
[3] The method of any one of [1] to [2], further comprising step (e) contacting the multispecific antigen-binding molecule (multispecific antigen binding molecule) preparation collected from step (d) with a reducing reagent under reducing conditions which allow the cysteine(s) in the CH1 region (position 191 according to EU numbering) to form one or more disulfide bond.
[4] The method of [3], wherein said multispecific antigen binding molecule preparation collected from step (d) (before contacting with the reducing agent) comprises two or more structural isoforms which differ by at least one disulfide bond formed between amino acid residues located in the CH1 region or at the position 191 in the CH1 region (EU numbering), and the step (e) contacting with reducing agent preferentially enriches or increases the population of a multispecific antigen binding molecule structural isoform having at least one disulfide bond formed between amino acid residues located in the CH1 region or at the position 191 in the CH1 region (EU numbering).
[5] The method of any one of [3] to [4], wherein the pH of said reducing reagent contacting with the multispecific antigen binding molecule is from about 3 to about 10.
[6] The method of [5], wherein the pH of said reducing reagent contacting with the multispecific antigen binding molecule is about 6, 7 or 8.
[7] The method of [6], wherein the pH of said reducing reagent contacting with the multispecific antigen binding molecule is about 7.
[8] The method of [5], wherein the pH of said reducing reagent contacting with the multispecific antigen binding molecule is about 3.
[9] The method of any one of [3] to [8], wherein the reducing agent is selected from the group consisting of TCEP, 2-MEA, DTT, Cysteine, GSH and Na₂SO₃.
[10] The method of [9], wherein the reducing agent is TCEP, preferably 0.25 mM TCEP.
[11] The method of any one of [3] to [9], wherein the concentration of the reducing agent is from about 0.01 mM to about 100 mM.
[12] The method of [11], wherein the concentration of the reducing agent is about 0.01, 0.05, 0.1, 0.25, 0.5, 1, 2.5, 5, 10, 25, 50, 100 mM, preferably about 0.25 mM.
[13] The method of any one of [3] to [12], wherein the contacting step is performed for at least 30 minutes.
[14] The method of any one of [3] to [12], wherein the contacting step is performed for about 10 minutes to about 48 hours.
[15] The method of any one of [3] to [12], wherein the contacting step is performed for about 2 hours or about 18 hours.
[16] The method of any one of [3] to [15], wherein the contacting step is performed at a temperature of about 4 degrees Celsius to 37 degrees Celsius, preferably at 23 degrees Celsius to 25 degrees Celsius.
[17] The method of any one of [3] to [16], wherein said multispecific antigen binding molecule is at least partially purified prior to said contacting step with the reducing agent.
[18] The method of [17], wherein said multispecific antigen binding molecule is partially purified by affinity chromatography (preferably Protein A chromatography) prior to said contacting.
[19] The method of any one of [3] to [18], wherein the concentration of the multispecific antigen binding molecule is from about 0.1 mg/ml to about 50 mg/ml or more.
[20] The method of [19], wherein the concentration of the multispecific antigen binding molecule is about 10 mg/ml or about 20 mg/ml.
[21] The method of any one of [3] to [20], further comprising a step of promoting re-oxidization of cysteine disulfide bonds, preferably by removing the reducing agent, preferably by dialysis or buffer exchange.
[22] A preparation of a multispecific antigen binding molecule prepared according to the method of any one of [3] to [21], said preparation having a homogeneous population of said multispecific antigen binding molecule having at least one disulfide bond in the CH1 region (position 191 according to EU numbering).
[23] A preparation of a multispecific antigen binding molecule prepared according to the method of any one of [3] to [21], said preparation having at least 50%, 60%, 70%, 80%, 90%, preferably at least 95% molar ratio of said multispecific antigen binding molecule having at least one disulfide bond in the CH1 region (position 191 according to EU numbering).
[24] The method of any one of [1] to [21], wherein the third antigen-binding moiety is a conventional Fab, and wherein
(a) the first polypeptide comprising (starting from N-terminus to C-terminus) the VH of the third antigen-binding moiety, a heavy chain constant region (CH1); and the VH of the first antigen-binding moiety, a heavy chain constant region (CH1); and optionally a hinge region and/or a Fc region (CH2 and CH3);
(b) a second polypeptide comprising (starting from N-terminus to C-terminus) the VL of the third antigen-binding moiety, and a light chain constant region (CL);
(c) a third polypeptide comprising (starting from N-terminus to C-terminus) a VH of the second antigen-binding moiety, a heavy chain constant region (CH1); and optionally a hinge region and/or a Fc region (CH2 and CH3);
(d) a fourth polypeptide comprising (starting from N-terminus to C-terminus) a VL of the second antigen-binding moiety, and a light chain constant region (CL); and
(e) a fifth polypeptide comprising (starting from N-terminus to C-terminus) a VL of the first antigen-binding moiety, and a light chain constant region (CL).
[25] The method of any one of [1] to [21], wherein the third antigen-binding moiety is a VH/VL crossover Fab (in which the variable regions of the Fab light chain and the Fab heavy chain are exchanged), and wherein
(a) the first polypeptide comprising (starting from N-terminus to C-terminus) the VL of the third antigen-binding moiety, a heavy chain constant region (CH1); and the VH of the first antigen-binding moiety, a heavy chain constant region (CH1); and optionally a hinge region and/or a Fc region (CH2 and CH3);
(b) a second polypeptide comprising (starting from N-terminus to C-terminus) the VH of the third antigen-binding moiety, and a light chain constant region (CL);
(c) a third polypeptide comprising (starting from N-terminus to C-terminus) a VH of the second antigen-binding moiety, a heavy chain constant region (CH1); and optionally a hinge region and/or a Fc region (CH2 and CH3);
(d) a fourth polypeptide comprising (starting from N-terminus to C-terminus) a VL of the second antigen-binding moiety, and a light chain constant region (CL); and
(e) a fifth polypeptide comprising (starting from N-terminus to C-terminus) a VL of the first antigen-binding moiety, and a light chain constant region (CL).
[26] The method of [25], wherein in the CL of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to EU numbering).
[26-2] The method of any one of [1] to [21], wherein in step (a)(i), the first polypeptide, between the third antigen-binding moiety and the VH or VL of the first antigen-binding moiety, further comprises a peptide linker.
[27] The method of [26-2], wherein said peptide linker is selected from the group consisting of the amino acid sequence of SEQ ID NO: 248, SEQ ID NO: 249 or SEQ ID NO: 259.
[28] The method of any one of [1] to [27], wherein each of the first antigen-binding moiety and the second antigen-binding moiety is capable of binding to CD3 and CD137 but does not bind both CD3 and CD137 at the same time.
[29] The method of [28], wherein the first antigen-binding moiety and the second antigen-binding moiety each comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).
[30] The method of [29], wherein the first antigen-binding moiety and the second antigen-binding moiety each comprises an antibody variable region comprising any one of (a1) to (a17) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58; and
(a15) a heavy chain variable region comprising an amino acid sequence of SEQ ID NO: 81, and a light chain variable region comprising an amino acid sequence of SEQ ID NO: 60.
(a16) an antibody variable region that binds to the same epitope of any of the antibody variable region selected from (a1) to (a15); and
(a17) an antibody variable fragment that competes with the binding of any of the antibody variable fragment selected from (a1) to (a15).
[31] The method of any one of [1] to [30], wherein the third antigen-binding moiety is capable of binding to DLL3, preferably human DLL3.
[32] The method of [31], wherein the third antigen-binding moiety capable of binding to DLL3 comprises an antibody variable region comprising the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239.
[33] The method of [32], wherein the third antigen-binding moiety capable of binding to DLL3 comprises an antibody variable region comprising: a heavy chain variable region comprising an amino acid sequence of SEQ ID NO: 232, and a light chain variable region comprising an amino acid sequence of SEQ ID NO: 236.
[34] The method of any one of [1] to [33], wherein the multispecific antigen binding molecule further comprises a Fc domain.
[35] The method of [34], wherein the Fc domain is composed of a first and a second Fc region subunit capable of stable association, and wherein the Fc domain exhibits reduced binding affinity to human Fc gamma receptor, as compared to a native human IgG1 Fc domain.
[36] The method of any one of [1] to [35], wherein the multispecific antigen binding molecule comprises five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NO: 221 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NOs: 223 (chain 1), a polypeptide chain comprising an amino acid sequence of SEQ ID NO: 206 (chain 2), a polypeptide chain comprising an amino acid sequence of SEQ ID NOs: 212 (chain 3) and two polypeptide chains each comprising an amino acid sequence of SEQ ID NOs: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).
[37] The method of any one of [1] to [36], wherein the fourth polypeptide (chain 4) and the fifth polypeptide (chain 5) are identical.
[38] The method of any one of [1] or [37], wherein only one nucleic acid, or two, three, four or five different nucleic acids encode and express the first, second, third, fourth and fifth polypeptides.

In yet another aspect, the present invention relates to:
[1] A method for capturing and/or removing a target antibody from an antibody preparation, comprising the steps of:
a) contacting the antibody preparation comprising the target antibody with an antigen-binding molecule immobilized on a support; and
b) allowing capture of the target antibody by specific binding to the antigen-binding molecule;
wherein said antibody comprises at least two Fabs from an IgG (preferably human IgG or human IgG1), and said antibody preparation comprises two antibody structural isoforms which differ by a disulfide bond formed between the two Fabs at the CH1 domain; and
wherein said antigen-binding molecule specifically binds and captures the target antibody which does not comprise the disulfide bond.
[2] The method of [1], wherein said antigen-binding molecule binds to the target antibody at an epitope which is only accessible to the antigen-binding molecule when the target antibody does not have the disulfide bond.
[3] The method of [1] or [2], wherein said disulfide bond is a disulfide bond formed between the two Fabs of the antibody at position 191 according to EU numbering in the CH1 domain.
[4] The method of any one of [1] to [3], wherein said antigen-binding molecule is an antibody which comprises any one selected from the group consisting of the following:
(a1) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 182, the light chain CDR 2 of SEQ ID NO: 186 and the light chain CDR 3 of SEQ ID NO: 190;
(a2) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 183, the light chain CDR 2 of SEQ ID NO: 187 and the light chain CDR 3 of SEQ ID NO: 191;
(a3) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 184, the light chain CDR 2 of SEQ ID NO: 188 and the light chain CDR 3 of SEQ ID NO: 192;
(a4) the heavy chain CDR 1 of SEQ ID NO: 169, the heavy chain CDR 2 of SEQ ID NO: 173, the heavy chain CDR 3 of SEQ ID NO: 177, the light chain CDR 1 of SEQ ID NO: 185, the light chain CDR 2 of SEQ ID NO: 189 and the light chain CDR 3 of SEQ ID NO: 193;
(a5) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 115, the light chain CDR 2 of SEQ ID NO: 124 and the light chain CDR 3 of SEQ ID NO: 134;
(a6) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 116, the light chain CDR 2 of SEQ ID NO: 125 and the light chain CDR 3 of SEQ ID NO: 135;
(a7) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 118, the light chain CDR 2 of SEQ ID NO: 128 and the light chain CDR 3 of SEQ ID NO: 137;
(a8) an antibody that binds to the same epitope of the antibody comprising any one of (a1) to (a7); and
(a9) an antibody that competes with the binding of the antibody comprising any one of (a1) to (a7).
[5] The method of any one of [1] to [3], wherein said antigen-binding molecule is an antibody which comprises any one selected from the group consisting of the following:
(a1) a heavy chain variable region comprising an amino acid sequence of SEQ ID NO: 162, and a light chain variable region comprising an amino acid sequence of SEQ ID NO: 178;
(a2) a heavy chain variable region comprising an amino acid sequence of SEQ ID NO: 163, and a light chain variable region comprising an amino acid sequence of SEQ ID NO: 179;
(a3) a heavy chain variable region comprising an amino acid sequence of SEQ ID NO: 164, and a light chain variable region comprising an amino acid sequence of SEQ ID NO: 180;
(a4) a heavy chain variable region comprising an amino acid sequence of SEQ ID NO: 165, and a light chain variable region comprising an amino acid sequence of SEQ ID NO: 181;
(a5) a heavy chain variable region comprising an amino acid sequence of SEQ ID NO: 162, and a light chain variable region comprising an amino acid sequence of SEQ ID NO: 196;
(a6) a heavy chain variable region comprising an amino acid sequence of SEQ ID NO: 163, and a light chain variable region comprising an amino acid sequence of SEQ ID NO: 197;
(a7) a heavy chain variable region comprising an amino acid sequence of SEQ ID NO: 164, and a light chain variable region comprising an amino acid sequence of SEQ ID NO: 198;
(a8) an antibody that binds to the same epitope of the antibody comprising any one of (a1) to (a7); and
(a9) an antibody that competes with the binding of the antibody comprising any one of (a1) to (a7).
[5A] The method of any one of [1] to [5], wherein the target antibody comprises five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NOs: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NO: 221 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NO: 223 (chain 1), a polypeptide chain comprising an amino acid sequence of SEQ ID NO: 206 (chain 2), a polypeptide chain comprising an amino acid sequence of SEQ ID NO: 212 (chain 3) and two polypeptide chains each comprising an amino acid sequence of SEQ ID NO: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).
[6] An antigen-binding molecule which comprises any one selected from the group consisting of the following:
(a1) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 182, the light chain CDR 2 of SEQ ID NO: 186 and the light chain CDR 3 of SEQ ID NO: 190;
(a2) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 183, the light chain CDR 2 of SEQ ID NO: 187 and the light chain CDR 3 of SEQ ID NO: 191;
(a3) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 184, the light chain CDR 2 of SEQ ID NO: 188 and the light chain CDR 3 of SEQ ID NO: 192;
(a4) the heavy chain CDR 1 of SEQ ID NO: 169, the heavy chain CDR 2 of SEQ ID NO: 173, the heavy chain CDR 3 of SEQ ID NO: 177, the light chain CDR 1 of SEQ ID NO: 185, the light chain CDR 2 of SEQ ID NO: 189 and the light chain CDR 3 of SEQ ID NO: 193;
(a5) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 115, the light chain CDR 2 of SEQ ID NO: 124 and the light chain CDR 3 of SEQ ID NO: 134;
(a6) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 116, the light chain CDR 2 of SEQ ID NO: 125 and the light chain CDR 3 of SEQ ID NO: 135;
(a7) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 118, the light chain CDR 2 of SEQ ID NO: 128 and the light chain CDR 3 of SEQ ID NO: 137;
(a8) an antibody that binds to the same epitope of the antibody comprising any one of (a1) to (a7); and
(a9) an antibody that competes with the binding of the antibody comprising any one of (a1) to (a7).
[7] An antigen-binding molecule which comprises any one selected from the group consisting of the following:
(a1) a heavy chain variable region comprising an amino acid sequence of SEQ ID NO: 162, and a light chain variable region comprising an amino acid sequence of SEQ ID NO: 178;
(a2) a heavy chain variable region comprising an amino acid sequence of SEQ ID NO: 163, and a light chain variable region comprising an amino acid sequence of SEQ ID NO: 179;
(a3) a heavy chain variable region comprising an amino acid sequence of SEQ ID NO: 164, and a light chain variable region comprising an amino acid sequence of SEQ ID NO: 180;
(a4) a heavy chain variable region comprising an amino acid sequence of SEQ ID NO: 165, and a light chain variable region comprising an amino acid sequence of SEQ ID NO: 181;
(a5) a heavy chain variable region comprising an amino acid sequence of SEQ ID NO: 162, and a light chain variable region comprising an amino acid sequence of SEQ ID NO: 196;
(a6) a heavy chain variable region comprising an amino acid sequence of SEQ ID NO: 163, and a light chain variable region comprising an amino acid sequence of SEQ ID NO: 197;
(a7) a heavy chain variable region comprising an amino acid sequence of SEQ ID NO: 164, and a light chain variable region comprising an amino acid sequence of SEQ ID NO: 198;
(a8) an antibody that binds to the same epitope of the antibody comprising any one of (a1) to (a7); and
(a9) an antibody that competes with the binding of the antibody comprising any one of (a1) to (a7).
[8] The antigen-binding molecule of [6] or [7], which specifically binds to CH1 of human IgG1.
[9] The antigen-binding molecule of [8], which does not specifically bind to CH1 of human IgG1 when a disulfide bond is formed between the CH1 domains of the two Fabs of human IgG1.
[10] The antigen-binding molecule of [9], wherein said disulfide bond is a disulfide bond formed between the two Fabs of the IgG1 at position 191 according to EU numbering in the CH1 domain.
[11] The antigen-binding molecule of any one of [8] to [10], which does not bind to CH1 of human IgG4.
[12] Use of the antigen-binding molecule of any one of [6] to [11] in purification, analytical or quantification of an antibody sample.

### BRIEF DESCRIPTION OF DRAWINGS

FIG. 1 illustrates various antibody formats. Annotation of each Fv region for Table 2. Diagrams depicting (a) (1+2) trivalent antibody applied with LINC technology, name 1+2 Dual/LINC (“LINC” means the engineered disulfide bond at e.g. CH1 region); and (b) (1+2) format trivalent antibody without the engineered disulfide bond.

FIG. 2a is an illustration to depict that LINC-Ig technology in 1+2 format can reduce toxicity. LINC-Ig (comprises “LINC”, i.e. the engineered disulfide bond at e.g. CH1 region) can restrict antigen binding of the antibody shown in FIG. 1(a) primarily to cis mode i.e. binding to antigens present on the same immune cell. In contrast, (1+2) trivalent format without the engineered disulfide bond shown in FIG. 1(b) could result in trans antigen-binding mode i.e. binding of the antibody of FIG. 1(b) to antigens present on two different immune cells. This may cause cross-linking of two immune cells independent of tumor antigen binding which could increase toxicity.

FIG. 2b is a schematic illustration to depict that (1+2) trivalent antibody without the engineered disulfide bond in FIG. 1(b) with unpaired surface cysteines could form disulfide bond with a molecule that contains free thiol group e.g. free cysteine or glutathione in the antibody preparation, which leads to capping of unpaired cysteines on the antibody which prevents LINC formation (Left). Treating such capped antibodies with reducing agents can help de-cap the surface cysteines (Middle), and further re-oxidation (e.g. remove reducing reagent via buffer exchange) of de-capped antibody promotes disulfide bond formation between the de-capped cysteines to facilitate LINC formation (Right). (For simplicity, the native disulfide linkages e.g. between hinge regions and between heavy chain CH1 and light chain CL of the antibody are not shown).

FIG. 3 shows non-reducing SDS-PAGE analysis of trivalent (1+2) Ab with and without LINC engineering (with or without the S191C mutation for engineered disulfide bond formation). A single protein migration band for the (1+2) trivalent format without introduction of the S191C mutations (lanes 2 & 5) was observed. Whereas two protein migration bands were detected for the (1+2) Dual/LINC antibody variants, the slower migration band showed similar electrophoretic mobility as the (1+2) trivalent format without introduction of the LINC mutations. This suggests that the faster migration band is the Dual/LINC-Ig. Percentage of Dual-LINC-Ig with unpaired cysteines (unLINC format) in the antibody sample can be calculated by intensity of slower band/upper band corresponding to “UnLINC” format divided by the intensity sum of two bands correspond to “LINC” and “UnLINC” structure.

FIG. 4 shows non-reducing SDS-PAGE of Dual-LINC-Ig after treatment with different reducing agents. “−” represents “No CuSO₄ addition”. “+” represents addition of 25 micromolar/50 micromolar CuSO₄ during overnight (O/N) re-oxidation.

FIG. 5 shows non-reducing SDS-PAGE of Dual-LINC-Ig after TCEP treatment with different concentrations of Dual-LINC-Ig.

FIG. 6 shows non-reducing SDS-PAGE of Dual-LINC-Ig after TCEP treatment with different incubation periods. Percentage of Dual-LINC-Ig with unpaired cysteines (unLINC format) in the antibody sample can be calculated by intensity of slower band/upper band corresponding to “UnLINC” format divided by the sum of two bands correspond to “LINC” and “UnLINC” structure.

FIG. 7 is a schematic diagram showing concept of conformation-specific antibody (e.g. a conformational specific anti-CH1 antibody) which only binds to the target antibody (e.g. an epitope within the CH1 region) when the antibody does not have engineered disulfide bond e.g. at CH1 region (“unpaired cysteines” form), wherein the epitope(s) is/are not accessible to the conformation-specific antibody when the target antibody has engineered disulfide bond (“paired cysteine” form) due to e.g. steric hindrance or reduced distance between the two Fabs caused by the engineered disulfide bond.

FIG. 8 illustrates the Dual/LINC (1+2) antibody format comprising three Fabs, wherein two of the Fabs (Fab B and C, comprised in Chain 1-Chain 5 and Chain 3-Chain 4 respectively) each comprises an engineered cysteine (capable of forming engineered disulfide bond linking both Fabs, and hence can exist in either “unpaired cysteines” form or “paired cysteines” form) and one Fab (Fab A, comprised in Chain 1-Chain 2) which does not comprise engineered cysteine (only exists in “paired cysteines” form). (a) the CH1 for Fab A is in “unpaired cysteines” or “unLINC” form/conformation, and the CH1 of Fab B and Fab C are in “paired cysteines” or “LINC” form/conformation (b) Conformation-specific anti-IgG1 CH1 antibody can only bind to the CH1 of IgG1 in “unpaired cysteines” or “unLINC” form/conformation. The CH1 of Fab A was engineered to have IgG4 CH1 sequence. As a result, the conformation-specific anti-IgG1 CH1 antibody will only bind to Dual/LINC (1+2) antibody in the “unpaired cysteines” or “unLINC” form/conformation, but not the antibody species with “paired cysteines” or “LINC” form/conformation.

FIG. 9a is an illustration of various tool antibodies having different antibody formats for screening of conformation-specific anti-CH1 antibodies.

FIG. 9b shows amino acid sequence SEQ ID NOs for each of the polypeptide chains of the tool antibodies.

FIG. 10a shows chromatography profile for affinity purification of DLL3-DualAE05/DualAE05-FF056 using conformation-specific anti-CH1 antibody FAB0133Hh/FAB0133L0001 affinity column.

FIG. 10b shows non-reducing SDS-PAGE analysis of the eluted antibodies in the affinity purification of DLL3-DualAE05/DualAE05-FF056 using conformation-specific anti-CH1 antibody FAB0133Hh/FAB0133L0001 affinity column. Specifically, the flow-through fractions comprise high purity of DualAE05/DualAE05-FF056 which is in “paired cysteines” or “LINC” form (flowthrough: white bar) as indicated by one predominant protein band which migrates faster in the non-reducing SDS-PAGE analysis (Lanes 1 to 13); wash fractions comprise mixture of DualAE05/DualAE05-FF056 which is in “unpaired cysteines” form and DualAE05/DualAE05-FF056 which is in “paired cysteines” form (wash: gray bar); and eluted fractions comprise predominantly DualAE05/DualAE05-FF056 which is in “unpaired cysteines” or “unLINC” form (50 mM HCl acid elution: black bar) as indicated by one predominant protein band which migrates slower in the non-reducing SDS-PAGE analysis (Lanes 20 to 23). Purity of the antibody sample was determined by band densitometry of non-reducing SDS-PAGE. The image of unstained gel shown here was captured by ChemiDoc Imaging Systems (Bio-Rad) and densitometry analysis of protein bands of unLINC and LINC form of LINC-Ig antibodies was performed using Image Lab Software (Bio-Rad). The unLINC form migrated slightly slower than LINC form due to conformational difference. The protein sample containing 30-40% of unLINC form (indicated as INPUT) was applied to anti-CH1 column in order to obtain higher purity of antibodies.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The definitions and detailed description below are provided to facilitate understanding of the present disclosure illustrated herein.

### Definitions

Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

Alteration of Amino Acids

For amino acid alteration (also described as “amino acid substitution” or “amino acid mutation” within this description) in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

Polypeptides

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Recombinant Methods and Compositions

Antibodies and antigen-binding molecules may be produced using recombinant methods and compositions, e.g., as described in U.S. Pat. No. 4,816,567. In one embodiment, isolated nucleic acid encoding an antibody as described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making the multispecific antigen-binding molecule of the present invention is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody described herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Recombinant production of an antigen-binding molecule described herein could be done with methods similar to those described above, by using a host cell comprises (e.g., has been transformed with) one or plural vectors comprising nucleic acid that encodes an amino acid sequence comprising the whole antigen-binding molecule or part of the antigen-binding molecule.

Antigen-Binding Molecule and Multispecific Antigen-Binding Molecules

The term “antigen-binding molecule”, as used herein, refers to any molecule that comprises an antigen-binding site or any molecule that has binding activity to an antigen, and may further refers to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such. Scaffold molecules comprising known stable conformational structure such as alpha/beta barrel as scaffold, and in which part of the molecule is made into antigen-binding site, is also one embodiment of the antigen binding molecule described herein.

“Multispecific antigen-binding molecules” refers to antigen-binding molecules that bind specifically to more than one antigen. The term “bispecific” means that the antigen binding molecule is able to specifically bind to at least two distinct antigenic determinants. The term “trispecific” means that the antigen binding molecule is able to specifically bind to at least three distinct antigenic determinants. In certain embodiments, the multispecific antigen binding molecule of the present application is a trispecific antigen binding molecule, i.e. it is capable of specifically binding to three different antigen-capable of binding to either one of CD3 or CD137 but does not bind to both antigens simultaneously, and is capable of specifically binding to DLL3.

In an aspect, the present disclosure provides a multispecific antigen binding molecule comprising:

a first antigen-binding moiety and a second antigen-binding moiety, each of which is capable of binding to CD3 and CD137, but does not bind to CD3 and CD137 at the same time; and

a third antigen-binding moiety that is capable of binding to a third antigen, preferably an antigen expressed on a cancer cell/tissue.

In an aspect, the present disclosure provides a multispecific antigen-binding molecule comprising:

a first antigen-binding moiety and a second antigen-binding moiety, each of which is capable of binding to CD3 and CD137, but does not bind to CD3 and CD137 at the same time; and

a third antigen-binding moiety that is capable of binding to DLL3, preferably human DLL3.

In an aspect, the first antigen-binding moiety and the second antigen-binding moiety each comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

In an aspect, the first antigen-binding moiety and the second antigen-binding moiety each comprises an antibody variable region comprising any one of (a1) to (a17) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58; and
(a15) a heavy chain variable region comprising an amino acid sequence of SEQ ID NO: 81, and a light chain variable region comprising an amino acid sequence of SEQ ID NO: 60.
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

In an aspect, each of the first antigen-binding moiety and the second antigen-binding moiety is a Fab molecule and comprises at least one disulfide bond formed between the first antigen-binding moiety and the second antigen-binding moiety, preferably the at least one disulfide bond is formed between amino acid residues (cysteines) which are not in a hinge region, preferably between amino acid residues (cysteines) in the CH1 region of each antigen-binding moiety.

In an aspect, each of the first antigen-binding moiety and the second antigen-binding moiety is a Fab molecule and comprises one disulfide bond formed between the amino acid residues (cysteines) at position 191 according to EU numbering in the respective CH1 region of the first antigen-binding moiety and the second antigen-binding moiety.

In an aspect, the third antigen binding moiety is fused to either one of the first antigen binding moiety or the second antigen binding moiety.

In an aspect, the third antigen binding moiety is a Fab or scFv.

In an aspect, each of the first, second and third antigen binding moiety is a Fab molecule, wherein the third antigen binding moiety is fused at the C-terminus of the Fab heavy chain (CH1) to the N-terminus of the Fab heavy chain of either one of the first antigen binding moiety or the second antigen binding moiety, optionally via a peptide linker.

In an aspect, the peptide linker is selected from the group consisting of the amino acid sequence of SEQ ID NO: 248, SEQ ID NO: 249 or SEQ ID NO: 259.

In an aspect, the first antigen binding moiety is identical to the second antigen binding moiety.

In an aspect, the third antigen binding moiety is a crossover Fab molecule in which the variable regions of the Fab light chain and the Fab heavy chain are exchanged, and wherein each of the first and second antigen binding moiety is a conventional Fab molecule.

In an aspect, in the constant domain CL of the light chain of each of the first and second antigen binding moiety, the amino acid(s) at position 123 and/or 124 is/are substituted independently by lysine (K), arginine (R) or histidine (H) (numbering according to Kabat), and wherein in the constant domain CH1 of the heavy chain of each of the first and second antigen binding moiety, the amino acid at position 147 and/or the amino acid at position 213 is substituted independently by glutamic acid (E) or aspartic acid (D) (numbering according to EU numbering).

In an aspect, the constant domain CL of the light chain of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to EU numbering).

In an aspect, the third antigen-binding moiety capable of binding to DLL3 comprises an antibody variable region comprising any one of (a1) to (a5) below:
(a1) the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239;
(a2) the heavy chain complementarity determining region (CDR) 1 of SEQ ID NO: 276, the heavy chain CDR 2 of SEQ ID NO: 277, the heavy chain CDR 3 of SEQ ID NO: 278, the light chain CDR 1 of SEQ ID NO: 279, the light chain CDR 2 of SEQ ID NO: 280 and the light chain CDR 3 of SEQ ID NO: 281;
(a3) the heavy chain complementarity determining region (CDR) 1 of SEQ ID NO: 285, the heavy chain CDR 2 of SEQ ID NO: 286, the heavy chain CDR 3 of SEQ ID NO: 287, the light chain CDR 1 of SEQ ID NO: 288, the light chain CDR 2 of SEQ ID NO: 289 and the light chain CDR 3 of SEQ ID NO: 290;
(a4) an antibody variable region that binds to the same epitope of any of the antibody variable region selected from (a1) to (a3); and
(a5) an antibody variable fragment that competes with the binding of any of the antibody variable fragment selected from (a1) to (a3).

In an aspect, the third antigen-binding moiety capable of binding to DLL3 comprises an antibody variable region comprising any one of (a1) to (a6) below:
(a1) a heavy chain variable region comprising an amino acid sequence of SEQ ID NO: 232, and a light chain variable region comprising an amino acid sequence of SEQ ID NO: 236;
(a2) a heavy chain variable region comprising an amino acid sequence of SEQ ID NO: 264, and a light chain variable region comprising an amino acid sequence of SEQ ID NO: 265;
(a3) a heavy chain variable region comprising an amino acid sequence of SEQ ID NO: 266, and a light chain variable region comprising an amino acid sequence of SEQ ID NO: 267;
(a4) a heavy chain variable region comprising an amino acid sequence of SEQ ID NO: 268, and a light chain variable region comprising an amino acid sequence of SEQ ID NO: 269;
(a5) an antibody variable region that binds to the same epitope of any of the antibody variable region selected from (a1) to (a4); and
(a6) an antibody variable fragment that competes with the binding of any of the antibody variable fragment selected from (a1) to (a4).

In an aspect, the multispecific antigen-binding molecule of the present invention further comprises a Fc domain.

In an aspect, the Fc domain is composed of a first and a second Fc region subunit capable of stable association, and wherein the Fc domain exhibits reduced binding affinity to human Fc gamma receptor, as compared to a native human IgG1 Fc domain.

In an aspect, the Fc domain exhibits enhanced FcRn-binding activity under an acidic pH condition (e.g., pH 5.8) as compared to that of an Fc region of a native IgG.

In an aspect, the Fc domain comprises Ala at position 434; Glu, Arg, Ser, or Lys at position 438; and Glu, Asp, or Gln at position 440, according to EU numbering.

In an aspect, the Fc domain comprises Ala at position 434; Arg or Lys at position 438; and Glu or Asp at position 440, according to EU numbering.

In an aspect, the Fc domain further comprises Ile or Leu at position 428; and/or Ile, Leu, Val, Thr, or Phe at position 436, according to EU numbering.

In an aspect, the Fc domain comprises a combination of amino acid substitutions selected from the group consisting of:
(a) N434A/Q438R/S440E;
(b) N434A/Q438R/S440D;
(c) N434A/Q438K/S440E;
(d) N434A/Q438K/S440D;
(e) N434A/Y436T/Q438R/S440E;
(f) N434A/Y436T/Q438R/S440D;
(g) N434A/Y436T/Q438K/S440E;
(h) N434A/Y436T/Q438K/S440D;
(i) N434A/Y436V/Q438R/S440E;
(j) N434A/Y436V/Q438R/S440D;
(k) N434A/Y436V/Q438K/S440E;
(1) N434A/Y436V/Q438K/S440D;
(m) N434A/R435H/F436T/Q438R/S440E;
(n) N434A/R435H/F436T/Q438R/S440D;
(o) N434A/R435H/F436T/Q438K/S440E;
(p) N434A/R435H/F436T/Q438K/S440D;
(q) N434A/R435H/F436V/Q438R/S440E;
(r) N434A/R435H/F436V/Q438R/S440D;
(s) N434A/R435H/F436V/Q438K/S440E;
(t) N434A/R435H/F436V/Q438K/S440D;
(u) M428L/N434A/Q438R/S440E;
(v) M428L/N434A/Q438R/S440D;
(w) M428L/N434A/Q438K/S440E;
(x) M428L/N434A/Q438K/S440D;
(y) M428L/N434A/Y436T/Q438R/S440E;
(z) M428L/N434A/Y436T/Q438R/S440D;
(aa) M428L/N434A/Y436T/Q438K/S440E;
(ab) M428L/N434A/Y436T/Q438K/S440D;
(ac) M428L/N434A/Y436V/Q438R/S440E;
(ad) M428L/N434A/Y436V/Q438R/S440D;
(ae) M428L/N434A/Y436V/Q438K/S440E;
(af) M428L/N434A/Y436V/Q438K/S440D;
(ag) L235R/G236R/S239K/M428L/N434A/Y436T/Q438R/S440E; and
(ah) L235R/G236R/A327G/A330S/P331S/M428L/N434A/Y436T/Q438R/S440E, according to EU numbering.

In an aspect, the Fc domain comprises a combination of amino acid substitutions of M428L/N434A/Q438R/S440E.

In an aspect, the Fc domain is an IgG Fc domain, preferably a human IgG Fc domain, more preferably a human IgG1 Fc domain.

In an aspect, the Fc domain comprises any of:
(a) a first Fc subunit comprising an amino acid sequence shown in SEQ ID NO: 100 and a second Fc subunit comprising an amino acid sequence shown in SEQ ID NO: 111; or
(b) a first Fc subunit comprising an amino acid sequence shown in SEQ ID NO: 99 and a second Fc subunit comprising an amino acid sequence shown in SEQ ID NO: 109.

In an aspect, each of the first and second antigen-binding moiety is a Fab, wherein the first antigen-binding moiety is fused at the C-terminus of the Fab heavy chain to the N-terminus of the first or second subunit of the Fc domain, and the second antigen-binding moiety is fused at the C-terminus of the Fab heavy chain to the N-terminus of the remaining subunit of the Fc domain.

In an aspect, the third antigen binding moiety is fused at the C-terminus to the N-terminus of the Fab heavy chain of either one of the first antigen binding moiety or the second antigen binding moiety, optionally via a peptide linker.

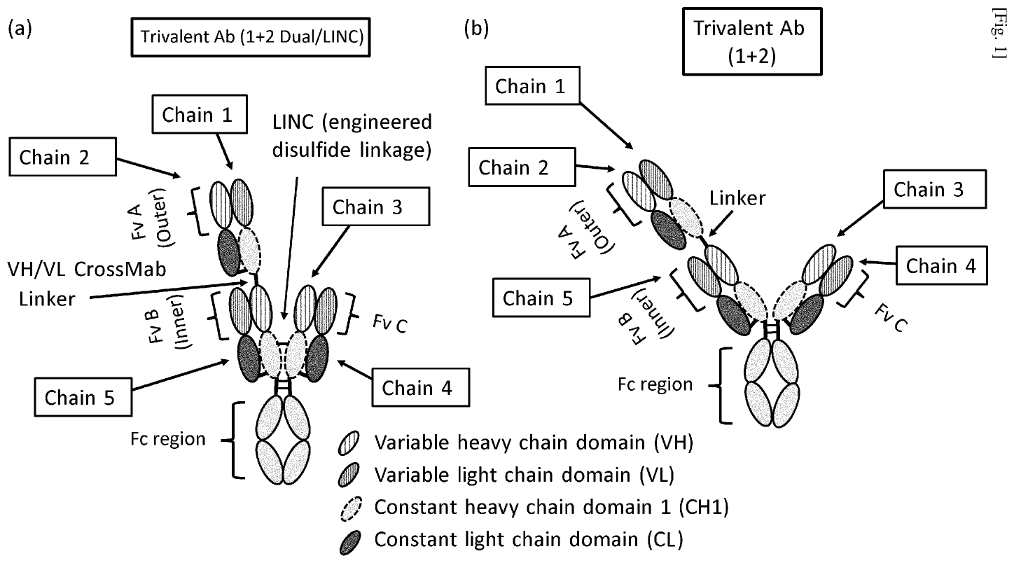

In an aspect, the multispecific antigen-binding molecule of the present invention comprises five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NO: 221 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NO: 223 (chain 1), a polypeptide chain comprising an amino acid sequence of SEQ ID NO: 206 (chain 2), a polypeptide chain comprising an amino acid sequence of SEQ ID NO: 212 (chain 3) and two polypeptide chains each comprising an amino acid sequence of SEQ ID NO: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).

The components of the multispecific antigen binding molecules of the present invention can be fused to each other in a variety of configurations. Exemplary configurations are depicted in FIG. 1(a) read together with Table 2.

According to any of the above embodiments, components of the multispecific antigen binding molecules (e.g. antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G4S)n, (SG4)n, (G4S)n or G4(SG4)n peptide linkers, wherein n is generally a number between 1 and 10, typically between 2 and 4.

Pyroglutamylation

It is known that when an antibody is expressed in cells, the antibody is modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (Journal of Pharmaceutical Sciences, 2008, Vol. 97, p. 2426-2447).

In some embodiments, the multispecific antigen binding molecules of the present invention also includes posttranslational modification. Examples of posttranslational includes undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

Antigen Binding Moiety

As used herein, the term “antigen binding moiety” refers to a polypeptide molecule that specifically binds to an antigen. In one embodiment, an antigen binding moiety is able to direct the entity to which it is attached to a target site, for example to a specific type of tumor cell expressing the cancer antigen (DLL3). In another embodiment an antigen binding moiety is able to activate signaling through its target antigen, for example a T cell receptor complex antigen (in particular CD3) and/or a co-stimulatory receptor (CD137). Antigen binding moieties include antibodies and fragments thereof as further defined herein. Particular antigen binding moieties include an antigen binding domain or an antibody variable region of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: alpha, delta, epsilon, gamma, or mu. Useful light chain constant regions include any of the two isotypes: kappa and lambda.

As used herein, the terms “first”, “second” and “third” with respect to antigen binding moieties etc., are used for convenience of distinguishing when there is more than one of each type of moiety. Use of these terms is not intended to confer a specific order or orientation of the multispecific antigen binding molecule unless explicitly so stated.

Antigen-Binding Moiety Capable of Binding to CD3 and CD137 but not at the Same Time

The multispecific antigen binding molecule described herein comprises at least one antigen binding moiety capable of binding to CD3 and CD137, but does not bind to CD3 and CD137 at the same time (also referred to herein as “Dual antigen binding moiety” or “first antigen binding moiety” or “Dual-Fab” or “Dual-Ig”). In a particular embodiment, the multispecific antigen binding molecule comprises two Dual antigen binding moiety (“first antigen binding moiety” or “second antigen binding moiety” or “Dual-Fab”). In some embodiments, each of the two Dual antigen binding moiety (“first antigen binding moiety” or “second antigen binding moiety” or “Dual-Fab”) provides monovalent binding to CD3 or CD137, but does not bind to CD3 and CD137 at the same time. In a particular embodiment, the multispecific antigen binding molecule comprises not more than two the Dual antigen binding moiety (“first antigen binding moiety” or “second antigen binding moiety” or “Dual-Fab”).

In certain embodiments, the Dual antigen binding moiety (“first antigen binding moiety” or “second antigen binding moiety” or “Dual-Fab”) is generally a Fab molecule, particularly a conventional Fab molecule. In certain embodiments, the Dual antigen binding moiety (“first antigen binding moiety” or “second antigen binding moiety” is a domain comprising antibody light-chain and heavy-chain variable regions (VL and VH). Suitable examples of such domains comprising antibody light-chain and heavy-chain variable regions include “single chain Fv (scFv)”, “single chain antibody”, “Fv”, “single chain Fv 2 (scFv2)”, “Fab”, “F(ab′)2”, etc.

In certain embodiments, the Dual antigen binding moiety (“first antigen binding moiety” or “second antigen binding moiety” or “Dual-Fab”) specifically binds to the whole or a portion of a partial peptide of CD3. In a particular embodiment CD3 is human CD3 or cynomolgus CD3, most particularly human CD3. In a particular embodiment the first antigen binding moiety is cross-reactive for (i.e. specifically binds to) human and cynomolgus CD3. In some embodiments, the first antigen binding moiety is capable of specific binding to the epsilon subunit of CD3, in particular the human CD3 epsilon subunit of CD3 which is shown in SEQ ID NOs: 7 (NP_000724.1) (RefSeq registration numbers are shown within the parentheses). In some embodiments, the Dual antigen binding moiety (“first antigen binding moiety” or “second antigen binding moiety” or “Dual-Fab”) is capable of specific binding to the CD3 epsilon chain expressed on the surface of eukaryotic cells. In some embodiments, the first antigen binding moiety binds to the CD3 epsilon chain expressed on the surface of T cells.

In certain embodiments, the CD137 is human CD137. In some embodiments, favorable examples of an antigen-binding molecule of the present invention comprises Dual antigen binding moiety (“first antigen binding moiety” or “second antigen binding moiety” or “Dual-Fab”) that bind to the same epitope as the human CD137 epitope bound by the antibody selected from the group consisting of:
antibody that recognize a region comprising the SPCPPNSFSSAGGQRTCD ICRQCKGVFRTRKECSSTSNAECDCTPGFHCLGAGCSMCEQDCKQGQELTK KGC
sequence (SEQ ID NO: 21),
antibody that recognize a region comprising the DCTPGFHCLGAGCSMCEQDC KQGQELTKKGC sequence (SEQ ID NO: 35),
antibody that recognize a region comprising the LQDPCSNC PAGTFCDNNRNQICSPCPPNSFSSAGGQRTCDICRQCKGVFRTRKECSSTSNA EC
sequence (SEQ ID NO: 49), and
antibody that recognize a region comprising the LQDPCSNCPAGTFCDNNRN QIC sequence (SEQ ID NO: 105) in the human CD137 protein.

In specific embodiments, the Dual antigen binding moiety (“first antigen binding moiety” or “second antigen binding moiety” or “Dual-Fab”) comprises any one of the antibody variable region sequences shown in Tables 1A below. In specific embodiments, the Dual antigen binding moiety (“first antigen binding moiety” or “second antigen binding moiety” or “Dual-Fab”) comprises any one of the combinations of the heavy chain variable region and light chain variable region shown in Table 1A.

TABLE 1A
SEQ ID NOs of the variable regions of the Dual antigen
binding moiety (“first antigen binding moiety”
or “second antigen binding moiety” or “Dual-Fab”)

SEQ ID NOs

| | Heavy chain | Light chain |
|---|---|---|
| Name | variable region (VH) | variable region (VL) |
| DualAE08 | 3 | 59 |
| DualAE06 | 4 | 58 |
| DualAE17 | 5 | 58 |
| DualAE10 | 5 | 60 |
| DualAE05 | 6 | 58 |
| DualAE19 | 8 | 58 |
| DualAE20 | 9 | 58 |
| DualAE21 | 9 | 61 |
| DualAE22 | 10 | 58 |
| DualAE23 | 11 | 61 |
| DualAE09 | 12 | 61 |
| DualAE18 | 12 | 58 |
| DualAE14 | 13 | 58 |
| DualAE15 | 14 | 58 |
| DualAE16 | 81 | 60 |

In one embodiment the Dual antigen binding moiety (“first antigen binding moiety” or “second antigen binding moiety” or “Dual-Fab”) comprises a heavy chain variable region sequence that is at least about 95%, 96%, 97%, 98%, 99% or 100% identical to SEQ ID NO: 6 and a light chain variable region sequence that is at least about 95%, 96%, 97%, 98%, 99% or 100% identical to SEQ ID NO: 58. In one embodiment the Dual antigen binding moiety (“first antigen binding moiety” or “second antigen binding moiety” or “Dual-Fab”) comprises a heavy chain variable region comprising the amino acid sequence of SEQ ID NO: 6 and a light chain variable region comprising the amino acid sequence of SEQ ID NO: 58.

In one embodiment the Dual antigen binding moiety (“first antigen binding moiety” or “second antigen binding moiety” or “Dual-Fab”) comprises a heavy chain variable region sequence that is at least about 95%, 96%, 97%, 98%, 99% or 100% identical to SEQ ID NO: 14 and a light chain variable region sequence that is at least about 95%, 96%, 97%, 98%, 99% or 100% identical to SEQ ID NO: 58. In one embodiment the Dual antigen binding moiety (“first antigen binding moiety” or “second antigen binding moiety” or “Dual-Fab”) comprises a heavy chain variable region comprising the amino acid sequence of SEQ ID NO: 14 and a light chain variable region comprising the amino acid sequence of SEQ ID NO: 58.

In one embodiment the Dual antigen binding moiety (“first antigen binding moiety” or “second antigen binding moiety” or “Dual-Fab”) comprises a heavy chain variable region sequence that is at least about 95%, 96%, 97%, 98%, 99% or 100% identical to SEQ ID NO: 81 and a light chain variable region sequence that is at least about 95%, 96%, 97%, 98%, 99% or 100% identical to SEQ ID NO: 58. In one embodiment the Dual antigen binding moiety (“first antigen binding moiety” or “second antigen binding moiety” or “Dual-Fab”) comprises a heavy chain variable region comprising the amino acid sequence of SEQ ID NO: 81 and a light chain variable region comprising the amino acid sequence of SEQ ID NO: 58.

In specific embodiments, the Dual antigen binding moiety (“first antigen binding moiety” or “second antigen binding moiety” or “Dual-Fab”) comprises any one of the combinations of HVR sequences shown in Table 1B below.

TABLE 1B
SEQ ID NOs of the HVR (CDR) sequences of the Dual antigen
binding moiety (“first antigen binding moiety”
or “second antigen binding moiety” or “Dual-Fab”)

| | SEQ ID NOs | | | | | |
|---|---|---|---|---|---|---|
| | HVR- | HVR- | HVR- | HVR- | HVR- | HVR- |
| Name | H1 | H2 | H3 | L1 | L2 | L3 |
| DualAE08 | 17 | 31 | 45 | 64 | 69 | 74 |
| DualAE06 | 18 | 32 | 46 | 63 | 68 | 73 |
| DualAE17 | 19 | 33 | 47 | 63 | 68 | 73 |
| DualAE10 | 19 | 33 | 47 | 65 | 70 | 75 |
| DualAE05 | 20 | 34 | 48 | 63 | 68 | 73 |
| DualAE19 | 22 | 36 | 50 | 63 | 68 | 73 |
| DualAE20 | 23 | 37 | 51 | 63 | 68 | 73 |
| DualAE21 | 23 | 37 | 51 | 66 | 71 | 76 |
| DualAE22 | 24 | 38 | 52 | 63 | 68 | 73 |
| DualAE23 | 25 | 39 | 53 | 66 | 71 | 76 |
| DualAE09 | 26 | 40 | 54 | 66 | 71 | 76 |
| DualAE18 | 26 | 40 | 54 | 63 | 68 | 73 |
| DualAE14 | 27 | 41 | 55 | 63 | 68 | 73 |
| DualAE15 | 28 | 42 | 56 | 63 | 68 | 73 |
| DualAE16 | 82 | 83 | 84 | 65 | 70 | 75 |

In some embodiments, the Dual antigen binding moiety (“first antigen binding moiety” or “second antigen binding moiety” or “Dual-Fab”) each comprises antibody variable region comprising the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

In some embodiments, the multispecific antigen binding molecules or the Dual antigen binding moiety (“first antigen binding moiety” or “second antigen binding moiety” or “Dual-Fab”) of the present invention also includes posttranslational modification. Examples of posttranslational includes undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

Antigen-Binding Moiety Capable of Binding to DLL3

The multispecific antigen binding molecule described herein comprises at least one antigen binding moiety capable of binding to Delta-like 3 (DLL3) (also referred to herein as a “DLL3 antigen binding moiety” or “third antigen binding moiety”).

In certain embodiments, the multispecific antigen binding molecule comprises one antigen binding moiety capable of binding to DLL3. In certain embodiments, the multispecific antigen binding molecule comprises two antigen binding moieties capable of binding to DLL3 (“DLL3 antigen binding moiety”). In a particular such embodiment, each of these antigen binding moieties specifically binds to the same epitope of DLL3. In an even more particular embodiment, all of these “DLL3 antigen binding moiety” are identical. In one embodiment, the multispecific antigen binding molecule comprises an immunoglobulin molecule capable of specific binding to DLL3 (“DLL3 antigen binding moiety”). In one embodiment the multispecific antigen binding molecule comprises not more than two antigen binding moieties capable of binding to DLL3 (“DLL3 antigen binding moiety”).

In certain embodiments, the DLL3 antigen binding moiety is a crossover Fab molecule, i.e. a DLL3 molecule wherein either the variable or the constant regions of the Fab heavy and light chains are exchanged. In certain embodiments, the DLL3 antigen binding moiety is a crossover Fab molecule in which the variable regions of the Fab light chain and the Fab heavy chain are exchanged.

In some embodiments, the DLL3 antigen binding moiety binds specifically to the extracellular domain of DLL3. In some embodiments, the DLL3 antigen binding moiety binds specifically to an epitope within the extracellular domain of DLL3. In some embodiments, the DLL3 antigen binding moiety binds to the DLL3 protein expressed on the surface of eukaryotic cells. In some embodiments, the DLL3 antigen binding moiety binds to the DLL3 protein expressed on the surface of cancer cells.

In some embodiments, the multispecific antigen-binding molecules or the DLL3 antigen binding moiety bind to an epitope within the extracellular domain (ECD), i.e., the domain from the N-terminus to immediately before the TM region, but not to the TM region or the C-terminal intracellular domain. The multispecific antigen-binding molecules or the DLL3 antigen binding moiety may bind to an epitope within any of the above-mentioned domains/regions within the ECD. In preferred embodiments, the multispecific antigen-binding molecules or the DLL3 antigen binding moiety bind to an epitope within the region from EGF6 to immediately before the TM region. More specifically, the multispecific antigen-binding molecules or the DLL3 antigen binding moiety may bind to an epitope within the regions defined in SEQ ID NO: 89 in human DLL3. In some embodiments, the multispecific antigen-binding molecules or the DLL3 antigen binding moiety bind to the EGF1, EGF2, EGF3, EGF4, EGF5, or EGF6 region or a region from EGF6 to immediately before the TM region of human DLL3, or an epitope within the EGF1, EGF2, EGF3, EGF4, EGF5, or EGF6 region or a region from EGF6 to immediately before the TM region of human DLL3. In some embodiments, the multispecific antigen-binding molecules or the DLL3 antigen binding moiety can be derived from previously reported anti-DLL3 antibodies in which the DLL3 epitopes bound have been characterized (e.g. WO2019131988 and WO2011093097).

In specific embodiments, the multispecific antigen-binding molecules or the DLL3 antigen binding moiety comprises any one of the antibody variable region sequences shown in Tables 1C below. In specific embodiments, the multispecific antigen-binding molecules or the DLL3 antigen binding moiety comprises any one of the combinations of the heavy chain variable region and light chain variable region shown in Table 1C. In some embodiments, multispecific antigen-binding molecules or the DLL3 antigen binding moiety comprises is a domain that comprises an antibody variable fragment that competes for binding to DLL3 with any one of the antibody variable regions shown in Table 1C.

TABLE 1C
SEQ ID NOs of the variable regions of the
exemplary DLL3 antigen binding moiety

SEQ ID NOs

| | Heavy chain | Light chain |
|---|---|---|
| Name | variable region (VH) | variable region (VL) |
| DL301 | 305 | 313 |
| DL306 | 306 | 314 |
| DL309 | 307 | 315 |
| DL312 | 308 | 316 |
| DLL3-14 | 309 | 317 |
| DLL3-22 | 310 | 318 |
| DLL3-4 | 311 | 319 |
| DLL3-6 | 312 | 320 |
| DLA0106 | 260 | 261 |
| DLA0126 | 262 | 263 |
| DLA0316 | 264 | 265 |
| DLA0379 | 266 | 267 |
| DLA0580 | 268 | 269 |
| DLA0641 | 270 | 271 |
| DLA0769 | 272 | 273 |
| DLA0841 | 274 | 275 |
| D30841AE05 | 297 | 236 |
| D30841AE08 | 298 | 236 |
| D30841AE11 | 298 | 302 |
| D30841AE12 | 299 | 236 |
| D30841AE13 | 232 | 236 |
| D30841AE14 | 300 | 236 |
| D30841AE15 | 301 | 236 |

In one embodiment the DLL3 antigen binding moiety comprises a heavy chain variable region sequence that is at least about 95%, 96%, 97%, 98%, 99% or 100% identical to SEQ ID NO: 232 and a light chain variable region sequence that is at least about 95%, 96%, 97%, 98%, 99% or 100% identical to SEQ ID NO: 236. In one embodiment the DLL3 antigen binding moiety comprises a heavy chain variable region comprising the amino acid sequence of SEQ ID NO: 232 and a light chain variable region comprising the amino acid sequence of SEQ ID NO: 236.

In one embodiment the DLL3 antigen binding moiety comprises a heavy chain variable region sequence that is at least about 95%, 96%, 97%, 98%, 99% or 100% identical to SEQ ID NO: 300 and a light chain variable region sequence that is at least about 95%, 96%, 97%, 98%, 99% or 100% identical to SEQ ID NO: 236. In one embodiment the DLL3 antigen binding moiety comprises a heavy chain variable region comprising the amino acid sequence of SEQ ID NO: 300 and a light chain variable region comprising the amino acid sequence of SEQ ID NO: 236.

In one embodiment the DLL3 antigen binding moiety comprises a heavy chain variable region sequence that is at least about 95%, 96%, 97%, 98%, 99% or 100% identical to SEQ ID NO: 301 and a light chain variable region sequence that is at least about 95%, 96%, 97%, 98%, 99% or 100% identical to SEQ ID NO: 236. In one embodiment the DLL3 antigen binding moiety comprises a heavy chain variable region comprising the amino acid sequence of SEQ ID NO: 301 and a light chain variable region comprising the amino acid sequence of SEQ ID NO: 236.

In one embodiment the DLL3 antigen binding moiety comprises a heavy chain variable region sequence that is at least about 95%, 96%, 97%, 98%, 99% or 100% identical to SEQ ID NO: 274 and a light chain variable region sequence that is at least about 95%, 96%, 97%, 98%, 99% or 100% identical to SEQ ID NO: 275. In one embodiment the DLL3 antigen binding moiety comprises a heavy chain variable region comprising the amino acid sequence of SEQ ID NO: 274 and a light chain variable region comprising the amino acid sequence of SEQ ID NO: 275.

In one embodiment the DLL3 antigen binding moiety comprises a heavy chain variable region sequence that is at least about 95%, 96%, 97%, 98%, 99% or 100% identical to SEQ ID NO: 264 and a light chain variable region sequence that is at least about 95%, 96%, 97%, 98%, 99% or 100% identical to SEQ ID NO: 265. In one embodiment the DLL3 antigen binding moiety comprises a heavy chain variable region comprising the amino acid sequence of SEQ ID NO: 264 and a light chain variable region comprising the amino acid sequence of SEQ ID NO: 265.

In specific embodiments, the DLL3 antigen binding moiety comprises any one of the combinations of HVR sequences shown in Table 1D below. In some embodiments, multispecific antigen-binding molecules or the DLL3 antigen binding moiety comprises is a domain that comprises an antibody variable fragment that competes for binding to DLL3 with any one of the antibody variable regions shown in Table 1D, or competes for binding to DLL3 with any antibody variable fragment that comprises the HVR sequence identical with the HVR regions of the antibody variable regions shown in Table 1D.

TABLE 1D
SEQ ID NOs of the HVR (CDR) sequences of
exemplary DLL3 antigen binding moiety

| | SEQ ID NOs | | | | | |
|---|---|---|---|---|---|---|
| | HVR- | HVR- | HVR- | HVR- | HVR- | HVR- |
| Name | H1 | H2 | H3 | L1 | L2 | L3 |
| DLA0316 | 276 | 277 | 278 | 279 | 280 | 281 |
| DLA0580 | 285 | 286 | 287 | 288 | 289 | 290 |
| DLA0769 | 291 | 292 | 293 | 294 | 295 | 296 |
| DLA0841 | 282 | 283 | 284 | 237 | 238 | 239 |
| D30841AE05 | 233 | 234 | 303 | 237 | 238 | 239 |
| D30841AE08 | 233 | 234 | 235 | 237 | 238 | 239 |
| D30841AE11 | 233 | 234 | 235 | 237 | 238 | 304 |
| D30841AE12 | 233 | 234 | 235 | 237 | 238 | 239 |
| D30841AE13 | 233 | 234 | 235 | 237 | 238 | 239 |
| D30841AE14 | 233 | 234 | 235 | 237 | 238 | 239 |
| D30841AE15 | 233 | 234 | 235 | 237 | 238 | 239 |

In some embodiments, the multispecific antigen binding molecules or the DLL3 antigen binding moiety of the present invention comprises an antibody variable region comprising any one of (a1) to (a5) below:
(a1) the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239;
(a2) the heavy chain complementarity determining region (CDR) 1 of SEQ ID NO: 276, the heavy chain CDR 2 of SEQ ID NO: 277, the heavy chain CDR 3 of SEQ ID NO: 278, the light chain CDR 1 of SEQ ID NO: 279, the light chain CDR 2 of SEQ ID NO: 280 and the light chain CDR 3 of SEQ ID NO: 281;
(a3) the heavy chain complementarity determining region (CDR) 1 of SEQ ID NO: 285, the heavy chain CDR 2 of SEQ ID NO: 286, the heavy chain CDR 3 of SEQ ID NO: 287, the light chain CDR 1 of SEQ ID NO: 288, the light chain CDR 2 of SEQ ID NO: 289 and the light chain CDR 3 of SEQ ID NO: 290;
(a4) an antibody variable region that binds to the same epitope of any of the antibody variable region selected from (a1) to (a3); and
(a5) an antibody variable fragment that competes with the binding of any of the antibody variable fragment selected from (a1) to (a3).

In some embodiments, the multispecific antigen binding molecules or the DLL3 antigen binding moiety of the present invention also includes posttranslational modification. Examples of posttranslational includes undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

In another aspect, the DLL3 antigen binding moiety of the present invention can be used in novel chimeric antigen receptor (CAR) incorporating a DLL3 binding domain (DLL3 CAR). In certain embodiments, a DLL3 binding domain (and DLL3 CAR) of the invention will comprise a scFv construct, and in a preferred embodiment, will comprise and comprise a heavy and light chain variable region as disclosed herein. In other preferred embodiments, the DLL3 binding domain (and DLL3 CAR) of the invention will comprise a scFv construct or fragment thereof comprising the heavy and light chain variable regions disclosed herein. In a preferred embodiment, the disclosed chimeric antigen receptors are useful for treating or preventing a proliferative disorder and any recurrence or metastasis thereof.

In certain embodiments, the DLL3 protein is expressed on tumor-initiating cells. DLL3 CAR is expressed on cytotoxic lymphocytes (preferably autologous cytotoxic lymphocytes) via genetic modification (eg, transduction), resulting in DLL3-sensitive lymphocytes that can be used to target and kill DLL3-positive tumor cells. As will be broadly discussed herein, CARs of the invention typically comprise an extracellular domain, a transmembrane domain, and an intracellular signaling domain comprising a DLL3 binding domain that activates certain lymphocytes and produces immune response of DLL3 positive tumor cells. Selected embodiments of the invention comprise immunologically active host cells which exhibit the disclosed CAR and various polynucleotide sequences and vectors encoding the DLL3 CAR of the invention. Other aspects include methods of enhancing the activity of T lymphocytes or natural killer (NK) cells in an individual by introducing a host cell expressing a DLL3 CAR molecule into an individual suffering from cancer and treating the individual. Such aspects include, inter alia, lung cancer (eg, small cell lung cancer) and melanoma.

Methods for Producing Multispecific Antigen-Binding Molecules

The present disclosure provides methods of producing any multispecific antigen-binding molecules described herein.

In an aspect, the present disclosure provides a method for producing a multispecific antigen-binding molecule, wherein the multispecific antigen-binding molecule comprises:
a first antigen-binding moiety and a second antigen-binding moiety; wherein each of the first antigen-binding moiety and the second antigen-binding is a Fab and is capable of binding to a first antigen and a second antigen different from the first antigen, but does not bind both antigens at the same time; and
a third antigen-binding moiety comprising a heavy chain variable region (VH) and a light chain variable region (VL), which is capable of binding to a third antigen different from the first and the second antigen, preferably an antigen expressed on a cancer cell/tissue,
the method comprising:
(a) providing one or more nucleic acid(s) encoding:
i. a first polypeptide comprising (starting from N-terminus to C-terminus) the VH or VL of the third antigen-binding moiety, optionally a heavy chain constant region (CH1); and the VH or VL of the first antigen-binding moiety, a heavy chain constant region (CH1); and optionally a hinge region and/or a Fc region (CH2 and CH3);
ii. a second polypeptide comprising (starting from N-terminus to C-terminus) the VH or VL of the third antigen-binding moiety, optionally a light chain constant region (CL);
iii. a third polypeptide comprising (starting from N-terminus to C-terminus) a VH or VL of the second antigen-binding moiety, a heavy chain constant region (CH1); and optionally a hinge region and/or a Fc region (CH2 and CH3);
iv. a fourth polypeptide comprising (starting from N-terminus to C-terminus) a VH or VL of the second antigen-binding moiety, optionally a light chain constant region (CL); and
v. a fifth polypeptide comprising (starting from N-terminus to C-terminus) a VH or VL of the first antigen-binding moiety, optionally a light chain constant region (CL)
(b) introducing the one or more nucleic acid(s) produced in (a) into a host cell;
(c) culturing the host cell such that the polypeptides in (i) to (v) are expressed; and
(d) collecting the multispecific antigen-binding molecule comprising the five polypeptides in (i) to (v) from the culture solution of the cell cultured in step (c); and
wherein optionally the polypeptides in (iv) to (v) are identical; and
wherein each of the first antigen-binding moiety and the second antigen-binding moiety comprises at least one cysteine residue (via mutation, substitution or insertion) which is not in a hinge region, preferably the at least one cysteine locates in the CH1 region; the at least one cysteine residue is capable of forming at least one disulfide bond between the first antigen-binding moiety and the second antigen-binding moiety, preferably in the CH1 region;
wherein the method comprises contacting the preparation with a reducing reagent.

In an aspect, each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.

In an aspect, the method further comprises step (e) contacting the multispecific antigen-binding molecule (multispecific antigen binding molecule) preparation collected from step (d) with a reducing reagent under reducing conditions which allow the cysteine(s) in the CH1 region (position 191 according to EU numbering) to form one or more disulfide bond.

In an aspect, the multispecific antigen binding molecule preparation collected from step (d) (before contacting with the reducing agent) comprises two or more structural isoforms which differ by at least one disulfide bond formed between amino acid residues located in the CH1 region or at the position 191 in the CH1 region (EU numbering), and the step (e) contacting with reducing agent preferentially enriches or increases the population of a multispecific antigen binding molecule structural isoform having at least one disulfide bond formed between amino acid residues located in the CH1 region or at the position 191 in the CH1 region (EU numbering).

In an aspect, the pH of the reducing reagent contacting with the multispecific antigen binding molecule is from about 3 to about 10.

In an aspect, the pH of the reducing reagent contacting with the multispecific antigen binding molecule is about 6, 7 or 8.

In an aspect, the pH of the reducing reagent contacting with the multispecific antigen binding molecule is about 7.

In an aspect, the pH of the reducing reagent contacting with the multispecific antigen binding molecule is about 3.

In an aspect, the reducing agent is selected from the group consisting of TCEP, 2-MEA, DTT, Cysteine, GSH and Na₂SO₃.

In an aspect, the reducing agent is TCEP, preferably 0.25 mM TCEP.

In an aspect, the concentration of the reducing agent is from about 0.01 mM to about 100 mM.

In an aspect, the concentration of the reducing agent is about 0.01, 0.05, 0.1, 0.25, 0.5, 1, 2.5, 5, 10, 25, 50, 100 mM, preferably about 0.25 mM.

In an aspect, the contacting step is performed for at least 30 minutes.

In an aspect, the contacting step is performed for about 10 minutes to about 48 hours.

In an aspect, the contacting step is performed for about 2 hours or about 18 hours.

In an aspect, the contacting step is performed at a temperature of about 4 degrees Celsius to 37 degrees Celsius, preferably at 23 degrees Celsius to 25 degrees Celsius.

In an aspect, the multispecific antigen binding molecule is at least partially purified prior to the contacting step with reducing agent.

In an aspect, the multispecific antigen binding molecule is partially purified by affinity chromatography (preferably Protein A chromatography) prior to the contacting.

In an aspect, the concentration of the multispecific antigen binding molecule is from about 0.1 mg/ml to about 50 mg/ml or more.

In an aspect, the concentration of the multispecific antigen binding molecule is about 10 mg/ml or about 20 mg/ml.

In an aspect, the method further comprises a step of promoting re-oxidization of cysteine disulfide bonds, preferably by removing the reducing agent, preferably by dialysis or buffer exchange.

In an aspect, the third antigen-binding moiety is a conventional Fab, and wherein
(a) the first polypeptide comprising (starting from N-terminus to C-terminus) the VH of the third antigen-binding moiety, a heavy chain constant region (CH1); and the VH of the first antigen-binding moiety, a heavy chain constant region (CH1); and optionally a hinge region and/or a Fc region (CH2 and CH3);
(b) a second polypeptide comprising (starting from N-terminus to C-terminus) the VL of the third antigen-binding moiety, and a light chain constant region (CL);
(c) a third polypeptide comprising (starting from N-terminus to C-terminus) a VH of the second antigen-binding moiety, a heavy chain constant region (CH1); and optionally a hinge region and/or a Fc region (CH2 and CH3);
(d) a fourth polypeptide comprising (starting from N-terminus to C-terminus) a VL of the second antigen-binding moiety, and a light chain constant region (CL); and
(e) a fifth polypeptide comprising (starting from N-terminus to C-terminus) a VL of the first antigen-binding moiety, and a light chain constant region (CL).

In an aspect, the third antigen-binding moiety is a VH/VL crossover Fab (in which the variable regions of the Fab light chain and the Fab heavy chain are exchanged), and wherein
(a) the first polypeptide comprising (starting from N-terminus to C-terminus) the VL of the third antigen-binding moiety, a heavy chain constant region (CH1); and the VH of the first antigen-binding moiety, a heavy chain constant region (CH1); and optionally a hinge region and/or a Fc region (CH2 and CH3);
(b) a second polypeptide comprising (starting from N-terminus to C-terminus) the VH of the third antigen-binding moiety, and a light chain constant region (CL);
(c) a third polypeptide comprising (starting from N-terminus to C-terminus) a VH of the second antigen-binding moiety, a heavy chain constant region (CH1); and optionally a hinge region and/or a Fc region (CH2 and CH3);
(d) a fourth polypeptide comprising (starting from N-terminus to C-terminus) a VL of the second antigen-binding moiety, and a light chain constant region (CL); and
(e) a fifth polypeptide comprising (starting from N-terminus to C-terminus) a VL of the first antigen-binding moiety, and a light chain constant region (CL).

In an aspect, in the CL of each of the first and second antigen binding moiety, the amino acids at position 123 and 124 are arginine (R) and lysine (K) respectively (numbering according to Kabat), and wherein in the constant domain CH1 of the heavy chain of each of the first and second antigen binding moiety the amino acids at position 147 and 213 are glutamic acid (E) (numbering according to EU numbering).

In an aspect, wherein in step (a)(i), the first polypeptide, between the third antigen-binding moiety and the VH or VL of the first antigen-binding moiety, further comprises a peptide linker.

In an aspect, the peptide linker is selected from the group consisting of the amino acid sequence of SEQ ID NO: 248, SEQ ID NO: 249 or SEQ ID NO: 259.

In an aspect, each of the first antigen-binding moiety and the second antigen-binding moiety is capable of binding to CD3 and CD137 but does not bind both CD3 and CD137 at the same time.

In an aspect, the first antigen-binding moiety and the second antigen-binding moiety each comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

In an aspect, the first antigen-binding moiety and the second antigen-binding moiety each comprises an antibody variable region comprising any one of (a1) to (a17) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58; and
(a15) a heavy chain variable region comprising an amino acid sequence of SEQ ID NO: 81, and a light chain variable region comprising an amino acid sequence of SEQ ID NO: 60.
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

In an aspect, the third antigen-binding moiety is capable of binding to DLL3, preferably human DLL3.

In an aspect, the third antigen-binding moiety capable of binding to DLL3 comprises an antibody variable region comprising the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239.

In an aspect, the third antigen-binding moiety capable of binding to DLL3 comprises an antibody variable region comprising: a heavy chain variable region comprising an amino acid sequence of SEQ ID NO: 232, and a light chain variable region comprising an amino acid sequence of SEQ ID NO: 236.

In an aspect, the multispecific antigen binding molecule further comprises a Fc domain.

In an aspect, the Fc domain is composed of a first and a second Fc region subunit capable of stable association, and wherein the Fc domain exhibits reduced binding affinity to human Fc gamma receptor, as compared to a native human IgG1 Fc domain.

In an aspect, the multispecific antigen binding molecule comprises five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NO: 221 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NO: 223 (chain 1), a polypeptide chain comprising an amino acid sequence of SEQ ID NO: 206 (chain 2), a polypeptide chain comprising an amino acid sequence of SEQ ID NO: 212 (chain 3) and two polypeptide chains each comprising an amino acid sequence of SEQ ID NO: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).

In an aspect, the fourth polypeptide (chain 4) and the fifth polypeptide (chain 5) are identical.

In an aspect, wherein only one nucleic acid, or two, three, four or five different nucleic acids encode and express the first, second, third, fourth and fifth polypeptides.

Contacting with a Reducing Reagent

By “contacting” is meant subjecting to, exposing to, in solution. The antibody, protein or polypeptide can be contacted with the reducing reagents while also bound to a solid support (e.g., an affinity column or a chromatography matrix). Preferably, the solution is buffered. In order to maximize the yield of antibody/protein with a desired conformation, the pH of the solution is chosen to protect the stability of the antibody/protein and to be optimal for disulfide exchange. In the practice of the invention, the pH of the solution is preferably not strongly acidic. Thus, some pH ranges are greater than pH 5, preferably about pH 6 to about pH 11, more preferably from about pH 7 to about pH 10, and still more preferably from about pH 6 to about pH 8. In one nonlimiting embodiment of the invention, the optimal pH was found to be about pH 7. However, the optimal pH for a particular embodiment of the invention can be easily determined experimentally by those skilled in the art.

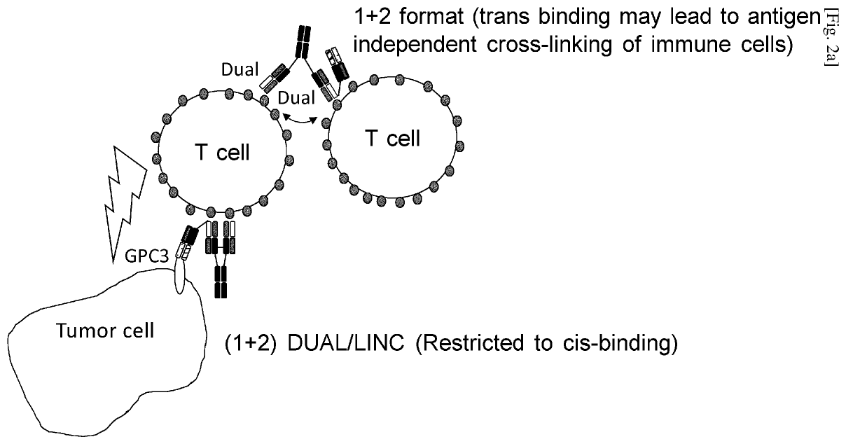

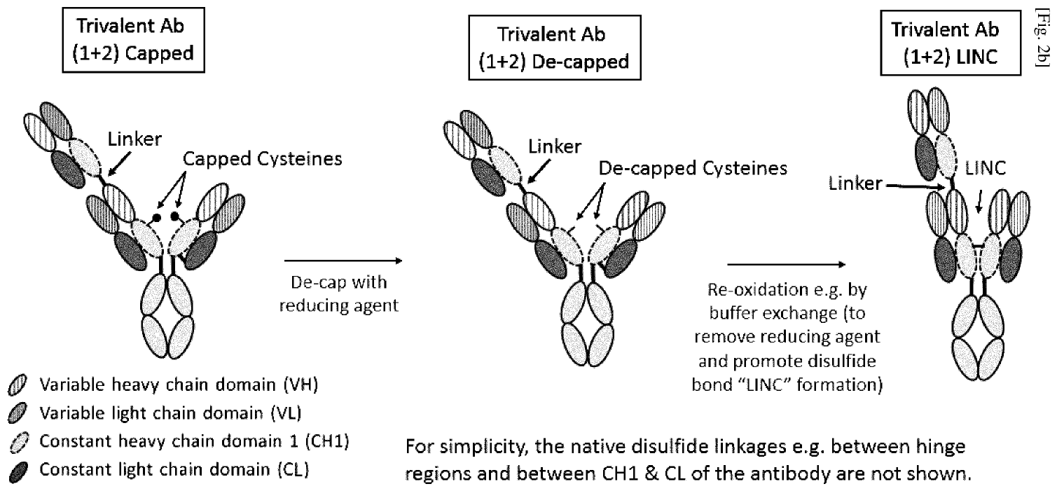

While not wishing to be bound by the following theory, it is believed that the presence of UnLINC format (i.e., trivalent 1+2 antibody without the engineered disulfide bond or “paired cysteines”) could be due to the unpaired Cys residues often form disulfide bonds with molecule that contains free thiol group, such as cysteinylation and glutathionylation which “capped” the unpaired cys residues and prevents LINC formation (formation of engineered disulfide bond). As shown in FIG. 2(b), to remove the capped molecules of unpair cysteines, reducing agents can help de-cap the surface cysteines and further re-oxidation (e.g. remove reducing reagent via buffer exchange) of de-capped antibody can promote disulfide bond formation between the de-capped cysteines for LINC formation. Hence, removal of cysteinylation from the unpaired sulfhydryl in the UnLINC format via reduction and re-oxidation could remove the UnLINC format and improves homogeneity of the antibodies.

The term “reduction reagent” and “reducing agent” is used interchangeably. In some embodiments, said reducing agents are free thiols. The reducing reagent is preferably comprised of a compound from the group consisting of glutathione (GSH), dithiothreitol (DTT), 2-mercaptoethanol, 2-aminoethanethiol (2-MEA), TCEP (tris(2-carboxyethyl)phosphine), dithionitrobenzoate, cysteine and Na₂SO₃. In some embodiments, TCEP, 2-MEA, DTT, cysteine, GSH or Na₂SO₃ can be used. In some preferred embodiments, 2-MEA can be used. In some preferred embodiments, TCEP can be used.

The reducing agent may be added to the fermentation media in which the cells producing the recombinant protein are grown. In additional embodiments, the reducing agent also may be added to the LC mobile phase during the LC separation step for separating the recombinant protein. In certain embodiments, the protein is immobilized to a stationary phase of the LC column and the reducing agents are part of the mobile phase. In specific embodiments, the untreated IgG antibody may elute as a heterogeneous mixture as indicated by the number of peaks. The use of the reduction/oxidation coupling reagent produces a simpler and more uniform peak pattern. It is contemplated that this more uniform peak of interest may be isolated as a more homogeneous preparation of the IgG.

The reducing agent is present at a concentration that is sufficient to increase the relative proportion of the desired conformation (e.g., the “paired cysteines” form of an antibody which has one or more engineered disulfide bond(s) formed between the two Fabs of the antibody, e.g., between amino acid residues which are not in the hinge region). The optimal absolute concentration and molar ratio of the reducing agent depends upon the concentration of total IgG and in some circumstances the specific IgG subclass. When used for preparing IgG1 molecules it also will depend on the number and accessibility of the unpaired cysteines in the protein. Generally, the concentration of free thiols from the reducing agent can be from about 0.05 mM to about 100 mM, more preferably about 0.1 mM to about 50 mM, and still more preferably about 0.2 mM to about 20 mM. In some preferred embodiments, the concentration of the reducing agent is 0.01, 0.05, 0.1, 0.25, 0.5, 1, 2.5, 5, 10, 25, 50, 100 mM. In some preferred embodiments, 0.05 mM to 1 mM of 2-MEA can be used. In some preferred embodiments, 0.01 mM to 25 mM TCEP can be used.

Contacting the preparation of recombinant protein with a reducing agent is performed for a time sufficient to increase the relative proportion of the desired conformation. Any relative increase in proportion is desirable, including for, example, at least 10%, 20%, 30%, 40%, 50%, 60%, 70% and even 80% or 90% of the protein with an undesired conformation is converted to protein with the desired conformation. The contacting may be performed by providing the reducing agent to the fermentation medium in which the protein is being generated. Alternatively, the contacting takes place upon partial purification of the protein from the cell culture in which it is generated. In still other embodiments, the contacting is performed after the protein has been eluted from the chromatography column but before any further processing. Essentially, the contacting may be performed at any stage during preparation, purification, storage or formulation of the antibody. In some embodiments, partial purification by affinity chromatography (e.g., Protein A chromatography) may be conducted prior to the contacting.

The contacting may be also performed with antibodies attached to a stationary phase of a chromatographic columns, while the reducing agent are a part of the mobile phase; In this case the contacting may be performed as a part of chromatographic purification procedure. Examples of representative chromatographic refolding processes may include size exclusion (SEC); solvent exchange during reversible adsorption on protein A column; hydrophobic interaction chromatography (HIC); immobilized metal affinity chromatography (IMAC); reversed-phase chromatography (RPC); use of immobilized folding catalyst, such as GroE1, GroES or other proteins with folding properties. The on-column refolding is attractive because it is easily automated using commercially available preparative chromatographic systems. The refolding on column of recombinant proteins produced in microbial cell was recently reviewed in (Li et al., 2004).

If the contacting step is performed on a partially or highly purified preparation of recombinant protein, the contacting step can be performed for as short as about 1 hour to about 4 hours, and as long as about 6 hours to about 4 days. It has been found that a contacting step of about 2 to about 48 hours, or about 16 hours works well. The contacting step can also take place during another step, such as on a solid phase or during filtering or any other step in purification.

The methods of the invention can be performed over a wide temperature range. For example, the methods of the invention have been successfully carried out at temperatures from about 4 degrees C. to about 37 degrees C., however the best results were achieved at lower temperatures. A typical temperature for contacting a partially or fully purified preparation of the recombinant protein is about 4 degrees C. to about 25 degrees C. (ambient), or preferably at 23 degrees C., but can also be performed at lower temperatures and at higher temperature.

In addition, it is contemplated that the method may be performed at high pressure. Previously, high hydrostatic pressures (1000-2000 bar), combined with low, nondenaturing concentrations of guanidine hydrochloride below 1M has been used to disaggregate (solubilize) and refold several denatured proteins produced by E. coli as inclusion bodies that included human growth hormone and lysozyme, and b-lactamase (St John et al., Proc Natl Acad Sci USA, 96:13029-13033 (1999)). B-lactamase was refolded at high yields of active protein, even without added GdmHCl. In another study (Seefeldt et al., Protein Sci, 13:2639-2650 (2004)), the refolding yield of mammalian cell produced protein bikunin obtained with high pressure modulated refolding at 2000 bas was 70% by RP HPLC, significantly higher than the value of 55% (by RP-HPLC) obtained with traditional guanidine hydrochloride “dilution-refolding”. These findings indicate that high hydrostatic pressure facilitates disruption of inter- and intra-molecular interactions, leading to protein unfolding and disaggregation. The interaction of the high pressure on protein is similar to the interaction of proteins with chaotropic agents. Thus, it is contemplated that in the methods of the invention, instead of using chaotropic agents, high pressure is used for protein unfolding. Of course, a combination of high pressure and chaotropic agents also may be used in some instances.

The preparation of recombinant antibody/protein can be contacted with the reducing agent in various volumes as appropriate. For example, the methods of the invention have been carried out successfully at the analytical laboratory-scale (1-50 mL), preparative-scale (50 mL-10 L) and manufacturing-scale (10 L or more). The methods of the invention can be carried out on both small and large scale with reproducibility. As such, the concentration of antibody may be an industrial quantity (in terms of weight in grams) (e.g., an industrial amount of a specific IgG) or alternatively may be in milligram quantities. In specific embodiments, the concentration of the recombinant antibody in the reaction mixture is from about 1 mg/ml and about 50 mg/ml, more specifically, 10 mg/ml, 15 mg/ml or 20 mg/ml. The recombinant IgG1 molecules in these concentrations are particularly contemplated.

In certain embodiments, the proteins produced using media contain reducing agent are further processed in a separate processing step which employs chaotropic denaturants such as, for example, sodium dodecyl sulfate (SDS), urea or guanidium hydrochloride (GuHCl). Significant amounts of chaotropic agents are needed to observe perceptible unfolding. In some embodiments the processing step uses between 0.1M and 2 M chaotrope that produces an effect equivalent to the use of 0.1 M to 2M guanidine hydrochloride. In a specific embodiment, the oxidative refolding is achieved in the presence of approximately 1.0 M guanidine hydrochloride or an amount of other chaotropic agent that produces the same or similar amount of refolding as 1M guanidine hydrochloride. In some embodiments, the methods use between about 1.5 M and 0.5 M chaotrope. The amount of chaotropic agent used is based on the structural stability of the protein in the presence of the said chaotrope. One needs to have enough chaotrope present to perturb the local tertiary structure and/or quaternary structure of domain interactions of the protein, but less than that required to fully unfold secondary structure of the molecule and/or individual domains. To determine the point at which a protein will start to unfold by equilibrium denaturation, one practiced in the art would titrate a chaotrope into a solution containing the protein and monitor structure by a technique such as circular dichroism or fluorescence. There are other parameters that could be used to unfold or slightly perturb the structure of a protein that may be used instead of a chaotrope. Temperature and pressure are two fundamental parameters that have been previously used to alter the structure of a protein and may be used in place of a chaotropic agent while contacting with a redox agent. The inventors contemplate that any parameter that has been shown to denature or perturb a protein structure may be used by a person practiced in the art in place of a chaotropic agent.

Disulfide exchange can be quenched in any way known to those of skill in the art. For example, the reducing agent can be removed or its concentration can be reduced through a purification step, and/or it can be chemically inactivated by, e.g., acidifying the solution. Typically, when the reaction is quenched by acidification, the pH of the solution containing the reducing agent will be brought down below pH 7. In some embodiment, the pH is brought to below pH 6. Generally, the pH is reduced to between about pH 2 and about pH 6.

In some embodiments, removing the reducing agent may be conducted by dialysis, buffer exchange or any chromatography method described herein.

Preferentially Enriched (or Increased)

The term by “preferentially enriched (or increased)” means an increase in relative abundance of a desired form, or increase in relative proportion of a desired form, or increase the population of a desired form (structural isoform). In some embodiments, the methods described herein increase relative abundance of an antibody structural isoform such as an antibody having at least one disulfide bond formed between amino acid residues outside of the hinge region. In one embodiment, said at least one disulfide bond is formed between the amino acid residues at position 191 according to EU numbering in the respective CH1 regions of the first antigen-binding domain and the second antigen-binding domain. In certain embodiment, said methods produce a homogenous antibody preparation having at least 50%, 60%, 70%, 80%, 90%, preferably at least 95% molar ratio of said antibody having at least one disulfide bond formed outside of the hinge region.

Homogeneous

A “homogeneous” population of an antibody means an antibody population that comprises largely a single form of the antibody, for example, at least 50%, 60%, 70%, 80% or more, preferably at least 90%, 95%, 96%, 97%, 99% or 100% of the antibody in the solution or composition is in the properly folded form. Similarly, a “homogeneous” population of an antibody having at least one disulfide bond formed outside of the hinge region means a population of said antibody which comprises largely a single, properly folded form, for example, at least 50%, 60%, 70%, 80% or more, preferably at least 90%, 95%, 96%, 97%, 99% or 100% molar ratio of said antibody having at least one disulfide bond formed outside of the hinge region. In one preferred embodiment, said “homogeneous” population of an antibody comprises at least one disulfide bond which is formed between the amino acid residues at position 191 according to EU numbering in the respective CH1 regions of the first antigen-binding domain and the second antigen-binding domain (i.e. “paired cysteines” at the position 191 according to EU number in the CH1 region).

In preferred embodiments, the methods of the present invention produce a homogeneous antibody population or a homogeneous antibody preparation by the steps described herein.

Determining whether an antibody population is homogenous, and the relative abundance or proportions of a conformation of a protein/antibody in a mixture, can be done using any of a variety of analytical and/or qualitative techniques. If the two conformations resolve differently during separation techniques such as chromatography, electrophoresis, filtering or other purification technique, then the relative proportion of a conformation in the mixture can be determined using such purification techniques. For example, at least two different conformations of the recombinant IgG could be resolved by way of hydrophobic interaction chromatography. Further, since far UV Circular Dichroism has been used to estimate secondary structure composition of proteins (Perczel et al., 1991, Protein Engrg. 4:669-679), such a technique can determine whether alternative conformations of a protein are present. Still another technique used to determine conformation is fluorescence spectroscopy which can be employed to ascertain complementary differences in tertiary structure assignable to tryptophan and tyrosine fluorescence. Other techniques that can be used to determine differences in conformation and, hence, the relative proportions of a conformation, are on-line SEC to measure aggregation status, differential scanning calorimetry to measure melting transitions (Tm's) and component enthalpies, and chaotrope unfolding. Yet another technique that can be used to determine differences in conformation and, hence, the relative proportions of a conformation is LC/MS detection to determine the heterogeneity of the protein.

Alternatively, if there is a difference in activity between the conformations of the antibody/protein, determining the relative proportion of a conformation in the mixture can be done by way of an activity assay (e.g., binding to a ligand, enzymatic activity, biological activity, etc.). Biological activity of the protein also could be used. Alternatively, the binding assays can be used in which the activity is expressed as activity units/mg of protein.

In some embodiments described in detail herein below, the invention uses IEC chromatography, to determine the heterogeneity of the antibody/protein. In such a case, the antibody is purified or considered to be “homogenous”, which means that no polypeptide peaks or fractions corresponding to other polypeptides are detectable upon analysis by IEC chromatography. In certain embodiments, the antibody is purified or considered to be “homogenous” such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, and/or (if the polypeptide is radiolabeled) by auto radiography.

Herein, examples of conditions of SDS-PAGE analysis are as follows. Non-reducing SDS-PAGE was performed using 4-20% Mini-PROTEAN (registered trademark) TGX Stain-Free™ Precast Gels (Bio-Rad) with 1×Tris/Glycine/SDS running buffer (Bio-Rad). Monoclonal antibody samples were heated at 70 degrees C. for 10 min. 0.2 microgram was loaded and electrophoresis was conducted at 200 V for 90 min. Proteins were visualized with Chemidoc Imaging System (Bio-Rad). Percentage of individual band is analyzed by the Image Lab software version 6.0 (Bio-Rad), in which % intensity of the individual band (e.g. faster migration (Lower band) and slower migration (Upper band)) were calculated by intensity of the band divided by the sum of these two bands. Then, the gel may be stained with CBB, and the gel image may be captured, and the bands may be quantified using an imaging device. In the gel image, several, for example, two bands, i.e., “upper band” and “lower band”, may be observed for an antibody variant sample. In this case, the molecular weight of the upper band may correspond to that of the parent antibody (before modification). Structural changes such as crosslinking via disulfide bonds of Fabs may be caused by cysteine substitution, which may result in the change in electrophoretic mobility. In this case, the lower band may be considered to correspond to the antibody having one or more engineered disulfide bond(s) formed between the CH1 regions. Antibody variant samples with additional cysteine substitutions may show a higher lower band to upper band ratio, compared to control samples. Additional cysteine substitutions may enhance/promote disulfide bond crosslinking of Fabs; and may increase the percentage or structural homogeneity of an antibody preparation having an engineered disulfide bond formed at a mutated position; and may decrease the percentage of an antibody preparation having no engineered disulfide bond formed at the mutated position.

Methods for Capturing and/or Removing a Target Antibody from an Antibody Preparation

The present disclosure provides methods for capturing and/or removing a target antibody from an antibody preparation.

In an aspect, the present disclosure provides a method for capturing and/or removing a target antibody from an antibody preparation, comprising the steps of:
a) contacting the antibody preparation comprising the target antibody with an antigen-binding molecule immobilized on a support; and
b) allowing capture of the target antibody by specific binding to the antigen-binding molecule;
wherein said antibody comprises at least two Fabs from an IgG (preferably human IgG or human IgG1), and said antibody preparation comprises two antibody structural isoforms which differ by a disulfide bond formed between the two Fabs at the CH1 domain; and
wherein said antigen-binding molecule specifically binds and captures the target antibody which does not comprise the disulfide bond.

In an aspect, the antigen-binding molecule binds to the target antibody at an epitope which is only accessible to the antigen-binding molecule when the target antibody does not have the disulfide bond.

In an aspect, the disulfide bond is a disulfide bond formed between the two Fabs of the antibody at position 191 according to EU numbering in the CH1 domain.

In an aspect, the antigen-binding molecule that specifically binds the target antibody is an antibody which comprises any one selected from the group consisting of the following:
(a1) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 182, the light chain CDR 2 of SEQ ID NO: 186 and the light chain CDR 3 of SEQ ID NO: 190;
(a2) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 183, the light chain CDR 2 of SEQ ID NO: 187 and the light chain CDR 3 of SEQ ID NO: 191;
(a3) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 184, the light chain CDR 2 of SEQ ID NO: 188 and the light chain CDR 3 of SEQ ID NO: 192;
(a4) the heavy chain CDR 1 of SEQ ID NO: 169, the heavy chain CDR 2 of SEQ ID NO: 173, the heavy chain CDR 3 of SEQ ID NO: 177, the light chain CDR 1 of SEQ ID NO: 185, the light chain CDR 2 of SEQ ID NO: 189 and the light chain CDR 3 of SEQ ID NO: 193;
(a5) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 115, the light chain CDR 2 of SEQ ID NO: 124 and the light chain CDR 3 of SEQ ID NO: 134;
(a6) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 116, the light chain CDR 2 of SEQ ID NO: 125 and the light chain CDR 3 of SEQ ID NO: 135;
(a7) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 118, the light chain CDR 2 of SEQ ID NO: 128 and the light chain CDR 3 of SEQ ID NO: 137;
(a8) an antibody that binds to the same epitope of the antibody comprising any one of (a1) to (a7); and
(a9) an antibody that competes with the binding of the antibody comprising any one of (a1) to (a7).

In an aspect, the antigen-binding molecule that specifically binds the target antibody is an antibody which comprises any one selected from the group consisting of the following:
(a1) a heavy chain variable region comprising an amino acid sequence of SEQ ID NO: 162, and a light chain variable region comprising an amino acid sequence of SEQ ID NO: 178;
(a2) a heavy chain variable region comprising an amino acid sequence of SEQ ID NO: 163, and a light chain variable region comprising an amino acid sequence of SEQ ID NO: 179;
(a3) a heavy chain variable region comprising an amino acid sequence of SEQ ID NO: 164, and a light chain variable region comprising an amino acid sequence of SEQ ID NO: 180;
(a4) a heavy chain variable region comprising an amino acid sequence of SEQ ID NO: 165, and a light chain variable region comprising an amino acid sequence of SEQ ID NO: 181;
(a5) a heavy chain variable region comprising an amino acid sequence of SEQ ID NO: 162, and a light chain variable region comprising an amino acid sequence of SEQ ID NO: 196;
(a6) a heavy chain variable region comprising an amino acid sequence of SEQ ID NO: 163, and a light chain variable region comprising an amino acid sequence of SEQ ID NO: 197;
(a7) a heavy chain variable region comprising an amino acid sequence of SEQ ID NO: 164, and a light chain variable region comprising an amino acid sequence of SEQ ID NO: 198;
(a8) an antibody that binds to the same epitope of the antibody comprising any one of (a1) to (a7); and
(a9) an antibody that competes with the binding of the antibody comprising any one of (a1) to (a7).

In an aspect, the target antibody comprises five polypeptide chains in any one of the combination selected from (a1) to (a15) below:
(a1) a polypeptide chain comprising an amino acid sequence of SEQ ID NO: 201 (chain 1), a polypeptide chain comprising an amino acid sequence of SEQ ID NO: 206 (chain 2), a polypeptide chain comprising an amino acid sequence of SEQ ID NO: 208 (chain 3) and two polypeptide chains each comprising an amino acid sequence of SEQ ID NO: 214 (chain 4 & chain 5);
(a2) a polypeptide chain comprising an amino acid sequence of SEQ ID NO: 203 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NOs: 214 (chain 4 & chain 5);
(a3) a polypeptide chain comprising an amino acid sequence of SEQ ID NO: 204 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a4) a polypeptide chain comprising an amino acid sequence of SEQ ID NO: 205 (chain 1), a polypeptide chain comprising an amino acid sequence of SEQ ID NO: 206 (chain 2), a polypeptide chain comprising an amino acid sequence of SEQ ID NO: 209 (chain 3) and two polypeptide chains each comprising an amino acid sequence of SEQ ID NO: 214 (chain 4 & chain 5);
(a5) a polypeptide chain comprising an amino acid sequence of SEQ ID NO: 216 (chain 1), a polypeptide chain comprising an amino acid sequence of SEQ ID NO: 206 (chain 2), a polypeptide chain comprising an amino acid sequence of SEQ ID NO: 229 (chain 3) and two polypeptide chains each comprising an amino acid sequence of SEQ ID NO: 214 (chain 4 & chain 5);
(a6) a polypeptide chain comprising an amino acid sequence of SEQ ID NO: 217 (chain 1), a polypeptide chain comprising an amino acid sequence of SEQ ID NO: 206 (chain 2), a polypeptide chain comprising an amino acid sequence of SEQ ID NO: 210 (chain 3) and two polypeptide chains each comprising an amino acid sequence of SEQ ID NO: 214 (chain 4 & chain 5);
(a7) a polypeptide chain comprising an amino acid sequence of SEQ ID NO: 219 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a8) a polypeptide chain comprising an amino acid sequence of SEQ ID NO: 220 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a9) a polypeptide chain comprising an amino acid sequence of SEQ ID NO: 221 (chain 1), a polypeptide chain comprising an amino acid sequence of SEQ ID NO: 206 (chain 2), a polypeptide chain comprising an amino acid sequence of SEQ ID NO: 211 (chain 3) and two polypeptide chains each comprising an amino acid sequence of SEQ ID NO: 214 (chain 4 & chain 5);
(a10) a polypeptide chain comprising an amino acid sequence of SEQ ID NO: 222 (chain 1), a polypeptide chain comprising an amino acid sequence of SEQ ID NO: 206 (chain 2), a polypeptide chain comprising an amino acid sequence of SEQ ID NO: 230 (chain 3) and two polypeptide chains each comprising an amino acid sequence of SEQ ID NO: 214 (chain 4 & chain 5);
(a11) a polypeptide chain comprising an amino acid sequence of SEQ ID NO: 223 (chain 1), a polypeptide chain comprising an amino acid sequence of SEQ ID NO: 206 (chain 2), a polypeptide chain comprising an amino acid sequence of SEQ ID NO: 212 (chain 3) and two polypeptide chains each comprising an amino acid sequence of SEQ ID NO: 215 (chain 4 & chain 5);
(a12) a polypeptide chain comprising an amino acid sequence of SEQ ID NO: 225 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a13) a polypeptide chain comprising an amino acid sequence of SEQ ID NO: 226 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5);
(a14) a polypeptide chain comprising an amino acid sequence of SEQ ID NO: 227 (chain 1), a polypeptide chain comprising an amino acid sequence of SEQ ID NO: 206 (chain 2), a polypeptide chain comprising an amino acid sequence of SEQ ID NO: 213 (chain 3) and two polypeptide chains each comprising an amino acid sequence of SEQ ID NO: 215 (chain 4 & chain 5); and
(a15) a polypeptide chain comprising an amino acid sequence of SEQ ID NO: 228 (chain 1), a polypeptide chain comprising an amino acid sequence of SEQ ID NO: 206 (chain 2), a polypeptide chain comprising an amino acid sequence of SEQ ID NO: 231 (chain 3) and two polypeptide chains each comprising an amino acid sequence of SEQ ID NO: 215 (chain 4 & chain 5);
and wherein, preferably the five polypeptide chains (chain 1 to chain 5) connect and/or associate with each other according to the orientation shown in FIG. 1(a).

Conformation-Specific Antibodies

The present disclosure provides conformation-specific antibodies that specifically binds to a target antibody only when the target antibody does not have engineered disulfide bond between the two Fabs, e.g. at CH1 region (“unpaired cysteines” form). In an aspect, epitope(s) is/are not accessible to the conformation-specific antibodies when the target antibody has engineered disulfide bond (“paired cysteine” form) due to e.g. steric hindrance or reduced distance between the two Fabs caused by the engineered disulfide bond.

In an aspect, the conformation-specific antibody (antigen-binding molecule that specifically binds the target antibody) comprises any one selected from the group consisting of the following:
(a1) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 182, the light chain CDR 2 of SEQ ID NO: 186 and the light chain CDR 3 of SEQ ID NO: 190;
(a2) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 183, the light chain CDR 2 of SEQ ID NO: 187 and the light chain CDR 3 of SEQ ID NO: 191;
(a3) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 184, the light chain CDR 2 of SEQ ID NO: 188 and the light chain CDR 3 of SEQ ID NO: 192;
(a4) the heavy chain CDR 1 of SEQ ID NO: 169, the heavy chain CDR 2 of SEQ ID NO: 173, the heavy chain CDR 3 of SEQ ID NO: 177, the light chain CDR 1 of SEQ ID NO: 185, the light chain CDR 2 of SEQ ID NO: 189 and the light chain CDR 3 of SEQ ID NO: 193;
(a5) the heavy chain CDR 1 of SEQ ID NO: 166, the heavy chain CDR 2 of SEQ ID NO: 170, the heavy chain CDR 3 of SEQ ID NO: 174, the light chain CDR 1 of SEQ ID NO: 115, the light chain CDR 2 of SEQ ID NO: 124 and the light chain CDR 3 of SEQ ID NO: 134;
(a6) the heavy chain CDR 1 of SEQ ID NO: 167, the heavy chain CDR 2 of SEQ ID NO: 171, the heavy chain CDR 3 of SEQ ID NO: 175, the light chain CDR 1 of SEQ ID NO: 116, the light chain CDR 2 of SEQ ID NO: 125 and the light chain CDR 3 of SEQ ID NO: 135;
(a7) the heavy chain CDR 1 of SEQ ID NO: 168, the heavy chain CDR 2 of SEQ ID NO: 172, the heavy chain CDR 3 of SEQ ID NO: 176, the light chain CDR 1 of SEQ ID NO: 118, the light chain CDR 2 of SEQ ID NO: 128 and the light chain CDR 3 of SEQ ID NO: 137;
(a8) an antibody that binds to the same epitope of the antibody comprising any one of (a1) to (a7); and
(a9) an antibody that competes with the binding of the antibody comprising any one of (a1) to (a7).

In an aspect, the conformation-specific antibody (antigen-binding molecule that specifically binds the target antibody) comprises any one selected from the group consisting of the following:
(a1) a heavy chain variable region comprising an amino acid sequence of SEQ ID NO: 162, and a light chain variable region comprising an amino acid sequence of SEQ ID NO: 178;
(a2) a heavy chain variable region comprising an amino acid sequence of SEQ ID NO: 163, and a light chain variable region comprising an amino acid sequence of SEQ ID NO: 179;
(a3) a heavy chain variable region comprising an amino acid sequence of SEQ ID NO: 164, and a light chain variable region comprising an amino acid sequence of SEQ ID NO: 180;
(a4) a heavy chain variable region comprising an amino acid sequence of SEQ ID NO: 165, and a light chain variable region comprising an amino acid sequence of SEQ ID NO: 181;
(a5) a heavy chain variable region comprising an amino acid sequence of SEQ ID NO: 162, and a light chain variable region comprising an amino acid sequence of SEQ ID NO: 196;
(a6) a heavy chain variable region comprising an amino acid sequence of SEQ ID NO: 163, and a light chain variable region comprising an amino acid sequence of SEQ ID NO: 197;
(a7) a heavy chain variable region comprising an amino acid sequence of SEQ ID NO: 164, and a light chain variable region comprising an amino acid sequence of SEQ ID NO: 198;
(a8) an antibody that binds to the same epitope of the antibody comprising any one of (a1) to (a7); and
(a9) an antibody that competes with the binding of the antibody comprising any one of (a1) to (a7).

In an aspect, the conformation-specific antibody (antigen-binding molecule that specifically binds the target antibody) specifically binds to CH1 of human IgG1.

In an aspect, the conformation-specific antibody (antigen-binding molecule that specifically binds the target antibody) does not specifically bind to CH1 of human IgG1 when a disulfide bond is formed between the CH1 domains of the two Fabs of human IgG1. In a further aspect, the disulfide bond is a disulfide bond formed between the two Fabs of the IgG1 at position 191 according to EU numbering in the CH1 domain.

In an aspect, the conformation-specific antibody (antigen-binding molecule that specifically binds the target antibody) does not bind to CH1 of human IgG4.

The present disclosure provides use of the conformation-specific antibodies (antigen-binding molecules that specifically bind the target antibody) in purification, analytical or quantification of an antibody-containing sample.

Antigen

As used herein, the term “antigen” refers to a site (e.g. a contiguous stretch of amino acids or a conformational configuration made up of different regions of noncontiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

The “first antigen” or the “second antigen” to which a first antigen-binding moiety and/or a second antigen-binding moiety binds is preferably, for example, an immunocyte surface molecule (e.g., a T cell surface molecule, an NK cell surface molecule, a dendritic cell surface molecule, a B cell surface molecule, an NKT cell surface molecule, an MDSC cell surface molecule, and a macrophage surface molecule), or an antigen expressed not only on tumor cells, tumor vessels, stromal cells, and the like but on normal tissues (integrin, tissue factor, VEGFR, PDGFR, EGFR, IGFR, MET chemokine receptor, heparan sulfate proteoglycan, CD44, fibronectin, DR5, TNFRSF, etc.).

As for the combination of the “first antigen” and the “second antigen”, preferably, any one of the first antigen and the second antigen is, for example, a molecule specifically expressed on a T cell, and the other antigen is a molecule expressed on the surface of a T cell or any other immunocyte. In another embodiment of the combination of the “first antigen” and the “second antigen”, preferably, any one of the first antigen and the second antigen is, for example, a molecule specifically expressed on a T cell, and the other antigen is a molecule that is expressed on an immunocyte and is different from the preliminarily selected antigen.

Specific examples of the molecule specifically expressed on a T cell include CD3 and T cell receptors. Particularly, CD3 is preferred. In the case of, for example, human CD3, a site in the CD3 to which the antigen-binding molecule of the present invention binds may be any epitope present in a gamma chain, delta chain, or epsilon chain sequence constituting the human CD3. Particularly, an epitope present in the extracellular region of an epsilon chain in a human CD3 complex is preferred. The polynucleotide sequences of the gamma chain, delta chain, and epsilon chain structures constituting CD3 are NM_000073.2, NM_000732.4, and NM_000733.3, and the polypeptide sequences thereof are NP_000064.1, NP_000723.1, and NP_000724.1 (RefSeq registration numbers). Examples of the other antigen include Fc gamma receptors, TLR, lectin, IgA, immune checkpoint molecules, TNF superfamily molecules, TNFR superfamily molecules, and NK receptor molecules.

In one embodiment, the first antigen is a molecule specifically expressed on a T cell, preferably a T cell receptor complex molecule such as CD3, more preferably human CD3. In another embodiment, the second antigen is a molecule expressed on a T cell or any other immune cell, preferably a cell surface modulator on an immune cell, more preferably a costimulatory molecule expressed on a T cell, and even more preferably a protein of “TNF superfamily” or the “TNF receptor superfamily” including not limited to human CD137 (4-1BB), CD137L, CD40, CD40L, OX40, OX40L, CD27, CD70, HVEM, LIGHT, RANK, RANKL, CD30, CD153, GITR, and GITRL. In one preferred embodiment, the first antigen is CD3 and the second antigen is CD137. Here, the first antigen and the second antigen are defined interchangeably.

The term “CD137” herein, also called 4-1BB, is a member of the tumor necrosis factor (TNF) receptor family. Examples of factors belonging to the TNF superfamily or the TNF receptor superfamily include CD137, CD137L, CD40, CD40L, OX40, OX40L, CD27, CD70, HVEM, LIGHT, RANK, RANKL, CD30, CD153, GITR, and GITRL.

In some embodiments of the present invention, the antigen-binding molecule of the present invention further comprises a third antigen-binding moiety which binds to a “third antigen” that is different from the “first antigen” and the “second antigen” mentioned above. The third antigen-binding domain binding to a third antigen of the present invention can be an antigen-binding moiety that recognizes an arbitrary antigen. The third antigen-binding moiety binding to a third antigen of the present invention can be an antigen-binding moiety that recognizes a molecule specifically expressed in a cancer tissue.

In the present invention, a third antigen-binding moiety in the antigen-binding molecule of the present invention binds to a “third antigen” that is different from the “first antigen” and the “second antigen”. In some embodiments, the third antigen is derived from humans, mice, rats, monkeys, rabbits, or dogs. In some embodiments, the third antigen is a molecule specifically expressed on the cell or the organ derived from humans, mice, rats, monkeys, rabbits, or dogs. The third antigen is preferably, a molecule not systemically expressed on the cell or the organ. The third antigen is preferably, for example, a tumor cell-specific antigen and also includes an antigen expressed in association with the malignant alteration of cells as well as an abnormal sugar chain that appears on cell surface or a protein molecule during the malignant transformation of cells. Specific examples thereof include ALK receptor (pleiotrophin receptor), pleiotrophin, KS 1/4 pancreatic cancer antigen, ovary cancer antigen (CA125), prostatic acid phosphate, prostate-specific antigen (PSA), melanoma-associated antigen p97, melanoma antigen gp75, high-molecular-weight melanoma antigen (HMW-MAA), prostate-specific membrane antigen, carcinoembryonic antigen (CEA), polymorphic epithelial mucin antigen, human milk fat globule antigen, colorectal tumor-associated antigen (e.g., CEA, TAG-72, CO17-1A, GICA 19-9, CTA-1, and LEA), Burkitt's lymphoma antigen 38.13, CD19, human B lymphoma antigen CD20, CD33, melanoma-specific antigen (e.g., ganglioside GD2, ganglioside GD3, ganglioside GM2, and ganglioside GM3), tumor-specific transplantation antigen (TSTA), T antigen, virus-induced tumor antigen (e.g., envelope antigens of DNA tumor virus and RNA tumor virus), colon CEA, oncofetal antigen alpha-fetoprotein (e.g., oncofetal trophoblastic glycoprotein 5T4 and oncofetal bladder tumor antigen), differentiation antigen (e.g., human lung cancer antigens L6 and L20), fibrosarcoma antigen, human T cell leukemia-associated antigen Gp37, newborn glycoprotein, sphingolipid, breast cancer antigen (e.g., EGFR (epithelial growth factor receptor)), NY-BR-16, NY-BR-16 and HER2 antigen (p185HER2), polymorphic epithelial mucin (PEM), malignant human lymphocyte antigen APO-1, differentiation antigen such as I antigen found in fetal erythrocytes, primary endoderm I antigen found in adult erythrocytes, I (Ma) found in embryos before transplantation or gastric cancer, M18 found in mammary gland epithelium, M39, SSEA-1 found in bone marrow cells, VEP8, VEP9, My1, VIM-D5, D156-22 found in colorectal cancer, TRA-1-85 (blood group H), SCP-1 found in testis and ovary cancers, C14 found in colon cancer, F3 found in lung cancer, AH6 found in gastric cancer, Y hapten, Ley found in embryonic cancer cells, TL5 (blood group A), EGF receptor found in A431 cells, El series (blood group B) found in pancreatic cancer, FC10.2 found in embryonic cancer cells, gastric cancer antigen, CO-514 (blood group Lea) found in adenocarcinoma, NS-10 found in adenocarcinoma, CO-43 (blood group Leb), G49 found in A431 cell EGF receptor, MH2 (blood group ALeb/Ley) found in colon cancer, 19.9 found in colon cancer, gastric cancer mucin, T5A7 found in bone marrow cells, R24 found in melanoma, 4.2, GD3, D1.1, OFA-1, GM2, OFA-2, GD2, and M1:22:25:8 found in embryonic cancer cells, SSEA-3 and SSEA-4 found in 4-cell to 8-cell embryos, cutaneous T cell lymphoma-associated antigen, MART-1 antigen, sialyl Tn (STn) antigen, colon cancer antigen NY-CO-45, lung cancer antigen NY-LU-12 variant A, adenocarcinoma antigen ART1, paraneoplastic associated brain-testis-cancer antigen (onconeuronal antigen MA2 and paraneoplastic neuronal antigen), neuro-oncological ventral antigen 2 (NOVA2), blood cell cancer antigen gene 520, tumor-associated antigen CO-029, tumor-associated antigen MAGE-C1 (cancer/testis antigen CT7), MAGE-B1 (MAGE-XP antigen), MAGE-B2 (DAM6), MAGE-2, MAGE-4a, MAGE-4b MAGE-X2, cancer-testis antigen (NY-EOS-1), YKL-40, and any fragment of these polypeptides, and modified structures thereof (aforementioned modified phosphate groups, sugar chains, etc.), EpCAM, EREG, CA19-9, CA15-3, sialyl SSEA-1 (SLX), HER2, PSMA, CEA, and CLEC12A.

In one preferred embodiment, the third antigen is Glypican-3 (GPC3). In yet another embodiment, the third antigen is DLL3 (Delta-like 3).

The term “DLL3”, as used herein, refers to any native DLL3 (Delta-like 3) from any vertebrate source, including mammals such as primates (e.g. humans) and rodents (e.g., mice and rats), unless otherwise indicated. The term encompasses “full-length” unprocessed DLL3 as well as any form of DLL3 that results from processing in the cell. The term also encompasses naturally occurring variants of DLL3, e.g., splice variants or allelic variants. The amino acid sequence of an exemplary human DLL3 is known as NCBI Reference Sequence (RefSeq) NM_016941.3, and the amino acid sequence of an exemplary cynomolgus DLL3 is known as NCBI Reference Sequence XP_005589253.1, and the amino acid sequence of an exemplary mouse DLL3 is known as NCBI Reference Sequence NM_007866.2.

The human DLL3 protein comprises a transmembrane (TM) region and an intracellular domain on the C-terminal side, and a DSL (Notch) domain on the N-terminal side. In addition, DLL3 has an EGF domain comprising six regions, EGF1 to EGF6 from the N-terminal side to the C-terminal side. In some embodiments, the multispecific antigen-binding molecules or the DLL3 antigen binding moiety of the present invention bind to an epitope within the extracellular domain (ECD), i.e., the domain from the N-terminus to immediately before the TM region, but not to the TM region or the C-terminal intracellular domain. The multispecific antigen-binding molecules or the DLL3 antigen binding moiety of the present invention may bind to an epitope within any of the above-mentioned domains/regions within the ECD. In preferred embodiments, the multispecific antigen-binding molecules or the DLL3 antigen binding moiety of the present invention bind to an epitope within the region from EGF6 to immediately before the TM region. More specifically, the multispecific antigen-binding molecules or the DLL3 antigen binding moiety of the present invention may bind to an epitope within the regions defined in SEQ ID NO: 89 in human DLL3. In some embodiments, the molecules/antibodies of the present invention bind to the EGF1, EGF2, EGF3, EGF4, EGF5, or EGF6 region or a region from EGF6 to immediately before the TM region of human DLL3, or an epitope within the EGF1, EGF2, EGF3, EGF4, EGF5, or EGF6 region or a region from EGF6 to immediately before the TM region of human DLL3.

In human DLL3, the above-mentioned domains/regions have the following amino acid residues (see, e.g., http://www.uniprot.org/uniprot/Q9NYJ7 or WO2013/126746):

Extracellular domain (ECD): amino acid residues at positions 1 to 492;

DSL domain: amino acid residues at positions 176 to 215;

EGF domain: amino acid residues at positions 216 to 465;

EGF1 region: amino acid residues at positions 216 to 249;

EGF2 region: amino acid residues at positions 274 to 310;

EGF3 region: amino acid residues at positions 312 to 351;

EGF4 region: amino acid residues at positions 353 to 389;

EGF5 region: amino acid residues at positions 391 to 427;

EGF6 region: amino acid residues at positions 429 to 465;

The region from EGF6 to immediately before the TM region: amino acid residues at positions 429 to 492;

TM region: amino acid residues at positions 493 to 513; and

C-terminal intracellular domain: amino acid residues at positions 516 to 618 (or 516 to 587 in some isoforms). The amino acid positions mentioned above also refers to the amino acid positions in the amino acid sequence shown in SEQ ID NO: 90.

Thus, the multispecific antigen-binding molecules or the DLL3 antigen binding moiety of the present invention may bind to an above-mentioned region/domain having the amino acid residues at the above-mentioned positions in human DLL3. That is, the multispecific antigen-binding molecules or the DLL3 antigen binding moiety of the present invention may bind to an epitope within the above-mentioned region/domain having the amino acid residues at the above-mentioned positions in human DLL3.

The DLL3 protein used in the present invention may be a DLL3 protein having the sequence described above or may be a modified protein having a sequence derived from the sequence described above by the modification of one or more amino acids. Examples of the modified protein having a sequence derived from the sequence described above by the modification of one or more amino acids can include polypeptides having 70% or more, preferably 80% or more, more preferably 90% or more, even more preferably 95% or more identity with to the amino acid sequence described above. Alternatively, partial peptides of these DLL3 proteins may be used.

The DLL3 protein used in the present invention is not limited by its origin and is preferably a human or cynomolgus DLL3 protein.

In some embodiments, for the DLL3 protein, DLL3 ECD fragment proteins (or ECD variants) may be used. Depending on the site of truncation, the fragments/variants may comprise, from the N-terminal side to the C-terminal side, the DSL domain to EGF6, EGF1 to EGF6, EGF2 to EGF6, EGF3 to EGF6, EGF4 to EGF6, EGF5 and EGF6, or EGF6. The fragments/variants may further comprise a region spanning from immediately after the EGF6 region to immediately before the TM region. A Flag tag may be attached to the C terminus of the fragments/variants using a technique well-known in the art.

In certain embodiments the multispecific antigen binding molecule described herein binds to an epitope of CD3, CD137 or DLL3 that is conserved among the CD3, CD137 or DLL3 from different species. In certain embodiments the multispecific antigen binding molecule of the present application is a trispecific antigen binding molecule, i.e. it is capable of specifically binding to three different antigens-capable of binding to either one of CD3 or CD137 but does not bind to both antigens simultaneously, and is capable of specifically binding to DLL3.

In certain embodiments, the multispecific antigen binding molecule specifically binds to the whole or a portion of a partial peptide of CD3. In a particular embodiment CD3 is human CD3 or cynomolgus CD3, most particularly human CD3. In a particular embodiment the multispecific antigen binding molecule is cross-reactive for (i.e. specifically binds to) human and cynomolgus CD3. In some embodiments, the multispecific antigen binding molecule is capable of specific binding to the epsilon subunit of CD3, in particular the human CD3 epsilon subunit of CD3 which is shown in SEQ ID NO: 7 (NP_000724.1) (RefSeq registration numbers are shown within the parentheses). In some embodiments, the multispecific antigen binding molecule is capable of specific binding to the CD3 epsilon chain expressed on the surface of eukaryotic cells. In some embodiments, the multispecific antigen binding molecule binds to the CD3 epsilon chain expressed on the surface of T cells.

In certain embodiments, the CD137 is human CD137. In some embodiments, favorable examples of an antigen-binding molecule of the present invention include antigen-binding molecules that bind to the same epitope as the human CD137 epitope bound by the antibody selected from the group consisting of:
antibody that recognize a region comprising the SPCPPNSFSSAGGQRTCD ICRQCKGVFRTRKECSSTSNAECDCTPGFHCLGAGCSMCEQDCKQGQELTK KGC
sequence (SEQ ID NO: 21),
antibody that recognize a region comprising the DCTPGFHCLGAGCSMCEQDC KQGQELTKKGC sequence (SEQ ID NO: 35),
antibody that recognize a region comprising the LQDPCSNC PAGTFCDNNRNQICSPCPPNSFSSAGGQRTCDICRQCKGVFRTRKECSSTSNA EC
sequence (SEQ ID NO: 49), and
antibody that recognize a region comprising the LQDPCSNCPAGTFCDNNRN QIC sequence (SEQ ID NO: 105) in the human CD137 protein.

At Least One Disulfide Bond

In one aspect of the present invention, each of the first antigen-binding moiety and the second antigen-binding moiety comprises at least one cysteine residue (via mutation, substitution or insertion), preferably in the CH1 region, and said at least one cysteine residue is capable of forming at least one disulfide bond between the first antigen-binding moiety and the second antigen-binding moiety. In certain embodiments, the cysteine residue is present within a CH1 region of an antibody heavy chain constant region, and for example, it is present at a position selected from the group consisting of positions 119, 122, 123, 131, 132, 133, 134, 135, 136, 137, 139, 140, 148, 150, 155, 156, 157, 159, 160, 161, 162, 163, 165, 167, 174, 176, 177, 178, 190, 191, 192, 194, 195, 197, 213, and 214 according to EU numbering in the CH1 region. In one embodiment, each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.

In an embodiment of the above aspects, “at least one bond” to be formed linking the first antigen-binding moiety and the second antigen-binding moiety as described above can hold the two antigen binding moiety (i.e., the first antigen-binding moiety and the second antigen-binding moiety as described above) spatially close positions. By virtue of the linkage between the first antigen-binding moiety and the second antigen-binding moiety via the disulfide bond(s), the antigen-binding molecule of the present invention is capable of holding two antigen-binding moieties at closer positions than a control antigen-binding molecule, which differs from the antigen-binding molecule of the present invention only in that the control antigen-binding molecule does not have the additional bond(s) introduced between the two antigen-binding moieties. In some embodiments, the term “spatially close positions” or “closer positions” includes the meaning that the first antigen-binding domain and the second antigen-binding domain as described above hold in shortened distance and/or reduced flexibility.

As the results, the two antigen binding moieties (i.e., the first antigen-binding moiety and the second antigen-binding moiety as described above) of the antigen-binding molecule of the present invention binds to the antigens expressed on the same single cell. In other words, the respective two antigen-binding moieties (i.e., the first antigen-binding moiety and the second antigen-binding moiety as described above) of the antigen-binding molecule of the present invention do not bind to antigens expressed on different cells so as to cause a cross-linking the different cells. In the present application, such antigen-binding manner of the antigen-binding molecule of the present invention can be called as “cis-binding”, whereas the antigen-binding manner of an antigen-binding molecule which respective two antigen-binding moiety of the antigen-binding molecule bind to antigens expressed on different cells so as to cause a crosslinking the different cells can be called as “trans-binding”. In some embodiments, the antigen-binding molecule of the present invention predominantly binds to the antigens expressed on the same single cell in “cis-binding” manner.

In an embodiment of the above aspects, by virtue of the disulfide linkage between the first antigen-binding moiety and the second antigen-binding moiety via the disulfide bond(s) as described above, the antigen-binding molecule of the present invention is capable of reducing and/or preventing unwanted cross-linking and activation of immune cells (e.g., T-cells, NK cells, DC cells, or the like). That is, in some embodiments of the present invention, the first antigen-binding moiety of the antigen-binding molecule of the present invention binds to any signaling molecule expressed on an immune cell such as T-cell (e.g., the first antigen), and the second antigen-binding domain of the antigen-binding molecule of the present invention also binds to any signaling molecule expressed on an immune cell such as T-cell (e.g., the first antigen or the second antigen which is different from the first antigen). Thus, the first antigen-binding domain and the second antigen-binding domain of the antigen binding-molecule of the present invention can bind to either of the first or second signaling molecule expressed on the same single immune cell such as T cell (i.e., cis-binding manner) or on different immune cell such as T cells (i.e., trans-biding manner). When the first antigen-binding domain and the second antigen-binding domain bind to the signaling molecule expressed on different immune cells such as T-cells in trans-binding manner, those different immune cells such as T-cells are cross-linked, and, in certain situation, such crosslinking of immune cells such as T-cells may cause unwanted activation of the immune cells such as T-cells.

On the other hand, in the case of another embodiment of the antigen-binding molecule of the present invention, that is, an antigen-binding molecule comprising the first antigen-binding moiety and the second antigen-binding moiety, which are linked with each other via at least one disulfide bond in the CH1 region (position 191 according to EU numbering), both of the first antigen-binding domain and the second antigen-binding domain can binds to the signaling molecules expressed on the same single immune cells such as T cell in “cis-biding” manner, so that the crosslinking of different immune cells such as T-cells via the antigen-binding molecule can be reduced to avoid unwanted activation of immune cells.

In the instant application, the above-described feature, the at least one disulfide bond in the CH1 region (e.g. position 191 according to EU numbering) linking the first antigen-binding moiety and the second antigen-binding moiety may be described with the abbreviated term “LINC”. Using this abbreviation, in some embodiments, the above-described antigen-binding molecule of the present invention may be indicated as, e.g., “Dual/LINC”, “DLL3-Dual/LINC”, “paired cysteines form” or “GPC3-Dual/Dual (linc)” or the like. Antigen-binding molecules of which the first antigen-binding moiety and the second antigen-binding moiety that are not linked/yet to be linked with each other via at least one disulfide bond in the CH1 region (e.g. position 191 according to EU numbering) may be described with the abbreviated term “UnLINC” or “Dual-LINC-Ig with unpaired cysteines” or the like.

Hinge Region

The term “hinge region” denotes an antibody heavy chain polypeptide portion in a wild-type antibody heavy chain that joins the CH1 domain and the CH2 domain, e.g., from about position 216 to about position 230 according to the EU numbering system, or from about position 226 to about position 243 according to the Kabat numbering system. It is known that in a native IgG antibody, cysteine residue at position 220 according to EU numbering in the hinge region forms a disulfide bond with cysteine residue at position 214 in the antibody light chain. It is also known that between the two antibody heavy chains, disulfide bonds are formed between cysteine residues at position 226 and between cysteine residues at position 229 according to EU numbering in the hinge region. In general, a “hinge region” is defined as extending from human IgG1 from 216 to 238 (EU numbering) or from 226 to 251 (Kabat numbering). This hinge can be further divided into three different regions, an upper hinge, a central hinge and a lower hinge. In human IgG1 antibodies, these regions are generally defined as follows:

Upper hinge: 216-225 (EU numbering) or 226-238 (Kabat numbering),

Central hinge: 226-230 (EU numbering) or 239-243 (Kabat numbering),

Lower hinge: 231-238 (EU numbering) or 244-251 (Kabat numbering).

The hinge region of other IgG isotypes can be aligned with the IgG1 sequence by placing the first and last cysteine residues that form an interheavy chain SS bond in the same position (e.g., Brekke et al., 1995, Immunol (See Table 1 of Today 16: 85-90). A hinge region herein includes wild-type hinge regions as well as variants in which amino acid residue(s) in a wild-type hinge region is altered by substitution, addition, or deletion.

The term “disulfide bond formed between amino acids which are not in a hinge region” (or “disulfide bond formed between amino acids outside of a hinge region”) means disulfide bond formed, connected or linked through amino acids located in any antibody region which is outside of the “hinge region” defined above. For example, such disulfide bond is formed, connected or linked through amino acids in any position in an antibody other than in a hinge region (e.g., from about position 216 to about position 230 according to the EU numbering system, or from about position 226 to about position 243 according to the Kabat numbering system). In some embodiments, such disulfide bond is formed, connected or linked through amino acids located in a CH1 region, a CL region, a VL region, a VH region and/or a VHH region. In some embodiments, such disulfide bond is formed, connected or linked through amino acids located in positions 119 to 123, 131 to 140, 148 to 150, 155 to 167, 174 to 178, 188 to 197, 201 to 214, according to EU numbering, in the CH1 region. In some embodiments, such disulfide bond is formed, connected or linked through amino acids located in positions 119, 122, 123, 131, 132, 133, 134, 135, 136, 137, 138, 139, 140, 148, 150, 155, 156, 157, 159, 160, 161, 162, 163, 164, 165, 167, 174, 176, 177, 178, 188, 189, 190, 191, 192, 193, 194, 195, 196, 197, 201, 203, 205, 206, 207, 208, 211, 212, 213, 214 according to EU numbering, in the CH1 region. In some embodiments, such disulfide bond is formed, connected or linked through amino acids located in positions 188, 189, 190, 191, 192, 193, 194, 195, 196, and 197, according to EU numbering, in the CH1 region. In one preferred embodiment, such disulfide bond is formed, connected or linked through amino acids located in position 191, according to EU numbering, in the CH1 region.

Antigen Binding Domain

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, the antigen-binding domains contain both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab′)2”.

Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

HVR or CDR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

Capable of Binding to CD3 and CD137 but does not Bind to CD3 and CD137 at the Same Time

Whether the antibody variable region of the present invention is “capable of binding to CD3 and CD137” can be determined by a method known in the art.

This can be determined by, for example, an electrochemiluminescence method (ECL method) (BMC Research Notes 2011, 4: 281).

Specifically, for example, a low-molecular antibody composed of a region capable of binding to CD3 and CD137, for example, a Fab region, of a biotin-labeled antigen-binding molecule to be tested, or a monovalent antibody (antibody lacking one of the two Fab regions carried by a usual antibody) thereof is mixed with CD3 or CD137 labeled with sulfo-tag (Ru complex), and the mixture is added onto a streptavidin-immobilized plate. In this operation, the biotin-labeled antigen-binding molecule to be tested binds to streptavidin on the plate. Light is developed from the sulfo-tag, and the luminescence signal can be detected using Sector Imager 600 or 2400 (MSD K. K.) or the like to thereby confirm the binding of the aforementioned region of the antigen-binding molecule to be tested to CD3 or CD137.

Alternatively, this assay may be conducted by ELISA, FACS (fluorescence activated cell sorting), ALPHAScreen (amplified luminescent proximity homogeneous assay screen), the BIACORE method based on a surface plasmon resonance (SPR) phenomenon, etc. (Proc. Natl. Acad. Sci. USA (2006) 103 (11), 4005-4010).

Specifically, the assay can be conducted using, for example, an interaction analyzer Biacore (GE Healthcare Japan Corp.) based on a surface plasmon resonance (SPR) phenomenon. The Biacore analyzer includes any model such as Biacore T100, T200, X100, A100, 4000, 3000, 2000, 1000, 8K or C. Any sensor chip for Biacore, such as a CM7, CM5, CM4, CM3, C1, SA, NTA, L1, HPA, or Au chip, can be used as a sensor chip. Proteins for capturing the antigen-binding molecule of the present invention, such as protein A, protein G, protein L, anti-human IgG antibodies, anti-human IgG-Fab, anti-human L chain antibodies, anti-human Fc antibodies, antigenic proteins, or antigenic peptides, are immobilized onto the sensor chip by a coupling method such as amine coupling, disulfide coupling, or aldehyde coupling. CD3 or CD137 is injected thereon as an analyte, and the interaction is measured to obtain a sensorgram. In this operation, the concentration of CD3 or CD137 can be selected within the range of a few micro M to a few pM according to the interaction strength (e.g., KD) of the assay sample.

Alternatively, CD3 or CD137 may be immobilized instead of the antigen-binding molecule onto the sensor chip, with which the antibody sample to be evaluated is in turn allowed to interact. Whether the antibody variable region of the antigen-binding molecule of the present invention has binding activity against CD3 or CD137 can be confirmed on the basis of a dissociation constant (KD) value calculated from the sensorgram of the interaction or on the basis of the degree of increase in the sensorgram after the action of the antigen-binding molecule sample over the level before the action.

In some embodiments, binding activity or affinity of the antibody variable region of the present invention to the antigen of interest (i.e. CD3 or CD137) are assessed at 37 degrees Celsius (degrees C.) (for CD137) or 25 degrees C. (for CD3) using e.g., Biacore T200 instrument (GE Healthcare) or Biacore 8K instrument (GE Healthcare). Antihuman Fc (e.g., GE Healthcare) is immobilized onto all flow cells of a CM4 sensor chip using amine coupling kit (e.g, GE Healthcare). The antigen binding molecules or antibody variable regions are captured onto the anti-Fc sensor surfaces, then the antigen (CD3 or CD137) is injected over the flow cell. The capture levels of the antigen binding molecules or antibody variable regions may be aimed at 200 resonance unit (RU). Recombinant human CD3 or CD137 may be injected at 400 to 25 nM prepared by two-fold serial dilution, followed by dissociation. All antigen binding molecules or antibody variable regions and analytes are prepared in ACES pH 7.4 containing 20 mM ACES, 150 mM NaCl, 0.05% Tween 20, 0.005% NaN3. Sensor surface is regenerated each cycle with 3M MgCl2. Binding affinity are determined by processing and fitting the data to 1:1 binding model using e.g., Biacore T200 Evaluation software, version 2.0 (GE Healthcare) or Biacore Insight Evaluation software (GE Healthcare). The KD values are calculated for assessing the specific binding activity or affinity of the antigen binding domains of the present invention.

The ALPHAScreen is carried out by the ALPHA technology using two types of beads (donor and acceptor) on the basis of the following principle: luminescence signals are detected only when these two beads are located in proximity through the biological interaction between a molecule bound with the donor bead and a molecule bound with the acceptor bead. A laser-excited photosensitizer in the donor bead converts ambient oxygen to singlet oxygen having an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, singlet oxygen produced by the donor bead does not reach the acceptor bead. Thus, no chemiluminescent reaction occurs.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is injected on the surface of the sensor chip. Upon binding of the analyte to the ligand, the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). The amount of the analyte bound to the ligand captured on the sensor chip surface (amount of change in response on the sensorgram between before and after the interaction of the analyte) can be determined from the sensorgram. However, since the amount bound also depends on the amount of the ligand, the comparison must be performed under conditions where substantially the same amounts of the ligand are used. Kinetics, i.e., an association rate constant (ka) and a dissociation rate constant (kd), can be determined from the curve of the sensorgram, while affinity (KD) can be determined from the ratio between these constants. Inhibition assay is also preferably used in the BIACORE method. Examples of the inhibition assay are described in Proc. Natl. Acad. Sci. USA (2006) 103 (11), 4005-4010.

The term “does not bind to CD3 and CD137 (4-1BB) at the same time” or “does not bind to CD3 and CD137 (4-1BB) simultaneously” means that the antigen-binding moiety or antibody variable region of the present invention cannot bind to CD137 in a state bound with CD3 whereas the antigen-binding moiety or antibody variable region cannot bind to CD3 in a state bound with CD137. In this context, the phrase “not bind to CD3 and CD137 at the same time” also includes not cross-linking a cell expressing CD3 to a cell expressing CD137, or not binding to CD3 and CD137 each expressed on a different cell, at the same time. This phrase further includes the case where the variable region is capable of binding to both CD3 and CD137 at the same time when CD3 and CD137 are not expressed on cell membranes, as with soluble proteins, or both reside on the same cell, but cannot bind to CD3 and CD137 each expressed on a different cell, at the same time. Such an antibody variable region is not particularly limited as long as the antibody variable region has these functions. Examples thereof can include variable regions derived from an IgG-type antibody variable region by the alteration of a portion of its amino acids so as to bind to the desired antigen. The amino acid to be altered is selected from, for example, amino acids whose alteration does not cancel the binding to the antigen, in an antibody variable region binding to CD3 or CD137.

In this context, the phrase “expressed on different cells” merely means that the antigens are expressed on separate cells. The combination of such cells may be, for example, the same types of cells such as a T cell and another T cell, or may be different types of cells such as a T cell and an NK cell.

Whether the antigen-binding molecule of the present invention does “not bind to CD3 and CD137 at the same time” can be confirmed by: confirming the antigen-binding molecule to have binding activity against both CD3 and CD137; then allowing either CD3 or CD137 to bind in advance to the antigen-binding molecule comprising the variable region having this binding activity; and then determining the presence or absence of its binding activity against the other one by the method mentioned above. Alternatively, this can also be confirmed by determining whether the binding of the antigen-binding molecule to either CD3 or CD137 immobilized on an ELISA plate or a sensor chip is inhibited by the addition of the other one into the solution. In some embodiments, the binding of the antigen-binding molecule of the present invention to either CD3 or CD137 is inhibited by binding of the antigen-binding molecule to the other by at least 50%, preferably 60% or more, more preferably 70% or more, more preferably 80% or more, further preferably 90% or more, or even more preferably 95% or more.

In one aspect, while one antigen (e.g. CD3) is immobilized, the inhibition of the binding of the antigen-binding molecule to CD3 can be determined in the presence of the other antigen (e.g. CD137) by methods known in prior art (i.e. ELISA, BIACORE, and so on). In another aspect, while CD137 is immobilized, the inhibition of the binding of the antigen-binding molecule to CD137 also can be determined in the presence of CD3. When either one of two aspects mentioned above is conducted, the antigen-binding molecule of the present invention is determined not to bind to CD3 and CD137 at the same time if the binding is inhibited by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the competition measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

In one aspect, while one antigen (e.g. CD3) is immobilized, the attenuation of the binding signal of the antigen-binding molecule to CD3 can be determined in the presence of the other antigen (e.g. CD137) by methods known in prior art (i.e. ELISA, ECL and so on). In another aspect, while CD137 is immobilized, the attenuation of the binding signal of the antigen-binding molecule to CD137 also can be determined in the presence of CD3. When either one of two aspects mentioned above is conducted, the antigen-binding molecule of the present invention is determined not to bind to CD3 and CD137 at the same time if the binding signal is attenuated by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

Specifically, in the case of using, for example, the ECL method, a biotin-labeled antigen-binding molecule to be tested, CD3 labeled with sulfo-tag (Ru complex), and an unlabeled CD137 are prepared. When the antigen-binding molecule to be tested is capable of binding to CD3 and CD137, but does not bind to CD3 and CD137 at the same time, the luminescence signal of the sulfo-tag is detected in the absence of the unlabeled CD137 by adding the mixture of the antigen-binding molecule to be tested and labeled CD3 onto a streptavidin-immobilized plate, followed by light development. By contrast, the luminescence signal is decreased in the presence of unlabeled CD137. This decrease in luminescence signal can be quantified to determine relative binding activity. This analysis may be similarly conducted using the labeled CD137 and the unlabeled CD3.

In the case of the ALPHAScreen, the antigen-binding molecule to be tested interacts with CD3 in the absence of the competing CD137 to generate signals of 520 to 620 nm. The untagged CD137 competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis. This analysis may be similarly conducted using the tagged CD137 and the untagged CD3.

Alternatively, a method using fluorescence resonance energy transfer (FRET) may be used. FRET is a phenomenon in which excitation energy is transferred directly between two fluorescent molecules located in proximity to each other by electron resonance. When FRET occurs, the excitation energy of a donor (fluorescent molecule having an excited state) is transferred to an acceptor (another fluorescent molecule located near the donor) so that the fluorescence emitted from the donor disappears (to be precise, the lifetime of the fluorescence is shortened) and instead, the fluorescence is emitted from the acceptor. By use of this phenomenon, whether or not bind to CD3 and CD137 at the same time can be analyzed. For example, when CD3 carrying a fluorescence donor and CD137 carrying a fluorescence acceptor bind to the antigen-binding molecule to be tested at the same time, the fluorescence of the donor disappears while the fluorescence is emitted from the acceptor. Therefore, change in fluorescence wavelength is observed. Such an antibody is confirmed to bind to CD3 and CD137 at the same time. On the other hand, if the mixing of CD3, CD137, and the antigen-binding molecule to be tested does not change the fluorescence wavelength of the fluorescence donor bound with CD3, this antigen-binding molecule to be tested can be regarded as antigen binding domain that is capable of binding to CD3 and CD137, but does not bind to CD3 and CD137 at the same time.

For example, a biotin-labeled antigen-binding molecule to be tested is allowed to bind to streptavidin on the donor bead, while CD3 tagged with glutathione S transferase (GST) is allowed to bind to the acceptor bead. The antigen-binding molecule to be tested interacts with CD3 in the absence of the competing second antigen to generate signals of 520 to 620 nm. The untagged second antigen competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis.

The tagging is not limited to the GST tagging and may be carried out with any tag such as, but not limited to, a histidine tag, MBP, CBP, a Flag tag, an HA tag, a V5 tag, or a c-myc tag. The binding of the antigen-binding molecule to be tested to the donor bead is not limited to the binding using biotin-streptavidin reaction. Particularly, when the antigen-binding molecule to be tested comprises Fc, a possible method involves allowing the antigen-binding molecule to be tested to bind via an Fc-recognizing protein such as protein A or protein G on the donor bead.

Also, the case where the variable region is capable of binding to CD3 and CD137 at the same time when CD3 and CD137 are not expressed on cell membranes, as with soluble proteins, or both reside on the same cell, but cannot bind to CD3 and CD137 each expressed on a different cell, at the same time can also be assayed by a method known in the art.

Specifically, the antigen-binding molecule to be tested has been confirmed to be positive in ECL-ELISA for detecting binding to CD3 and CD137 at the same time is also mixed with a cell expressing CD3 and a cell expressing CD137. The antigen-binding molecule to be tested can be shown to be incapable of binding to CD3 and CD137 expressed on different cells, at the same time unless the antigen-binding molecule and these cells bind to each other at the same time. This assay can be conducted by, for example, cell-based ECL-ELISA. The cell expressing CD3 is immobilized onto a plate in advance. After binding of the antigen-binding molecule to be tested thereto, the cell expressing CD137 is added to the plate. A different antigen expressed only on the cell expressing CD137 is detected using a sulfo-tag-labeled antibody against this antigen. A signal is observed when the antigen-binding molecule binds to the two antigens respectively expressed on the two cells, at the same time. No signal is observed when the antigen-binding molecule does not bind to these antigens at the same time.

Alternatively, this assay may be conducted by the ALPHAScreen method. The antigen-binding molecule to be tested is mixed with a cell expressing CD3 bound with the donor bead and a cell expressing CD137 bound with the acceptor bead. A signal is observed when the antigen-binding molecule binds to the two antigens expressed on the two cells respectively, at the same time. No signal is observed when the antigen-binding molecule does not bind to these antigens at the same time.

Alternatively, this assay may also be conducted by an Octet interaction analysis method. First, a cell expressing CD3 tagged with a peptide tag is allowed to bind to a biosensor that recognizes the peptide tag. A cell expressing CD137 and the antigen-binding molecule to be tested are placed in wells and analyzed for interaction. A large wavelength shift caused by the binding of the antigen-binding molecule to be tested and the cell expressing CD137 to the biosensor is observed when the antigen-binding molecule binds to the two antigens expressed on the two cells respectively, at the same time. A small wavelength shift caused by the binding of only the antigen-binding molecule to be tested to the biosensor is observed when the antigen-binding molecule does not bind to these antigens at the same time.

Instead of these methods based on the binding activity, assay based on biological activity may be conducted. For example, a cell expressing CD3 and a cell expressing CD137 are mixed with the antigen-binding molecule to be tested, and cultured. The two antigens expressed on the two cells respectively are mutually activated via the antigen-binding molecule to be tested when the antigen-binding molecule binds to these two antigens at the same time. Therefore, change in activation signal, such as increase in the respective downstream phosphorylation levels of the antigens, can be detected. Alternatively, cytokine production is induced as a result of the activation. Therefore, the amount of cytokines produced can be measured to thereby confirm whether or not to bind to the two cells at the same time. Alternatively, cytotoxicity against a cell expressing CD137 is induced as a result of the activation. Alternatively, the expression of a reporter gene is induced by a promoter which is activated at the downstream of the signal transduction pathway of CD137 or CD3 as a result of the activation. Therefore, the cytotoxicity or the amount of reporter proteins produced can be measured to thereby confirm whether or not to bind to the two cells at the same time.

Fab Molecule

A “Fab molecule” refers to a protein consisting of the VH and CH1 domain of the heavy chain (the “Fab heavy chain”) and the VL and CL domain of the light chain (the “Fab light chain”) of an immunoglobulin.

Fused

By “fused” is meant that the components (e.g. a Fab molecule and an Fc domain subunit) are linked by peptide bonds, either directly or via one or more peptide linkers.

“Crossover” Fab

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

“Conventional” Fab

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called alpha (IgA), delta (IgD), epsilon (IgE), gamma (IgG), or mu (IgM), some of which may be further divided into subtypes, e.g. gamma 1 (IgG1), gamma 2 (IgG2), gamma 3 (IgG3), gamma 4 (IgG4), alpha 1 (IgA1) and alpha 2 (IgA2). The light chain of an immunoglobulin may be assigned to one of two types, called kappa and lambda, based on the amino acid sequence of its constant domain. An immunoglobulin essentially consists of two Fab molecules and an Fc domain, linked via the immunoglobulin hinge region.

Affinity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

Methods to Determine Affinity

In certain embodiments, the antigen-binding molecule or antibody provided herein has a dissociation constant (KD) of 1 micromolar (micro M) or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸ M or less, 10⁻⁸ M to 10⁻¹³ M, 10⁻⁹ M to 10⁻¹¹ M) for its antigen. In certain embodiments, the KD value of the antibody/antigen-binding molecule for CD3, CD137 or DLL3 falls within the range of 1-40, 1-50, 1-70, 1-80, 30-50, 30-70, 30-80, 40-70, 40-80, or 60-80 nM.

In one embodiment, KD is measured by a radiolabeled antigen-binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 microgram/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 microliter/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, N.J.) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 microgram/ml (approx. 0.2 micromolar) before injection at a flow rate of 5 microliter/minute to achieve approximately 10 response units (RU) of coupled protein. Following the injection of antigen, 1 M ethanolamine is injected to block unreacted groups. For kinetics measurements, twofold serial dilutions of Fab (0.78 nM to 500 nM) are injected in PBS with 0.05% polysorbate 20 (TWEEN-20™) surfactant (PBST) at 25 degrees C. at a flow rate of approximately 25 microliter/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to the methods for measuring the affinity of the antigen-binding molecule or the antibody described above, persons skilled in art can carry out affinity measurement for other antigen-binding molecules or antibodies, towards various kind of antigens.

Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, Md., 1991.

Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂(“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

Isolated (Nucleic Acid)

An “isolated” nucleic acid molecule is one which has been separated from a component of its natural environment. An isolated nucleic acid molecule further includes a nucleic acid molecule contained in cells that ordinarily contain the nucleic acid molecule, but the nucleic acid molecule is present extrachromosomally or at a chromosomal location that is different from its natural chromosomal location.

Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

Specificity

“Specific” means that a molecule that binds specifically to one or more binding partners does not show any significant binding to molecules other than the partners. Furthermore, “specific” is also used when an antigen-binding site is specific to a particular epitope of multiple epitopes contained in an antigen. If an antigen-binding molecule binds specifically to an antigen, it is also described as “the antigen-binding molecule has/shows specificity to/towards the antigen”. When an epitope bound by an antigen-binding site is contained in multiple different antigens, an antigen-binding molecule containing the antigen-binding site can bind to various antigens that have the epitope.

Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

Variable Fragment (Fv)

Herein, the term “variable fragment (Fv)” refers to the minimum unit of an antibody-derived antigen-binding site that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)₂

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen-binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is a single chain low molecule weight antibody in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)₂ is a single chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)₂ preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, an sc(Fv)₂ includes two VH units and two VL units which are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.

[VL]-linker-[VH]-linker-[VH]-linker-[VL]

[VH]-linker-[VL]-linker-[VL]-linker-[VH]

[VH]-linker-[VH]-linker-[VL]-linker-[VL]

[VL]-linker-[VL]-linker-[VH]-linker-[VH]

[VL]-linker-[VH]-linker-[VL]-linker-[VH]

The molecular form of sc(Fv)₂ is also described in detail in WO 2006/132352. According to these descriptions, those skilled in the art can appropriately prepare desired sc(Fv)₂ to produce the polypeptide complexes disclosed herein.

Furthermore, the antigen-binding molecules or antibodies of the present disclosure may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present disclosure. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

Ser,
Gly-Ser,
Gly-Gly-Ser,
Ser-Gly-Gly,
(SEQ ID NO: 91)
Gly-Gly-Gly-Ser,
(SEQ ID NO: 92)
Ser-Gly-Gly-Gly,
(SEQ ID NO: 93)
Gly-Gly-Gly-Gly-Ser,
(SEQ ID NO: 94)
Ser-Gly-Gly-Gly-Gly,
(SEQ ID NO: 95)
Gly-Gly-Gly-Gly-Gly-Ser,
(SEQ ID NO: 96)
Ser-Gly-Gly-Gly-Gly-Gly,
(SEQ ID NO: 97)
Gly-Gly-Gly-Gly-Gly-Gly-Ser,
(SEQ ID NO: 98)
Ser-Gly-Gly-Gly-Gly-Gly-Gly,
(Gly-Gly-Gly-Gly-Ser (SEQ ID NO: 93))n,
and
(Ser-Gly-Gly-Gly-Gly (SEQ ID NO: 94))n,

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:

N-hydroxy succinimide (NHS),

disuccinimidyl suberate (DSS),

bis(sulfosuccinimidyl) suberate (BS3),

dithiobis(succinimidyl propionate) (DSP),

dithiobis(sulfosuccinimidyl propionate) (DTSSP),

ethylene glycol bis(succinimidyl succinate) (EGS),

ethylene glycol bis(sulfosuccinimidyl succinate) (sulfo-EGS),

disuccinimidyl tartrate (DST), disulfosuccinimidyl tartrate (sulfo-DST),

bis[2-(succinimidoxycarbonyloxy)ethyl] sulfone (BSOCOES), and

bis[2-(sulfosuccinimidoxycarbonyloxy)ethyl] sulfone (sulfo-BSOCOES). These crosslinking agents are commercially available.

In general, three linkers are required to link four antibody variable regions together. The linkers to be used may be of the same type or different types.

Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding site is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

Fc Region

The term “Fc region” or “Fc domain” refers to a region comprising a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)2 under appropriately set reaction conditions (e.g., pH) of the enzyme. Examples thereof can include pepsin and papain.

An Fc region derived from, for example, naturally occurring IgG can be used as the “Fc region” of the present invention. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present invention. Particularly, the sequence of human IgG1 may have DEL or EEM as an amino acid sequence of EU numbering positions 356 to 358.

In some embodiments, the Fc domain of the multispecific antigen binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the multispecific antigen binding molecule described herein comprises not more than one Fc domain.

In one embodiment described herein the Fc domain of the multispecific antigen binding molecule is an IgG Fc domain. In a particular embodiment the Fc domain is an IgG1 Fc domain. In another embodiment the Fc domain is an IgG1 Fc domain. In a further particular embodiment the Fc domain is a human IgG1 Fc region.

In an embodiment, the multispecific antigen-binding molecule comprises a Fc domain.

In an embodiment, the Fc domain is composed of a first and a second Fc region subunit capable of stable association, and the Fc domain exhibits reduced binding affinity to human Fc gamma receptor, as compared to a native human IgG1 Fc domain.

In an embodiment, the Fc domain exhibits enhanced FcRn-binding activity under an acidic pH condition (e.g., pH 5.8) as compared to that of an Fc region of a native IgG.

In an embodiment, the Fc domain comprises Ala at position 434; Glu, Arg, Ser, or Lys at position 438; and Glu, Asp, or Gln at position 440, according to EU numbering.

In an embodiment, the Fc domain comprises Ala at position 434; Arg or Lys at position 438; and Glu or Asp at position 440, according to EU numbering.

In an embodiment, the Fc domain further comprises Ile or Leu at position 428; and/or Ile, Leu, Val, Thr, or Phe at position 436, according to EU numbering.

In an embodiment, the Fc domain comprises a combination of amino acid substitutions selected from the group consisting of:
(a) N434A/Q438R/S440E;
(b) N434A/Q438R/S440D;
(c) N434A/Q438K/S440E;
(d) N434A/Q438K/S440D;
(e) N434A/Y436T/Q438R/S440E;
(f) N434A/Y436T/Q438R/S440D;
(g) N434A/Y436T/Q438K/S440E;
(h) N434A/Y436T/Q438K/S440D;
(i) N434A/Y436V/Q438R/S440E;
(j) N434A/Y436V/Q438R/S440D;
(k) N434A/Y436V/Q438K/S440E;
(l) N434A/Y436V/Q438K/S440D;
(m) N434A/R435H/F436T/Q438R/S440E;
(n) N434A/R435H/F436T/Q438R/S440D;
(o) N434A/R435H/F436T/Q438K/S440E;
(p) N434A/R435H/F436T/Q438K/S440D;
(q) N434A/R435H/F436V/Q438R/S440E;
(r) N434A/R435H/F436V/Q438R/S440D;
(s) N434A/R435H/F436V/Q438K/S440E;
(t) N434A/R435H/F436V/Q438K/S440D;
(u) M428L/N434A/Q438R/S440E;
(v) M428L/N434A/Q438R/S440D;
(w) M428L/N434A/Q438K/S440E;
(x) M428L/N434A/Q438K/S440D;
(y) M428L/N434A/Y436T/Q438R/S440E;
(z) M428L/N434A/Y436T/Q438R/S440D;
(aa) M428L/N434A/Y436T/Q438K/S440E;
(ab) M428L/N434A/Y436T/Q438K/S440D;
(ac) M428L/N434A/Y436V/Q438R/S440E;
(ad) M428L/N434A/Y436V/Q438R/S440D;
(ae) M428L/N434A/Y436V/Q438K/S440E;
(af) M428L/N434A/Y436V/Q438K/S440D;
(ag) L235R/G236R/S239K/M428L/N434A/Y436T/Q438R/S440E; and
(ah) L235R/G236R/A327G/A330S/P331S/M428L/N434A/Y436T/Q438R/S440E, according to EU numbering.

In an embodiment, the Fc domain comprises a combination of amino acid substitutions of M428L/N434A/Q438R/S440E.

In an embodiment, the Fc domain is an IgG Fc domain, preferably a human IgG Fc domain, more preferably a human IgG1 Fc domain.

In an embodiment, the Fc domain comprises any of:
(a) a first Fc subunit comprises an amino acid sequence shown in SEQ ID NO: 100 and a second Fc subunit comprises an amino acid sequence shown in SEQ ID NO: 111; or
(b) a first Fc subunit comprises an amino acid sequence shown in SEQ ID NO: 99 and a second Fc subunit comprises an amino acid sequence shown in SEQ ID NO: 109.

Fc Region with a Reduced Fc Gamma Receptor-Binding Activity

Herein, “a reduced Fc gamma receptor-binding activity” means, for example, that based on the above-described analysis method the competitive activity of a test antigen-binding molecule or antibody is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control antigen-binding molecule or antibody.

Antigen-binding molecules or antibodies comprising the Fc domain of a monoclonal IgG1, IgG2, IgG3, or IgG4 antibody can be appropriately used as control antigen-binding molecules or antibodies. The Fc domain structures are shown in SEQ ID NOs: 85 (A is added to the N terminus of RefSeq accession number AAC82527.1), 86 (A is added to the N terminus of RefSeq accession number AAB59393.1), 87 (A is added to the N terminus of RefSeq accession number CAA27268.1), and 88 (A is added to the N terminus of RefSeq accession number AAB59394.1). Furthermore, when an antigen-binding molecule or antibody comprising an Fc domain mutant of an antibody of a particular isotype is used as a test substance, the effect of the mutation of the mutant on the Fc gamma receptor-binding activity is assessed using as a control an antigen-binding molecule or antibody comprising an Fc domain of the same isotype. As described above, antigen-binding molecules or antibodies comprising an Fc domain mutant whose Fc gamma receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred antigen-binding molecules or antibodies include those comprising an Fc domain with a mutation (such as substitution) of at least one amino acid selected from the following amino acid positions: 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering), in the amino acids forming the Fc domain of an antibody of a particular isotype. The isotype of antibody from which the Fc domain originates is not particularly limited, and it is possible to use an appropriate Fc domain derived from a monoclonal IgG1, IgG2, IgG3, or IgG4 antibody. It is preferable to use Fc domains derived from IgG1 antibodies.

The preferred antigen-binding molecules or antibodies include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue after the substitution) in the amino acids forming the Fc domain of IgG1 antibody:
(a) L234F, L235E, P331S;
(b) C226S, C229S, P238S;
(c) C226S, C229S; or
(d) C226S, C229S, E233P, L234V, L235A;
as well as those having an Fc domain which has a deletion of the amino acid sequence at positions 231 to 238.

Furthermore, the preferred antigen-binding molecules or antibodies also include those comprising an Fc domain that has any one of the substitutions shown below, whose positions are specified according to EU numbering in the amino acids forming the Fc domain of an IgG2 antibody:
(e) H268Q, V309L, A330S, and P331S;
(f) V234A;
(g) G237A;
(h) V234A and G237A;
(i) A235E and G237A; or
(j) V234A, A235E, and G237A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG3 antibody:
(k) F241A;
(l) D265A; or
(m) V264A. Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG4 antibody:
(n) L235A, G237A, and E318A;
(o) L235E; or
(p) F234A and L235A. Each number represents the position of an amino acid residue in EU numbering; and the one-letter amino acid symbol before the number represents the amino acid residue before substitution, while the one-letter amino acid symbol after the number represents the amino acid residue after the substitution.

The other preferred antigen-binding molecules or antibodies include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred antigen-binding molecules or antibodies also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the antigen-binding molecules or antibodies comprising an Fc domain in which any one or more of the amino acids at positions 234, 235, and 297 are substituted with alanine are particularly preferred.

The preferred antigen-binding molecules or antibodies also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, antigen-binding molecules or antibodies comprising an Fc domain in which an amino acid at position 265 is substituted with alanine are particularly preferred.

Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in RefSeq accession number NM_000566.3 and RefSeq accession number NP_000557.1, respectively; the polynucleotide sequence and amino acid sequence of Fc gamma RIIA are shown in RefSeq accession number BC020823.1 and RefSeq accession number AAH20823.1, respectively; the polynucleotide sequence and amino acid sequence of Fc gamma RIIB are shown in RefSeq accession number BC146678.1 and RefSeq accession number AAI46679.1, respectively; the polynucleotide sequence and amino acid sequence of Fc gamma RIIIA are shown in RefSeq accession number BC033678.1 and RefSeq accession number AAH33678.1, respectively; and the polynucleotide sequence and amino acid sequence of Fc gamma RIIIB are shown in RefSeq accession number BC128562.1 and RefSeq accession number AAI28563.1, respectively. Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

Production and Purification of Multispecific Antibodies

Multispecific antigen binding molecules described herein comprise two different antigen binding moieties (e.g. the “first antigen binding moiety” and the “second antigen binding moiety”), fused to one or the other of the two subunits of the Fc domain, thus the two subunits of the Fc domain are typically comprised in two nonidentical polypeptide chains. Recombinant co-expression of these polypeptides and subsequent dimerization leads to several possible combinations of the two polypeptides. To improve the yield and purity of multispecific antigen binding molecules in recombinant production, it will thus be advantageous to introduce in the Fc domain of the multispecific antigen binding molecule a modification promoting the association of the desired polypeptides.

Accordingly, in particular embodiments the Fc domain of the multispecific antigen binding molecule described herein comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the multispecific antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In other embodiments, other techniques for promoting the association among H chains and between L and H chains having the desired combinations can be applied to the multispecific antigen-binding molecules of the present invention.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

In addition, other known techniques can also be used for formation of multispecific antibodies of the present invention. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antibodies of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing multispecific antibodies composed of two types of light chains and one type of heavy chain as described in WO2012/023053; technologies for producing multispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antibodies.

Alternatively, even when a multispecific antibody of interest cannot be formed efficiently, a multispecific antibody of the present invention can be obtained by separating and purifying the multispecific antibody of interest from the produced antibodies. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present invention. More specifically, the present invention provides Fc regions produced by deleting glycine at position 446 and lysine at position 447 as specified by EU numbering from the amino acid sequences of two polypeptides constituting an Fc region derived from IgG1, IgG2, IgG3, or IgG4.

Multispecific antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the multispecific antigen binding molecule binds. For example, for affinity chromatography purification of multispecific antigen binding molecules of the invention, a matrix with protein A or protein G may be used. Sequential Protein A or G affinity chromatography and size exclusion chromatography can be used to isolate a multispecific antigen binding molecule. The purity of the multispecific antigen binding molecule can be determined by any of a variety of well-known analytical methods including gel electrophoresis, high pressure liquid chromatography, and the like.

All documents cited herein are incorporated herein by reference.

The following are examples of methods and compositions of the present disclosure. It is understood that various other embodiments may be practiced, given the general description provided above.

### EXAMPLES

### [Example 1] Antibody Generation for (1+2) Trivalent Format and (1+2) Dual/LINC Trivalent Format

### 1.1. Generation and Sequence of Trivalent (1+2) Format and Trivalent (1+2) Dual/LINC Format

CD137 receptor clustering is critical for efficient agonistic activity. To improve cytotoxicity, binding number to CD137 molecules is increased through designing a new trivalent antibody format named as DUAL/LINC, 1+2 (FIG. 1(a), Table 2). Specifically, the new antibody format is a trivalent tri-specific antibody with “1+2” format which comprises two monovalent Dual-Fabs each capable of binding to one CD3 or CD137 but not simultaneously (FvB and FvC of FIG. 1, prepared in Reference Example 3) and one monovalent tumor-antigen binding arm (FvA of FIG. 1), wherein one disulfide bond (“LINC”) is introduced/engineered between the two Dual-Fabs by introducing a cysteine substitution e.g. at the 191 position (S191C with Kabat numbering) of the CH1 domain of each of the two Dual-Fabs (FIG. 1a). Without wishing to be bound by a theory, we envisioned that such engineered disulfide bond (“LINC”) would restrict the antigen (CD3 or CD137) binding orientation of the two Dual-Fabs to cis antigen-binding (i.e. binding to two antigens on the same cell) as a result of steric hindrance or shorter distance between the two Dual-Fabs, thereby improving the safety profile of the trispecific Ab by preventing undesirable crosslinking of two CD3/CD137-expressing immune cells mediated by the two Dual-Fabs in an tumor antigen-independent manner (FIG. 2a). Fc region was Fc gamma R silent and deglycosylated. The target antigen of each Fv region and naming rule of each binding domain in the trispecific antibodies are shown in Table 2 a) and the SEQ ID NOs are shown in Table 2 b) and c).

TABLE 2
a) Antibody name and targeted arm

| Antibody Name | Format | Fv A | Linker | Fv B | Fv C |
|---|---|---|---|---|---|
| DLL3-DualAE05/DualAE05-FF091 | (1 + 2)Dual/LINC | Anti-DLL3 | Long | DualAE05 | DualAE05 |
| Ctrl-DualAE05/DualAE05-FF091 | (1 + 2)Dual/LINC | Ctrl | Long | DualAE05 | DualAE05 |
| DLL3-DualAE05/DualAE05-FF102 | (1 + 2)Dual/LINC | Anti-DLL3 | Long | DualAE05 | DualAE05 |
| DLL3-DualAE05/DualAE05-FF110 | (1 + 2)Dual/LINC | Anti-DLL3 | Mid | DualAE05 | DualAE05 |
| DLL3-DualAE05/DualAE05-FF111 | (1 + 2)Dual/LINC | Anti-DLL3 | Short | DualAE05 | DualAE05 |
| DLL3-DualAE05/DualAE05-FF056 | (1 + 2)Dual/LINC | Anti-DLL3 | Long | DualAE05 | DualAE05 |
| DLL3-DualAE15/DualAE15-FF119 | (1 + 2)Dual/LINC | Anti-DLL3 | Long | DualAE15 | DualAE15 |
| Ctrl-DualAE15/DualAE15-FF119 | (1 + 2)Dual/LINC | Ctrl | Long | DualAE15 | DualAE15 |
| DLL3-DualAE15/DualAE15-FF120 | (1 + 2)Dual/LINC | Anti-DLL3 | Long | DualAE15 | DualAE15 |
| DLL3-DualAE15/DualAE15-FF121 | (1 + 2)Dual/LINC | Anti-DLL3 | Mid | DualAE15 | DualAE15 |
| DLL3-DualAE15/DualAE15-FF122 | (1 + 2)Dual/LINC | Anti-DLL3 | Short | DualAE15 | DualAE15 |
| DLL3-DualAE15/DualAE15-FF123 | (1 + 2)Dual/LINC | Anti-DLL3 | Long | DualAE15 | DualAE15 |
| DLL3-DualAE16/DualAE16-FF124 | (1 + 2)Dual/LINC | Anti-DLL3 | Long | DualAE16 | DualAE16 |
| Ctrl-DualAE16/DualAE16-FF124 | (1 + 2)Dual/LINC | Ctrl | Long | DualAE16 | DualAE16 |
| DLL3-DualAE16/DualAE16-FF125 | (1 + 2)Dual/LINC | Anti-DLL3 | Long | DualAE16 | DualAE16 |
| DLL3-DualAE16/DualAE16-FF126 | (1 + 2)Dual/LINC | Anti-DLL3 | Mid | DualAE16 | DualAE16 |
| DLL3-DualAE16/DualAE16-FF127 | (1 + 2)Dual/LINC | Anti-DLL3 | Short | DualAE16 | DualAE16 |
| DLL3-DualAE16/DualAE16-FF128 | (1 + 2)Dual/LINC | Anti-DLL3 | Long | DualAE16 | DualAE16 |
| GPC3-DualAE05/DualAE05-FF056 | (1 + 2)Dual/LINC | Anti-GPC3 | Long | DualAE05 | DualAE05 |
| DLL3-DualAE05/DualAE05-FF029 | (1 + 2)Dual/LINC | Anti-DLL3 | Long | DualAE05 | DualAE05 |
| Ctrl-DualAE05/DualAE05-FF030 | (1 + 2) No LINC | Ctrl | Long | DualAE05 | DualAE05 |
| GPC3-DualAE05/DualAE05-FF028 | (1 + 2)Dual/LINC | Anti-GPC3 | Long | DualAE05 | DualAE05 |
| DLL3-DualAE05/DualAE05-FF117 | (1 + 2) No LINC | Anti-DLL3 | Long | DualAE05 | DualAE05 |
| DLL3-DualAE05/DualAE05-FF115 | (1 + 2) No LINC | Anti-DLL3 | Mid | DualAE05 | DualAE05 |
| GPC3-DualAE05/DualAE05-FF030 | (1 + 2) No LINC | Anti-GPC3 | Long | DualAE05 | DualAE05 |

b) Antibody chain number and sequence ID

| Variant name | Linker | Chain 1 | Chain 2 | Chain 3 | Chain 4&5 |
|---|---|---|---|---|---|
| DLL3-DualAE05/DualAF05-FF091 | 249 | 201 | 206 | 208 | 214 |
| Ctrl-DualAE05/DualAE05-FF091 | 249 | 202 | 207 | 208 | 214 |
| DLL3-DualAE05/DualAE05-FF102 | 249 | 203 | 206 | 209 | 214 |
| DLL3-DualAE05/DualAE05-FF110 | 248 | 204 | 206 | 209 | 214 |
| DLL3 DualAE05/DualAE05-FF111 | 259 | 205 | 206 | 209 | 214 |
| DLL3-DualAE05/DualAE05-FF056 | 249 | 216 | 206 | 225 | 214 |
| DLL3-DualAE15/DualAE15-FF119 | 249 | 217 | 206 | 210 | 214 |
| Ctrl-DualAE15/DualAE15-FF119 | 249 | 218 | 207 | 210 | 214 |
| DLL3-DualAE15/DualAE15-FF120 | 249 | 219 | 206 | 211 | 214 |
| DLL3-DualAE15/DualAE15-FF121 | 248 | 220 | 206 | 211 | 214 |
| DLL3-DualAE15/DualAE15-FF122 | 259 | 221 | 206 | 211 | 214 |
| DLL3-DualAE15/DualAE15-FF123 | 249 | 222 | 206 | 230 | 214 |
| DLL3-DualAE16/DualAE15-FF124 | 249 | 223 | 206 | 212 | 215 |
| Ctrl-DualAE16/DualAE16-FF124 | 249 | 224 | 207 | 212 | 215 |
| DLL3-DualAE16/DualAE16-FF125 | 249 | 225 | 206 | 213 | 215 |
| DLL3-DualAE16/DualAF16-FF126 | 248 | 226 | 206 | 213 | 215 |
| DLL3-DualAE16/DualAE16-FF127 | 259 | 227 | 206 | 213 | 215 |
| DLL3-DualAE16/DualAE16-FF128 | 249 | 228 | 206 | 231 | 215 |
| GPC3-DualAE05/DualAE05-FF056 | 249 | 321 | 327 | 229 | 214 |
| DLL3-DualAE05/DualAE05-FF029 | 249 | 322 | 206 | 328 | 214 |
| Ctrl-DualAE05/DualAE05-FF030 | 249 | 323 | 207 | 329 | 214 |
| GPC3-DualAE05/DualAE05-FF028 | 249 | 324 | 327 | 229 | 214 |
| DLL3-DualAE05/DualAE05-FF117 | 249 | 325 | 206 | 330 | 214 |
| DLL3-DualAE05/DualAE05-FF115 | 248 | 326 | 206 | 330 | 214 |
| GPC3-DualAE05/DualAE05-FF030 | 249 | 339 | 327 | 329 | 214 |

c) Sequence ID of variable region and their CDR1 to CDR3

| VR name | VHR name | VLR name | VHR | VHR_CDR1 | VHR_CDR2 | VHR_CDR3 |
|---|---|---|---|---|---|---|
| DLL3 | D08410053H0118 | D084101L0000 | 232 | 233 | 234 | 235 |
| DualAE05 | dBBDu183H1643 | dBBDu072L0581 | 6 | 20 | 34 | 48 |
| DualAE15 | dBBDu183H2594 | dBBDu072L0581 | 14 | 28 | 42 | 56 |
| DualAE16 | dBBDu183H1644 | dBBDu072L0939 | 81 | 82 | 83 | 84 |
| CD3ε | CD3εVH | CD3εVL | 251 | 252 | 253 | 254 |
| Ctrl | IC17HdK | IC17L | 240 | 241 | 242 | 243 |
| GPC3 | GCH065H | TR01L0011 | 331 | 332 | 333 | 334 |

c) Sequence ID of variable region and their CDR1 to CDR3

| VR name | VLR | VLR_CDR1 | VLR_CDR2 | VLR_CDR3 |
|---|---|---|---|---|
| DLL3 | 236 | 237 | 238 | 239 |
| DualAE05 | 58 | 63 | 68 | 73 |
| DualAE15 | 58 | 63 | 68 | 73 |
| DualAE16 | 60 | 65 | 70 | 75 |
| CD3ε | 255 | 256 | 257 | 258 |
| Ctrl | 244 | 245 | 246 | 247 |
| GPC3 | 335 | 336 | 337 | 338 |

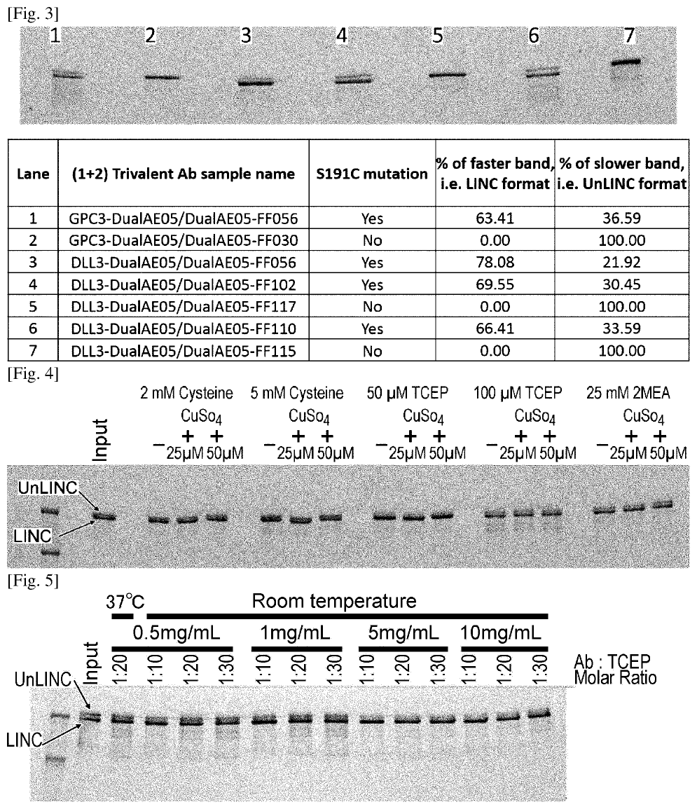

All antibodies were expressed as trivalent form by transient expression in Expi293 cells (Invitrogen) and purified according to Reference EXAMPLE 1. Purities of antibodies were analysed by non-reducing SDS-PAGE (Reference EXAMPLE 2) as showed in FIG. 3, double bands were observed for the (1+2) Dual/LINC trivalent antibody samples. It has been shown that protein tertiary structure may affect polypeptide SDS-PAGE migration rates in which a disulfide linked conformation caused an increased in SDS-PAGE migration rate in non-reducing condition (Therien A G, Grant F E, Deber C M (2001) Interhelical hydrogen bonds in the CFTR membrane domain. Nat Struct Biol 8:597-601.). In FIG. 3, a single protein migration band for the (1+2) trivalent format without introduction of the S191C mutations (FIG. 3, lanes 2, 5 and 7) was observed. Whereas two protein migration bands were detected for the (1+2) Dual/LINC antibody variants sample with the S191C mutation, the slower migration band (or upper band) showed similar electrophoretic mobility as the (1+2) trivalent format without introduction of the S191C mutations. This suggests that the faster migration band (or lower band) was the trivalent 1+2 antibody with the engineered disulfide bond (Dual/LINC, FIG. 1a). As the UnLINC format (i.e., trivalent 1+2 antibody without the engineered disulfide bond, FIG. 1b) might resulted in crosslinking of CD137 and/or CD3-expressing immune cells in the absence of binding to tumor antigen (as depicted in FIG. 2a), further purification to reduce UnLINC format in the final format product is required.

### [Example 2] Dual/LINC Purity Improvement by Reducing Reagent Treatment

### Example 2.1 Promotion of “Paired Cysteines” (Engineered Disulfide Bond) Formation in Dual/LINC Antibody Using Reducing Agents

While not wishing to be bound by the following theory, it is believed that the presence of UnLINC format (i.e., trivalent 1+2 antibody without the engineered disulfide bond or “unpaired cysteines” form) could be due to the unpaired Cys residues often form disulfide bonds with molecule that contains free thiol group, such as cysteinylation and glutathionylation which “capped” the unpaired Cys residues and prevents LINC formation (formation of engineered disulfide bond). As shown in FIG. 2(b), to remove the capped molecules of unpair cysteines, reducing agents can help de-cap the surface cysteines and further re-oxidation (e.g. remove reducing reagent via buffer exchange) of de-capped antibody can promote disulfide bond formation between the de-capped cysteines for LINC formation. Hence, removal of cysteinylation from the unpaired sulfhydryl in the UnLINC format via reduction and re-oxidation could remove the UnLINC format and improves homogeneity of the antibodies.

To obtain homogenous Dual-LINC antibody preparation, the unpaired cysteines which were capped with cysteine needed to be de-capped to promote “paired cysteines” (engineered disulfide bond) formation. To de-cap the unpaired cysteines, various reducing agents were used. The heterogeneous Dual-LINC antibody preparation comprising paired and unpaired cysteines forms were subjected to decapping, by the addition of reducing agents such as Cysteine or TCEP (tris (2-carboxyethyl) phosphine) or 2MEA (2-Mercaptoethylamine). De-capping was followed by buffer exchange to remove the reducing agents and promote “paired cysteines” (engineered disulfide bond) formation in Dual-LINC antibody preparation. Apart from buffer exchange, CuSO₄ which is known to promote cysteine bond formation, was used to enhance “paired cysteines” (engineered disulfide bond) formation. 0.5 mg/ml (2.5 micromolar) of Dual-LINC antibody preparation was treated with 2 mM cysteine, 5 mM cysteine, 50 micromolar TCEP, 100 micromolar TCEP and 25 mM 2MEA at 37 degrees C. for 2 hours followed by buffer exchange with 1×TBS either without CuSO₄ or with 25 micromolar/50 micromolar CuSO₄. Of all the conditions, it was observed that TCEP treatment showed significant increase in “paired cysteines” (engineered disulfide bond) formation in Dual-LINC antibody preparation. Cysteine, 2MEA and CuSO₄ addition did not increase the “paired cysteines” formation (engineered disulfide bond) like TCEP (FIG. 4).

To further optimize TCEP treatment, various concentrations of Dual-LINC-Ig were incubated with different molar ratios of TCEP at room temperature for 2 hours followed by buffer exchange to 1×PBS (to remove TCEP) to promote “paired cysteines” formation. Dual-LINC antibody preparation and TCEP were added in 1:10, 1:20 and 1:30 molar ratios at different concentrations of Dual-LINC-Ig at 0.5 mg/ml (2.5 micromolar), 1 mg/ml (5 micromolar), 5 mg/ml (25 micromolar) and 10 mg/ml (50 micromolar). High concentrations such as 5 mg/ml and 10 mg/ml have also shown significant increase in “paired cysteines” (engineered disulfide bond) formation based on SDS-PAGE analysis (FIG. 5).

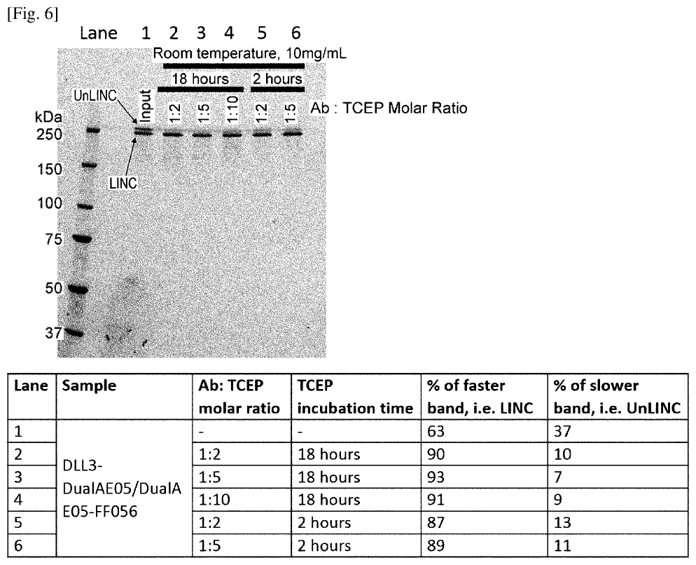

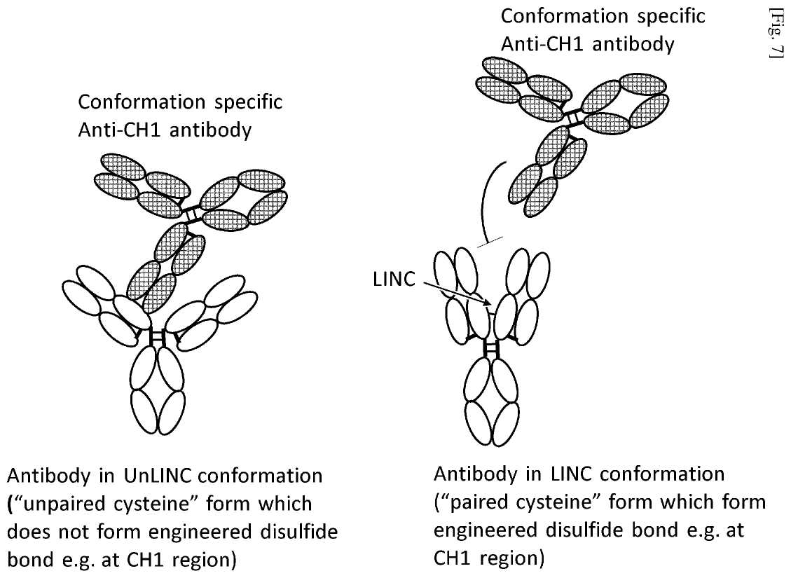

To further optimize the incubation period for TCEP treatment, the reaction was performed with different incubation periods of 2 hours or 18 hours with 1:2, 1:5 and 1:10 molar ratios of Dual-LINC-Ig (50 micromolar) and TCEP. In all the conditions, Dual-LINC-Ig with unpaired cysteines (engineered disulfide bond) reduced to <10% based on SDS-PAGE analysis (intensity of slower band/upper band corresponding to “UnLINC” format divided by the intensity sum of two bands correspond to “LINC” and “UnLINC” structure in FIG. 6) and further increased the homogeneity of Dual-LINC-Ig (FIG. 6). Of all the conditions, 1:5 ratio of Dual-LINC antibody preparation and TCEP with 18-hr incubation period at RT followed by buffer exchange to 1×PBS for overnight (O/N) re-oxidation showed the best Dual-LINC with paired cysteines formation.

The following shows examples of amino acid sequences of the present invention.

TABLE 3

| SEQ | |
|---|---|
| number | Amino Acid Sequence |
| 201 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQEGNVFSCSVLHEALHAHYTRKELSLSP |
| 202 | DIQMTQSSSSFSVSLGDRVTITCKASEDIYNRLAWYQQKPGNAPRLLISGATSLETGVPSRFSGSGSGKDYTLSITSLQTEDVATYYC |
| | QQYWSTPYTFGGGTKLEVKSSASTKGPSVFPLAPSSRSTSESTAALGCLVKDYFPEPVTVSWNSGALTSGVHTFPAVLQSSGLYSLSS |
| | VVTVPSSSLGTKTYTCNVDHKPSNTKVDKRVEPKSCGGGGSGGGGSQVQLVESGGGLVQPGRSLRLSCAASGFKFSNVWFHWVRQAPG |
| | KGLEWVAQIKDYYNAYAAYYAPSVKGRFTISRDDSKNSIYLQMNSLKTEDTAVYYCHYVHYASASTLLPAEGVDAWGQGTTVTVSSAS |
| | TKGPSVFPLAPSSKSTSGGTAALGCLVEDYFPEPVTVSWNSGALTSGVHTFPAVLQSSGLYSLSSVVTVPSCSLGTQTYICNVNHKPS |
| | NTKVDEKVEPKSCDKTHTCPPCPAPEAAGGPSVFLFPPKPKDTLMISRTPEVTCVVVDVSHEDPEVKFNWYVDGVEVHNAKTKPREEQ |
| | YASTYRVVSVLTVLHQDWLNGKEYKCKVSNKALPAPIEKTISKAKGQPREPQVYTLPPCRDELTKNQVSLWCLVKGFYPSDIAVEWES |
| | NGQPENNYKTTPPVLDSDGSFFLYSKLTVDKSRWQEGNVFSCSVLHEALHAHYTRKELSLSP |
| 203 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQQGNVFSCSVLHEALHAHYTRKELSLSP |
| 204 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSQVQLVESGGGLVQPGRSLRLSCAASGFKFSNVWFHWVRQAPGKG |
| | LEWVAQIKDYYNAYAAYYAPSVKGRFTISRDDSKNSIYLQMNSLKTEDTAVYYCHYVHYASASTLLPAEGVDAWGQGTTVTVSSASTK |
| | GPSVFPLAPSSKSTSGGTAALGCLVEDYFPEPVTVSWNSGALTSGVHTFPAVLQSSGLYSLSSVVTVPSCSLGTQTYICNVNHKPSNT |
| | KVDEKVEPKSCDKTHTCPPCPAPEAAGGPSVFLFPPKPKDTLMISRTPEVTCVVVDVSHEDPEVKFNWYVDGVEVHNAKTKPREEQYA |
| | STYRVVSVLTVLHQDWLNGKEYKCKVSNKALPAPIEKTISKAKGQPREPQVYTLPPCRDELTKNQVSLWCLVKGFYPSDIAVEWESNG |
| | QPENNYKTTPPVLDSDGSFFLYSKLTVDKSRWQQGNVFSCSVLHEALHAHYTRKELSLSP |
| 205 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCQVQLVESGGGLVQPGRSLRLSCAASGFKFSNVWFHWVRQAPGKGLEWVA |
| | QIKDYYNAYAAYYAPSVKGRFTISRDDSKNSIYLQMNSLKTEDTAVYYCHYVHYASASTLLPAEGVDAWGQGTTVTVSSASTKGPSVF |
| | PLAPSSKSTSGGTAALGCLVEDYFPEPVTVSWNSGALTSGVHTFPAVLQSSGLYSLSSVVTVPSCSLGTQTYICNVNHKPSNTKVDEK |
| | VEPKSCDKTHTCPPCPAPEAAGGPSVFLFPPKPKDTLMISRTPEVTCVVVDVSHEDPEVKFNWYVDGVEVHNAKTKPREEQYASTYRV |
| | VSVLTVLHQDWLNGKEYKCKVSNKALPAPIEKTISKAKGQPREPQVYTLPPCRDELTKNQVSLWCLVKGFYPSDIAVEWESNGQPENN |
| | YKTTPPVLDSDGSFFLYSKLTVDKSRWQQGNVFSCSVLHEALHAHYTRKELSLSP |
| 206 | QVTLRESGPALVKPTQTLTLTCTFSGFSLSSSYDMGWVRQAPGQGLEWMGTIYTGDYSTDYASWAKGRVTISVDRSKNQFSLKLSSVT |
| | AADTAVYYCARHTGYGYFGLWGQGTLVTVSSASVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 207 | QVQLQQSGPQLVRPGASVKISCKASGYSFTSYWMHWVNQRPGQGLEWIGMIDPSYSETRLNQKFKDKATLTVDKSSSTAYMQLSSPTS |
| | EDSAVYYCALYGNYFDYWGQGTTLTVSSASVAAPSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| 208 | QVQLVESGGGLVQPGRSLRLSCAASGFKFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPEAAGGPSVFLFPPKPKDTLMI |
| | SRTPEVTCVVVDVSHEDPEVKFNWYVDGVEVHNAKTKPREEQYASTYRWSVLTVLHQDWLNGKEYKCKVSNKALPAPIEKTISKAKGQ |
| | PREPQVCTLPPSRDELTKNQVSLSCAVKGFYPSDIAVEWESNGQPENNYKTTPPVLDSDGSFFLVSKLTVDKSRWQEGNVFSCSVLHE |
| | ALHAHYTRKELSLSP |
| 209 | QVQLVESGGGLVQPGRSLRLSCAASGFKFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGFQTYICNVNHKPSNTKVDEKVEPKSCDKTHTCPPCPAPEAAGGPSVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSRDELTKNQVSLSCAVKGFYPSDIAVEWESNGQPENNYKTTPPVLDSDGSFFLVSKLTVDKSRWQQGNVFSCSVLH |
| | EALHAHYTRKELSLSP |
| 210 | QVQLVESGGGLVQPGRSLRLSCAASGFKFSNVWFHWVRQAPGKGLEWVAQIKDYYNAYAGYYHPSVKGRFTISRDDSKNSIYLQMNSL |
| | KTEDTAVYYCHYVHYAAASQLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPEAAGGPSVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSRDELTKNQVSLSCAVKGFYPSDIAVEWESNGQPENNYKTTPPVLDSDGSFFLVSKLTVDKSRWQEGNVFSCSVLH |
| | EALHAHYTRKELSLSP |
| 211 | QVQLVESGGGLVQPGRSLRLSCAASGFKFSNVWFHWVRQAPGKGLEWVAQIKDYYNAYAGYYHPSVKGRFTISRDDSKNSIYLQMNSL |
| | KTEDTAVYYCHYVHYAAASQLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPEAAGGPSVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSRDELTKNQVSLSCAVKGFYPSDIAVEWESNGQPENNYKTTPPVLDSDGSFFLVSKLTVDKSRWQQGNVFSCSVLH |
| | EALHAHYTRKELSLSP |
| 212 | QVQLVESGGGLVQPGRSLRLSCAASGFVFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPEAAGGPSVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSRDELTKNQVSLSCAVKGFYPSDIAVEWESNGQPENNYKTTPPVLDSDGSFFLVSKLTVDKSRWQEGNVFSCSVLH |
| | EALHAHYTRKELSLSP |
| 213 | QVQLVESGGGLVQPGRSLRLSCAASGFVFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPEAAGGPSVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSRDELTKNQVSLSCAVKGFYPSDIAVEWESNGQPENNYKTTPPVLDSDGSFFLVSKLTVDKSRWQQGNVFSCSVLH |
| | EALHAHYTRKELSLSP |
| 214 | DIVMTQSPLSLPVTPGEPASISCQPSQEVVHMNRNTYLHWYQQKPGQAPRLLIYKVSNRFPGVPDRFSGSGSGTDFTLKISRVEAEDV |
| | GVYYCAQGTSHPFTFGQGTKLEIKRTVAAPSVFIFPPSDRKLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 215 | DIVMTQSPLSLPVTPGEPASISCQPSQEVVHMNRNTYLHWYQQKPGQAPRLLIYKVSNVFPGVPDRFSGSGSGTDFTLKISRVEAEDV |
| | GVYYCAQGTHHPFTFGQGTKLEIKRTVAAPSVFIFPPSDRKLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 216 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPELRGGPKVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCREEMTKNQVSLWCLVKGFYPSDIAVE |
| | WESNGQPENNYKTTPPVLDSDGSFFLYSKLTVDKSRWQEGNVFSCSVMHEALHNHYTQKSLSLSP |
| 217 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAGYYHPSVKGRFTISRDDSKNSIYLQMNSLKTEDTAVYYCHYVHYAAASQLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQEGNVFSCSVLHEALHAHYTRKELSLSP |
| 218 | DIQMTQSSSSFSVSLGDRVTITCKASEDIYNRLAWYQQKPGNAPRLLISGATSLETGVPSRFSGSGSGKDYTLSITSLQTEDVATYYC |
| | QQYWSTPYTFGGGTKLEVKSSASTKGPSVFPLAPSSRSTSESTAALGCLVKDYFPEPVTVSWNSGALTSGVHTFPAVLQSSGLYSLSS |
| | VVTVPSSSLGTKTYTCNVDHKPSNTKVDKRVEPKSCGGGGSGGGGSQVQLVESGGGLVQPGRSLRLSCAASGFKFSNVWFHWVRQAPG |
| | KGLEWVAQIKDYYNAYAGYYHPSVKGRFTISRDDSKNSIYLQMNSLKTEDTAVYYCHYVHYAAASQLLPAEGVDAWGQGTTVTVSSAS |
| | TKGPSVFPLAPSSKSTSGGTAALGCLVEDYFPEPVTVSWNSGALTSGVHTFPAVLQSSGLYSLSSVVTVPSCSLGTQTYICNVNHKPS |
| | NTKVDEKVEPKSCDKTHTCPPCPAPEAAGGPSVFLFPPKPKDTLMISRTPEVTCVVVDVSHEDPEVKFNWYVDGVEVHNAKTKPREEQ |
| | YASTYRVVSVLTVLHQDWLNGKEYKCKVSNKALPAPIEKTISKAKGQPREPQVYTLPPCRDELTKNQVSLWCLVKGFYPSDIAVEWES |
| | NGQPENNYKTTPPVLDSDGSFFLYSKLTVDKSRWQEGNVFSCSVLHEALHAHYTRKELSLSP |
| 219 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAGYYHPSVKGRFTISRDDSKNSIYLQMNSLKTEDTAVYYCHYVHYAAASQLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQQGNVFSCSVLHEALHAHYTRKELSLSP |
| 220 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSQVQLVESGGGLVQPGRSLRLSCAASGFKFSNVWFHWVRQAPGKG |
| | LEWVAQIKDYYNAYAGYYHPSVKGRFTISRDDSKNSIYLQMNSLKTEDTAVYYCHYVHYAAASQLLPAEGVDAWGQGTTVTVSSASTK |
| | GPSVFPLAPSSKSTSGGTAALGCLVEDYFPEPVTVSWNSGALTSGVHTFPAVLQSSGLYSLSSVVTVPSCSLGTQTYICNVNHKPSNT |
| | KVDEKVEPKSCDKTHTCPPCPAPEAAGGPSVFLFPPKPKDTLMISRTPEVTCVVVDVSHEDPEVKFNWYVDGVEVHNAKTKPREEQYA |
| | STYRVVSVLTVLHQDWLNGKEYKCKVSNKALPAPIEKTISKAKGQPREPQVYTLPPCRDELTKNQVSLWCLVKGFYPSDIAVEWESNG |
| | QPENNYKTTPPVLDSDGSFFLYSKLTVDKSRWQQGNVFSCSVLHEALHAHYTRKELSLSP |
| 221 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCQVQLVESGGGLVQPGRSLRLSCAASGFKFSNVWFHWVRQAPGKGLEWVA |
| | QIKDYYNAYAGYYHPSVKGRFTISRDDSKNSIYLQMNSLKTEDTAVYYCHYVHYAAASQLLPAEGVDAWGQGTTVTVSSASTKGPSVF |
| | PLAPSSKSTSGGTAALGCLVEDYFPEPVTVSWNSGALTSGVHTFPAVLQSSGLYSLSSVVTVPSCSLGTQTYICNVNHKPSNTKVDEK |
| | VEPKSCDKTHTCPPCPAPEAAGGPSVFLFPPKPKDTLMISRTPEVTCVWDVSHEDPEVKFNWYVDGVEVHNAKTKPREEQYASTYRWS |
| | VLTVLHQDWLNGKEYKCKVSNKALPAPIEKTISKAKGQPREPQVYTLPPCRDELTKNQVSLWCLVKGFYPSDIAVEWESNGQPENNYK |
| | TTPPVLDSDGSFFLYSKLTVDKSRWQQGNVFSCSVLHEALHAHYTRKELSLSP |
| 222 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAGYYHPSVKGRFTISRDDSKNSIYLQMNSLKTEDTAVYYCHYVHYAAASQLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPELRGGPKVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCREEMTKNQVSLWCLVKGFYPSDIAVE |
| | WESNGQPENNYKTTPPVLDSDGSFFLYSKLTVDKSRWQEGNVFSCSVMHEALHNHYTQKSLSLSP |
| 223 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFVFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQEGNVFSCSVLHEALHAHYTRKELSLSP |
| 224 | DIQMTQSSSSFSVSLGDRVTITCKASEDIYNRLAWYQQKPGNAPRLLISGATSLETGVPSRFSGSGSGKDYTLSITSLQTEDVATYYC |
| | QQYWSTPYTFGGGTKLEVKSSASTKGPSVFPLAPSSRSTSESTAALGCLVKDYFPEPVTVSWNSGALTSGVHTFPAVLQSSGLYSLSS |
| | VVTVPSSSLGTKTYTCNVDHKPSNTKVDKRVEPKSCGGGGSGGGGSQVQLVESGGGLVQPGRSLRLSCAASGFVFSNVWFHWVRQAPG |
| | KGLEWVAQIKDYYNAYAAYYAPSVKGRFTISRDDSKNSIYLQMNSLKTEDTAVYYCHYVHYASASTLLPAEGVDAWGQGTTVTVSSAS |
| | TKGPSVFPLAPSSKSTSGGTAALGCLVEDYFPEPVTVSWNSGALTSGVHTFPAVLQSSGLYSLSSVVTVPSCSLGTQTYICNVNHKPS |
| | NTKVDEKVEPKSCDKTHTCPPCPAPEAAGGPSVFLFPPKPKDTLMISRTPEVTCVVVDVSHEDPEVKFNWYVDGVEVHNAKTKPREEQ |
| | YASTYRVVSVLTVLHQDWLNGKEYKCKVSNKALPAPIEKTISKAKGQPREPQVYTLPPCRDELTKNQVSLWCLVKGFYPSDIAVEWES |
| | NGQPENNYKTTPPVLDSDGSFFLYSKLTVDKSRWQEGNVFSCSVLHEALHAHYTRKELSLSP |
| 225 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFVFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQQGNVFSCSVLHEALHAHYTRKELSLSP |
| 226 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSQVQLVESGGGLVQPGRSLRLSCAASGFVFSNVWFHWVRQAPGKG |
| | LEWVAQIKDYYNAYAAYYAPSVKGRFTISRDDSKNSIYLQMNSLKTEDTAVYYCHYVHYASASTLLPAEGVDAWGQGTTVTVSSASTK |
| | GPSVFPLAPSSKSTSGGTAALGCLVEDYFPEPVTVSWNSGALTSGVHTFPAVLQSSGLYSLSSVVTVPSCSLGTQTYICNVNHKPSNT |
| | KVDEKVEPKSCDKTHTCPPCPAPEAAGGPSVFLFPPKPKDTLMISRTPEVTCVVVDVSHEDPEVKFNWYVDGVEVHNAKTKPREEQYA |
| | STYRVVSVLTVLHQDWLNGKEYKCKVSNKALPAPIEKTISKAKGQPREPQVYTLPPCRDELTKNQVSLWCLVKGFYPSDIAVEWESNG |
| | QPENNYKTTPPVLDSDGSFFLYSKLTVDKSRWQQGNVFSCSVLHEALHAHYTRKELSLSP |
| 227 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCQVQLVESGGGLVQPGRSLRLSCAASGFVFSNVWFHWVRQAPGKGLEWVA |
| | QIKDYYNAYAAYYAPSVKGRFTISRDDSKNSIYLQMNSLKTEDTAVYYCHYVHYASASTLLPAEGVDAWGQGTTVTVSSASTKGPSVF |
| | PLAPSSKSTSGGTAALGCLVEDYFPEPVTVSWNSGALTSGVHTFPAVLQSSGLYSLSSVVTVPSCSLGTQTYICNVNHKPSNTKVDEK |
| | VEPKSCDKTHTCPPCPAPEAAGGPSVFLFPPKPKDTLMISRTPEVTCVVVDVSHEDPEVKFNWYVDGVEVHNAKTKPREEQYASTYRV |
| | VSVLTVLHQDWLNGKEYKCKVSNKALPAPIEKTISKAKGQPREPQVYTLPPCRDELTKNQVSLWCLVKGFYPSDIAVEWESNGQPENN |
| | YKTTPPVLDSDGSFFLYSKLTVDKSRWQQGNVFSCSVLHEALHAHYTRKELSLSP |
| 228 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFVFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPELRGGPKVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCREEMTKNQVSLWCLVKGFYPSDIAVE |
| | WESNGQPENNYKTTPPVLDSDGSFFLYSKLTVDKSRWQEGNVFSCSVMHEALHNHYTQKSLSLSP |
| 229 | QVQLVESGGGLVQPGRSLRLSCAASGFKFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPELRGGPKVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSREEMTKNQVSLSCAVKGFYPSDIAVEWESNGQPENNYKTTPPVLDSDGSFFLVSKLTVDKSRWQEGNVFSCSVMH |
| | EALHNHYTQKSLSLSP |
| 230 | QVQLVESGGGLVQPGRSLRLSCAASGFKFSNVWFHWVRQAPGKGLEWVAQIKDYYNAYAGYYHPSVKGRFTISRDDSKNSIYLQMNSL |
| | KTEDTAVYYCHYVHYAAASQLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPELRGGPKVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSREEMTKNQVSLSCAVKGFYPSDIAVEWESNGQPENNYKTTPPVLDSDGSFFLVSKLTVDKSRWQEGNVFSCSVMH |
| | EALHNHYTQKSLSLSP |
| 231 | QVQLVESGGGLVQPGRSLRLSCAASGFVFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGTQTYICNVNHKPSNTKVDEKVEPKSCDKTHTCPPCPAPELRGGPKVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSREEMTKNQVSLSCAVKGFYPSDIAVEWESNGQPENNYKTTPPVLDSDGSFFLVSKLTVDKSRWQEGNVFSCSVMH |
| | EALHNHYTQKSLSLSP |
| 232 | QVTLRESGPALVKPTQTLTLTCTFSGFSLSSSYDMGWVRQAPGQGLEWMGTIYTGDYSTDYASWAKGRVTISVDRSKNQFSLKLSSVT |
| | AADTAVYYCARHTGYGYFGLWGQGTLVTVSS |
| 233 | SSYDMG |
| 234 | FIYTGDYSTDYASWAKG |
| 235 | HTGYGYFGL |
| 236 | DIQLTQSPSFLSASVGDRVTITCQSTESVYGSDWLSWYQQKPGQPPKLLIYQASNLEIGVPSRFSGSGSGTDFTLTINSLEAEDAAT |
| | YYCQGYYSGYIYAFGGGTKVEIK |
| 237 | QSTESVYGSDWLS |
| 238 | QASNLEI |
| 239 | QGYYSGYIYA |
| 240 | QVQLQQSGPQLVRPGASVKISCKASGYSFTSYWMHWVNQRPGQGLEWIGMIDPSYSETRLNQKFKDKATLTVDKSSSTAYMQLSSPTS |
| | EDSAVYYCALYGNYFDYWGQGTTLTVSS |
| 241 | SYWMH |
| 242 | MIDPSYSETRLNQKFKD |
| 243 | YGNYFDY |
| 244 | DIQMTQSSSSFSVSLGDRVTITCKASEDIYNRLAWYQQKPGNAPRLLISGATSLETGVPSRFSGSGSGKDYTLSITSLQTEDVATYYC |
| | QQYWSTPYTFGGGTKLEVK |
| 245 | KASEDIYNRLA |
| 246 | GATSLET |
| 247 | QQYWSTPYT |
| 248 | VEPKSCGGGGS |
| 249 | VEPKSCGGGGSGGGGS |
| 250 | QVQLQESGPGLVKPSETLSLTCTVSGGSISSYYWSWIRQPPGKGLEWIGYVYYSGTTNYNPSLKSRVTISVDTSKNQFSLKLSSVTAA |
| | DTAVYYCASIAVTGFYFDYWGQGTLVTVSSGGGGSGGGGSGGGGSEIVLTQSPGTLSLSPGERVTLSCRASQRVNNNYLAWYQQRPGQ |
| | APRLLIYGASSRATGIPDRFSGSGSGTDFTLTISRLEPEDFAVYYCQQYDRSPLTFGGGTKLEIKSGGGGSEVQLVESGGGLVQPGGS |
| | LKLSCAASGFTFNKYAMNWVRQAPGKGLEWVARIRSKYNNYATYYADSVKDRFTISRDDSKNTAYLQMNNLKTEDTAVYYCVRHGNFG |
| | NSYISYWAYWGQGTLVTVSSGGGGSGGGGSGGGGSQTVVTQEPSLTVSPGGTVTLTCGSSTGAVTSGNYPNWVQQKPGQAPRGLIGGT |
| | KFLAPGTPARFSGSLLGGKAALTLSGVQPEDEAEYYCVLWYSNRWVFGGGTKLTVLGGGGDKTHTCPPCPAPELLGGPSVFLFPPKPK |
| | DTLMISRTPEVTCVVVDVSHEDPEVKFNWYVDGVEVHNAKTKPCEEQYGSTYRCVSVLTVLHQDWLNGKEYKCKVSNKALPAPIEKTI |
| | SKAKGQPREPQVYTLPPSREEMTKNQVSLTCLVKGFYPSDIAVEWESNGQPENNYKTTPPVLDSDGSFFLYSKLTVDKSRWQQGNVFS |
| | CSVMHEALHNHYTQKSLSLSPGKGGGGSGGGGSGGGGSGGGGSGGGGSGGGGSDKTHTCPPCPAPELLGGPSVFLFPPKPKDTLMISR |
| | TPEVTCVVVDVSHEDPEVKFNWYVDGVEVHNAKTKPCEEQYGSTYRCVSVLTVLHQDWLNGKEYKCKVSNKALPAPIEKTISKAKGQP |
| | REPQVYTLPPSREEMTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 251 | QVQLVESGGGVVQPGGSLRLSCAASGFTFSNAWMHWVRQAPGKGLEWVAQIKDKSQNYATYVAESVKGRFTISRADSKNSIYLQMNSL |
| | KTEDTAVYYCRYVHYAAGYGVDIWGQGTTVTVSS |
| 252 | NAWMH |
| 253 | QIKDKSQNYATYVAESVKG |
| 254 | VHYAAGYGVDI |
| 255 | DIVMTQSPLSLPVTPGEPASISCRSSQPLVHSNRNTYLHWYQQKPGQAPRLLIYKVSNRFSGVPDRFSGSGSGTDFTLKISRVEAEDV |
| | GVYYCGQGTQVPYTFGQGTKLEIK |
| 256 | RSSQPLVHSNRNTYLH |
| 257 | KVSNRFS |
| 258 | GQGTQVPYT |
| 259 | VEPKSC |
| 321 | DIVMTQSPLSLPVTPGEPASISCRSSQPLVHSNRNTYLHWYQQKPGQAPRLLIYKVSNRFSGVPDRFSGSGSGTDFTLKISRVEAEDV |
| | GVYYCGQGTQVPYTFGQGTKLEIKSSASTKGPSVFPLAPSSRSTSESTAALGCLVKDYFPEPVTVSWNSGALTSGVHTFPAVLQSSGL |
| | YSLSSVVTVPSSSLGTKTYTCNVDHKPSNTKVDKRVEPKSCGGGGSGGGGSQVQLVESGGGLVQPGRSLRLSCAASGFKFSNVWFHWV |
| | RQAPGKGLEWVAQIKDYYNAYAAYYAPSVKGRFTISRDDSKNSIYLQMNSLKTEDTAVYYCHYVHYASASTLLPAEGVDAWGQGTTVT |
| | VSSASTKGPSVFPLAPSSKSTSGGTAALGCLVEDYFPEPVTVSWNSGALTSGVHTFPAVLQSSGLYSLSSVVTVPSCSLGTQTYICNV |
| | NHKPSNTKVDEKVEPKSCDKTHTCPPCPAPELRGGPKVFLFPPKPKDTLMISRTPEVTCVVVDVSHEDPEVKFNWYVDGVEVHNAKTK |
| | PREEQYASTYRVVSVLTVLHQDWLNGKEYKCKVSNKALPAPIEKTISKAKGQPREPQVYTLPPCREEMTKNQVSLWCLVKGFYPSDIA |
| | VEWESNGQPENNYKTTPPVLDSDGSFFLYSKLTVDKSRWQEGNVFSCSVMHEALHNHYTQKSLSLSP |
| 322 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKKVEPKSCGGGGSGGGGSQVQLVESGGGLVQPGRSLRLSCAASGFKFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSCSLGTQTYICNVNH |
| | KPSNTKVDEKVEPKSCDKTHTCPPCPAPELRRGPKVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCREEMTKNQVSLWCLVKGFYPSDIAVE |
| | WESNGQPENNYKTTPPVLDSDGSFFLYSKLTVDKSRWQEGNVFSCSVMHEALHNHYTQKSLSLSP |
| 323 | DIQMTQSSSSFSVSLGDRVTITCKASEDIYNRLAWYQQKPGNAPRLLISGATSLETGVPSRFSGSGSGKDYTLSITSLQTEDVATYYC |
| | QQYWSTPYTFGGGTKLEVKSSASTKGPSVFPLAPSSKSTSGGTAALGCLVKDYFPEPVTVSWNSGALTSGVHTFPAVLQSSGLYSLSS |
| | VVTVPSSSLGTQTYICNVNHKPSNTKVDKKVEPKSCGGGGSGGGGSQVQLVESGGGLVQPGRSLRLSCAASGFKFSNVWFHWVRQAPG |
| | KGLEWVAQIKDYYNAYAAYYAPSVKGRFTISRDDSKNSIYLQMNSLKTEDTAVYYCHYVHYASASTLLPAEGVDAWGQGTTVTVSSAS |
| | TKGPSVFPLAPSSKSTSGGTAALGCLVEDYFPEPVTVSWNSGALTSGVHTFPAVLQSSGLYSLSSVVTVPSSSLGTQTYICNVNHKPS |
| | NTKVDEKVEPKSCDKTHTCPPCPAPELRRGPKVFLFPPKPKDTLMISRTPEVTCVVVDVSHEDPEVKFNWYVDGVEVHNAKTKPREEQ |
| | YASTYRVVSVLTVLHQDWLNGKEYKCKVSNKALPAPIEKTISKAKGQPREPQVYTLPPCREEMTKNQVSLWCLVKGFYPSDIAVEWES |
| | NGQPENNYKTTPPVLDSDGSFFLYSKLTVDKSRWQEGNVFSCSVMHEALHNHYTQKSLSLSP |
| 324 | DIVMTQSPLSLPVTPGEPASISCRSSQPLVHSNRNTYLHWYQQKPGQAPRLLIYKVSNRFSGVPDRFSGSGSGTDFTLKISRVEAEDV |
| | GVYYCGQGTQVPYTFGQGTKLEIKSSASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCGGGGSGGGGSQVQLVESGGGLVQPGRSLRLSCAASGFKFSNVWFHWV |
| | RQAPGKGLEWVAQIKDYYNAYAAYYAPSVKGRFTISRDDSKNSIVLQMNSLKTEDTAVYYCHYVHYASASTLLPAEGVDAWGQGTTVT |
| | VSSASTKGPSVFPLAPSSKSTSGGTAALGCLVEDYFPEPVTVSWNSGALTSGVHTFPAVLQSSGLYSLSSVVTVPSCSLGTQTYICNV |
| | NHKPSNTKVDEKVEPKSCDKTHTCPPCPAPELRGGPKVFLFPPKPKDTLMISRTPEVTCVVVDVSHEDPEVKFNWYVDGVEVHNAKTK |
| | PREEQYASTYRVVSVLTVLHQDWLNGKEYKCKVSNKALPAPIEKTISKAKGQPREPQVYTLPPCREEMTKNQVSLWCLVKGFYPSDIA |
| | VEWESNGQPENNYKTTPPVLDSDGSFFLYSKLTVDKSRWQEGNVFSCSVMHEALHNHYTQKSLSLSP |
| 325 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSGGGGSQVQLVESGGGLVQPGRSLRLSCAASGFKFSNVWFHWVRQ |
| | APGKGLEWVAQIKDYYNAYAAYYAPSVKGRFTISRDDSKNSIYLQMNSLKTEDTAVYYCHYVHYASASTLLPAEGVDAWGQGTTVTVS |
| | SASTKGPSVFPLAPSSKSTSGGTAALGCLVEDYFPEPVTVSWNSGALTSGVHTFPAVLQSSGLYSLSSVVTVPSSSLGTQTYICNVNH |
| | KPSNTKVDEKVEPKSCDKTHTCPPCPAPEAAGGPSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQQGNVFSCSVLHEALHAHYTRKELSLSP |
| 326 | DIQLTQSPSFLSASVGDRVTITCQSTESVYGSDWLSWYQQKPGQPPKLLIYQASNLEIGVPSRFSGSGSGTDFTLTINSLEAEDAATY |
| | YCQGYYSGYIYAFGGGTKVEIKSSASTKGPSVFPLAPSSRSTSESTAALGCLVKDYFPEPVTVSWNSGALTSGVHTFPAVLQSSGLYS |
| | LSSVVTVPSSSLGTKTYTCNVDHKPSNTKVDKRVEPKSCGGGGSQVQLVESGGGLVQPGRSLRLSCAASGFKFSNVWFHWVRQAPGKG |
| | LEWVAQIKDYYNAYAAYYAPSVKGRFTISRDDSKNSIYLQMNSLKTEDTAVYYCHYVHYASASTLLPAEGVDAWGQGTTVTVSSASTK |
| | GPSVFPLAPSSKSTSGGTAALGCLVEDYFPEPVTVSWNSGALTSGVHTFPAVLQSSGLYSLSSVVTVPSSSLGTQTYICNVNHKPSNT |
| | KVDEKVEPKSCDKTHTCPPCPAPEAAGGPSVFLFPPKPKDTLMISRTPEVTCVVVDVSHEDPEVKFNWYVDGVEVHNAKTKPREEQYA |
| | STYRVVSVLTVLHQDWLNGKEYKCKVSNKALPAPIEKTISKAKGQPREPQVYTLPPCRDELTKNQVSLWCLVKGFYPSDIAVEWESNG |
| | QPENNYKTTPPVLDSDGSFFLYSKLTVDKSRWQQGNVFSCSVLHEALHAHYTRKELSLSP |
| 327 | QVQLVQSGAEVKKPGASVTVSCKASGYTFTDYEMHWIRQPPGEGLEWIGAIDGPTPDTAYSEKFKGRVTLTADKSTSTAYMELSSLTS |
| | EDTAVYYCTRFYSYTYWGQGTLVTVSSASVAAPSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 328 | QVQLVESGGGLVQPGRSLRLSCAASGFKFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSCSLGFQTYICNVNHKPSNTKVDEKVEPKSCDKTHTCPPCPAPELRRGPKVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSREEMTKNQVSLSCAVKGFYPSDIAVEWESNGQPENNYKTTPPVLDSDGSFFLVSKLTVDKSRWQEGNVFSCSVMH |
| | EALHNHYTQKSLSLSP |
| 329 | QVQLVESGGGLVQPGRSLRLSCAASGFKFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSSSLGTQTYICNVNHKPSNTKVDEKVEPKSCDKTHTCPPCPAPELRRGPKVFLFPPKPKDTLMI |
| | SRTPEVTCVVVDVSHEDPEVKFNWYVDGVEVHNAKTKPREEQYASTYRVVSVLTVLHQDWLNGKEYKCKVSNKALPAPIEKTISKAKG |
| | QPREPQVCTLPPSREEMTKNQVSLSCAVKGFYPSDIAVEWESNGQPENNYKTTPPVLDSDGSFFLVSKLTVDKSRWQEGNVFSCSVMH |
| | EALHNHYTQKSLSLSP |
| 330 | QVQLVESGGGLVQPGRSLRLSCAASGFKFSNVWFHWVRQAPGKGLEWVAQIKDYYNAYAAYYAPSVKGRFTISRDDSKNSIYLQMNSL |
| | KTEDTAVYYCHYVHYASASTLLPAEGVDAWGQGTTVTVSSASTKGPSVFPLAPSSKSTSGGTAALGCLVEDYFPEPVTVSWNSGALTS |
| | GVHTFPAVLQSSGLYSLSSVVTVPSSSLGTQTYICNVNHKPSNTKVDEKVEPKSCDKTHTCPPCPAPEAAGGPSVFLFPPKPKDTLMI |
| | SRTPEVTCVVVDVSHEDPEVKDNWYVDGVEVHNAKTKPREEQYASTYRVVSVLTVLHQDWLNGKEYKCKVSNKALPAPIEKTISKAKG |
| | QPREPQVCTLPPSRDELTKNQVSLSCAVKGFYPSDIAVEWESNGQPENNYKTTPPVLDSDGSFFLVSKLTVDKSRWQQGNVFSCSVLH |
| | EALHAHYTRKELSLSP |
| 331 | QVQLVQSGAEVKKPGASVTVSCKASGYTFTDYEMHWIRQPPGLEGLEWIGAIDGPTPDTAYSEKFKGRVTLTADKSTATAYMELSSLT |
| | SEDTAVYYCTRFYSYTYWGQGTLVTVSS |
| 332 | DYEMH |
| 333 | AIDGPTPDTAYSEKFKG |
| 334 | FYSYTY |
| 335 | DIVMTQSPLSLPVTPGEPASISCRSSQPLVHSNRNTYLHWYQQKPGQAPRLLIYKVSNRFSGVPDRFSGSGSGTDFTLKISRVEAEDV |
| | GVYYCGQGTQVPYTFGQGTKLEIK |
| 336 | RSSQPLVHSNRNTYLH |
| 337 | KVSNRFS |
| 338 | GQGTQVPYT |
| 338 | DIVMTQSPLSLPVTPGEPASISVRSSQPLVHSNRNTYLHWYQQKPGQAPRLLIYKVSNRFSGVPDRFSGSGSGTDFTLKISRVEAEDV |
| | GVYYCGQGTQVPYTFGQGTKLEIKSSASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCGGGGSGGGGSQVQLVESGGGLVQPGRSLRLSCAASGFKFSNVWFHWV |
| | RQAPGKGLEWVAQIKDYYNAYAAYYAPSVKGRFTISRDDSKNSIYLQMNSLKTEDTAVYYCHYVHYASASTLLPAEGVDAWGQGTTVT |
| | VSSASTKGPSVFPLAPSSKSTSGGTAALGCLVEDYFPEPVTVSWNSGALTSGVHTFPAVLQSSLGYSLSSVVTVPSSSLGTQTYICNV |
| | NHKPSNTKVDEKVEPKSCDKTHTCPPCPAPELRRGPKVFLFPPKPKDTLMISRTPEVTCVVVDVSHEDPEVKFNWYVDGVEVHANKTK |
| | PREEQYASTYRVVSVLTVLHQDWLNGKEYKCKVSNKALPAPIEKTISKAKGQPREPQVYTLPPCREEMTKNQVSLWCLVKGFYPSDIA |
| | VEWESNGQPENNYKTTPPVLDSDGSFFLYSKLTVDKSRWQEGNVFSCSVMHELAHNHYTQKSLSLSP |

### [Example 3] Affinity Chromatography Approach to Separate Dual/LINC without the “Paired Cysteines” Formation from the Antibody Preparation

### Example 3.1 Concept of Conformation-Specific Antibody that Specifically Binds to Antibody with “Unpaired Cysteines” Form (without Engineered Disulfide Bond) Only

As described in Example 2, an antibody preparation with engineered cysteine (e.g. trivalent 1+2 antibodies shown in FIG. 1, Table 2) comprises heterogenous population of antibody isoforms with engineered disulfide bond (“paired cysteine” or “LINC” form) and without the engineered disulfide bond (“unpaired cysteines” or “unLINC” form). To separate or remove these antibodies without the engineered disulfide bond (“unpaired cysteines” or “unLINC” form) from the antibody preparation, conformation-specific antibody that can specifically bind and/or capture the antibodies of “unpaired cysteines” form but does not bind the antibodies of “paired cysteines” form, can be generated as a tool antibody for use in affinity chromatography purification, analytical and/or quantification applications.

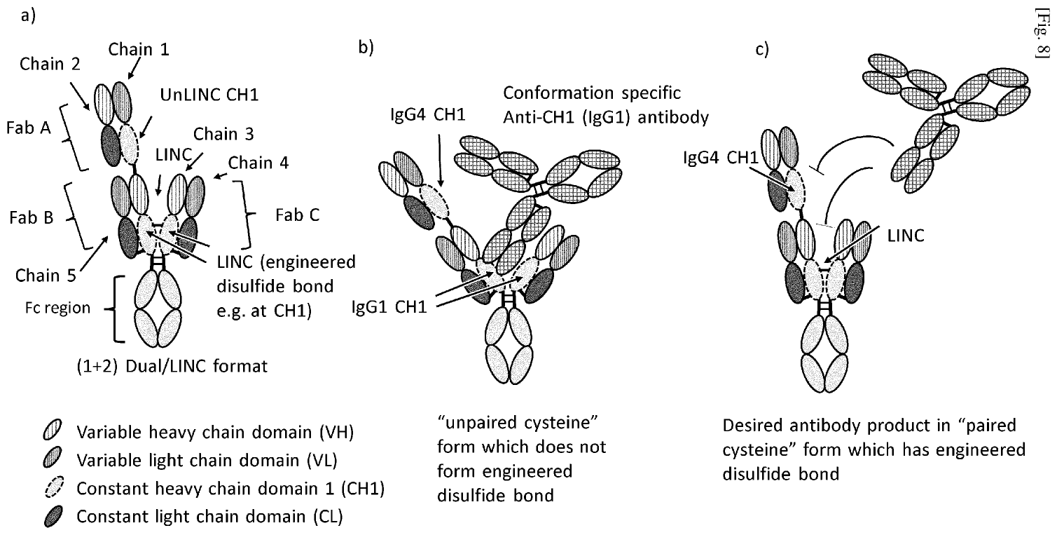

In one embodiment, the target antibody is in IgG (1+1) format comprising an engineered cysteine at each of the two Fabs, and such conformation-specific antibody is an antibody which specifically binds/recognizes to epitope(s) that is/are only accessible to the conformation-specific antibody when the target antibody does not have engineered disulfide bond (“unpaired cysteines” form), wherein such epitope(s) is/are not accessible to the conformation-specific antibody when the target antibody has engineered disulfide bond (“paired cysteine” form) due to e.g. steric hindrance or reduced distance between the two Fabs (FIG. 7). In one preferred embodiment, the engineered cysteine is located at CH1 region of each of the two Fabs, and such epitope(s) is/are located within CH1 region that is/are only accessible to the conformation-specific antibody when the target antibody has “unpaired cysteines” form, wherein such epitope(s) is/are not accessible to the conformation-specific antibody when the target antibody has “paired cysteine” form due to the steric hindrance or reduced distance between the two Fabs (FIG. 7).

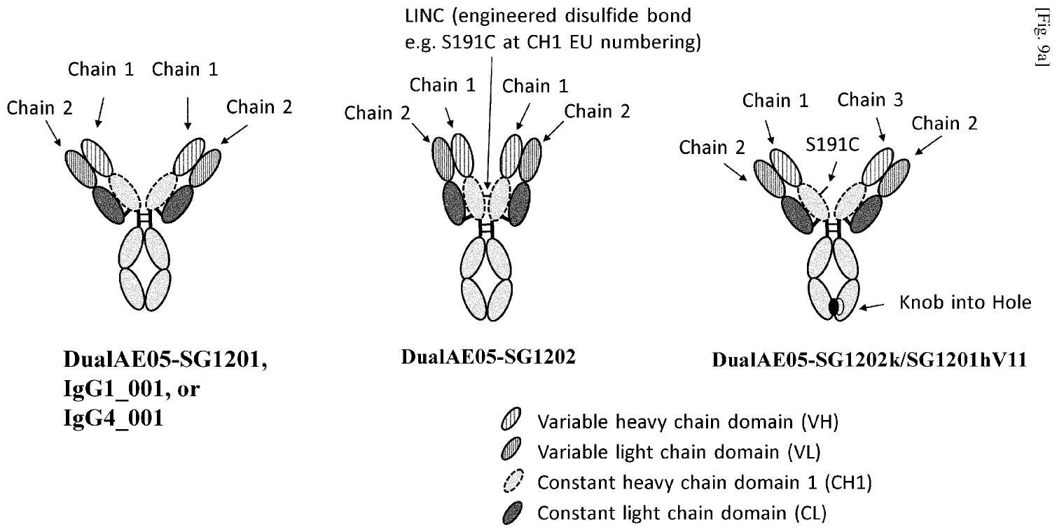

In yet another embodiment, the target antibody is a trivalent 1+2 antibody referred to as Dual/LINC, 1+2 shown in FIG. 8 which comprises three Fab moieties—wherein two of the Fabs (i.e. Fab B and C, comprised in Chain 1-Chain 5 and Chain 3-Chain 4, respectively) each comprises an engineered cysteine which is capable of forming engineered disulfide bond linking both Fabs, and hence can exist in either “unpaired cysteines” or “unLINC” form, or “paired cysteines” or “LINC” form; and one Fab (Fab A, comprised in Chain 1-Chain 2) which does not comprise engineered cysteine which can only exist in “unpaired cysteines” or “unLINC” form. In order to separate antibody with the Dual/LINC, 1+2 format having “unpaired cysteines” or “unLINC” form from the antibody preparation, the conformation-specific antibodies were further selected to specifically bind to an epitope which is unique to the two Fabs (Fab B and C, comprised in Chain 1-Chain 5 and Chain 3-Chain 4, respectively) which can exist in either “unpaired cysteines” form or “paired cysteines” form and is not present in the other Fab (Fab A, comprised in Chain 1-Chain 2) which does not comprise engineered cysteine (only exists in “paired cysteines” form). In one preferred embodiment, the two Fabs (Fab B and C, comprised in Chain 1-Chain 5 and Chain 3-Chain 4, respectively) which can exist in either “unpaired cysteines” form or “paired cysteines” form each comprises a CH1 domain of a first human IgG subclass (e.g. human IgG1 CH1) whereas the other Fab (Fab A, comprised in Chain 1-Chain 2) comprises a CH1 domain of other IgG subclasses different from the first human IgG subclass (e.g. human IgG4 CH1). In such preferred embodiment, the conformation-specific antibody was generated and selected to only specifically bind to CH1 domain of a first human IgG subclass (e.g. human IgG1 CH1) which is in “unpaired cysteines” form (FIG. 8b), but does not bind to CH1 domain of a first human IgG subclass (e.g. human IgG1 CH1) which is in “paired cysteines” form or a CH1 domain of other IgG subclasses different from the first human IgG subclass (e.g. human IgG4 CH1) (FIG. 8c).

### Example 3.2 Generation of Conformation-Specific Anti-CH1 Antibodies that Specifically Bind to CH1 of Dual/LINC 1+2 Antibodies in “Unpaired Cysteines” Form

Anti-CH1 antibodies were prepared, selected and expressed as follows:

Six NZW rabbits were immunized intradermally with an engineered human IgG1 Fab. Four repeated doses were given over a 2-month period followed by blood and spleen collection. B-cells that can bind to engineered human IgG were sorted using a cell sorter and then plated and cultured according to the procedure described in WO2016098356A1. After cultivation, the B cell culture supernatants were collected for further analysis and the B cell pellets were cryopreserved. Binding to recombinant IgG1 with kappa light chain and recombinant IgG with lambda light chain was evaluated by ELISA using the B cell culture supernatants. B cells which can bind to both recombinant IgG1 with kappa light chain and recombinant IgG with lambda light chain were preferred and selected for gene cloning.

RNAs of selected B cell lines with desired binding characters were purified from its cryopreserved cell pellet using ZR-96 Quick-RNA kits (ZYMO RESEARCH, Cat No. R1053). DNAs encoding antibody heavy chain variable regions in the selected lines were amplified by reverse transcription PCR and recombined with DNA encoding rabbit IgG heavy chain constant region. DNAs encoding antibody light chain variable regions were also amplified by reverse transcription PCR and recombined with DNA encoding rabbit kappa light chain constant region. Cloned antibodies were expressed and purified from culture supernatants following the procedure described above.

### Example 3.3 Identification of Conformation-Specific Anti-CH1 Antibodies that Specifically Bind to CH1 of Dual/LINC 1+2 Antibodies in “Unpaired Cysteines” Form

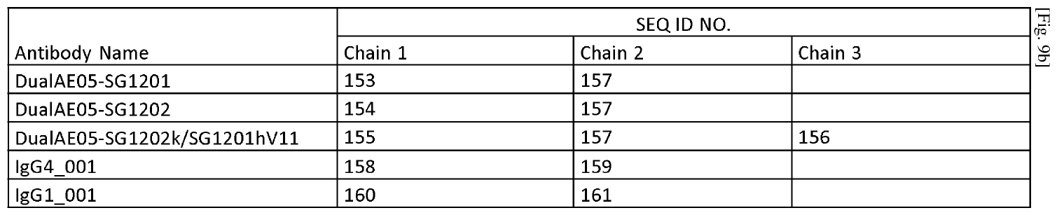

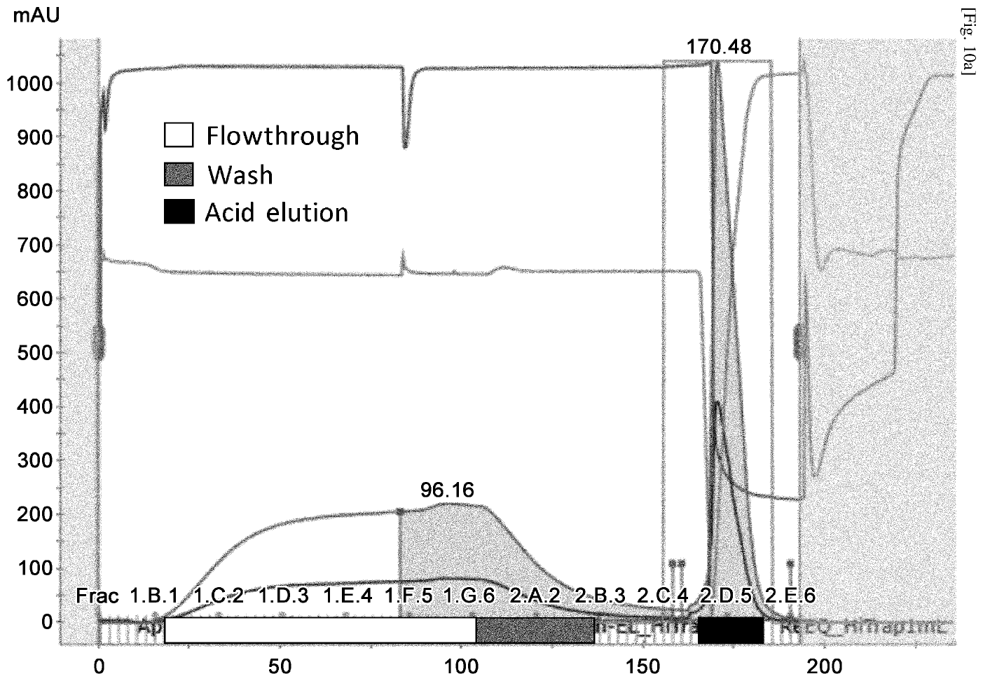

In order to screen and identify conformation-specific anti-CH1 antibodies, the following five antibodies were generated as screening tools. Antibody formats of the five antibodies are illustrated in (FIG. 9a) and the amino acid sequences of polypeptide chains of the antibodies are shown in (FIG. 9b):
(1) and (2) Each of IgG1_001 and DualAE05-SG1201 is a bivalent antibody with human IgG1 CH1 without S191C cysteine substitution (FIG. 9a left panel). This antibody was used to screen and identify anti-CH1 antibodies which specifically bind to CH1 domain of human IgG1 (without engineered disulfide bond at position 191 EU numbering) but do not bind to CH1 domain of human IgG4 (without engineered disulfide bond at position 191 EU numbering).
(3) DualAE05-SG1202 corresponds to DualAE05-SG1201 with S191C cysteine substitution (FIG. 9a middle panel). It corresponds to an antibody which is capable of forming engineered disulfide bond linking both Fabs via S191C, and hence can exist in either “unpaired cysteines” form or “paired cysteines” form.
(4) DualAE05-SG1202k/SG1201hV11 corresponds to a bispecific antibody with human IgG1 CH1, wherein one of the Fab arms comprises S191C mutation, and the other Fab arm does not comprise S191C mutation (FIG. 9a right panel). The heterodimerization of DualAE05-SG1202k/SG1201hV11 heavy chains was controlled by knob into Hole engineering. It represents an antibody that comprises S191C mutation but is not capable of forming engineered disulfide bond linking both Fabs via S191C, and hence can exist only in “unpaired cysteines” form.
(5) IgG4_001 is a bivalent antibody with human IgG4 CH1 without S191C cysteine substitution (FIG. 9a left panel). This antibody was used to screen and identify anti-CH1 antibodies which specifically bind to CH1 domain of human IgG1 (without engineered disulfide bind) but do not bind to CH1 domain of human IgG4 (without engineered disulfide bind).

DualAE05-SG1201, DualAE05-SG1202, DualAE05-SG1202k/SG1201hV11, IgG1_001 and IgG4_001 antibodies were expressed in Expi293 (Invitrogen) and purified by Protein A purification followed by gel filtration.

Biacore binding experiments were performed to characterize binding activities of the anti-CH1 antibodies prepared in Example 3.2 to DualAE05-SG1201, DualAE05-SG1202, DualAE05-SG1202k/SG1201hV11, IgG1_001 and IgG4_001 at 37 degrees C. using Biacore T200 instrument (GE Healthcare). Specifically, mouse anti-human Fc (GE Healthcare) was immobilized onto all flow cells of a CM4 sensor chip using amine coupling kit (GE Healthcare). Tool antibodies DualAE05-SG1201, DualAE05-SG1202, DualAE05-SG1202k/SG1201hV11, IgG1_001 and IgG4_001 were captured onto flow cell, and then anti-CH1 antibodies obtained from Example 3.2 was injected over all the flow cells. All antibodies were prepared in ACES pH 7.4 containing 20 mM ACES, 150 mM NaCl, 0.05% Tween 20, 0.005% NaN₃. Sensor surface was regenerated each cycle with 3M MgCl₂.

It was determined that anti-CH1 antibodies that substantially bind to IgG1_001, DualAE05-SG1201 and DualAE05-SG1202k/SG1201hV11 but do not substantially bind to each of IgG4_001 and DualAE05-SG1202 would fulfil the screening criteria to obtain conformation-specific antibodies that specifically bind to CH1 domain of human IgG1 in the “unpaired cysteines” form, but do not bind to CH1 domain of human IgG1 which is in “paired cysteines” form or CH1 domain of human IgG4 CH1. As a result of the screening, four antibodies namely FAB0059ff, FAB0060hh, FAB0133hh, and FAB0135hh were found and selected as conformation-specific anti-CH1 antibodies which specifically bind Dual/LINC 1+2 antibodies in “unpaired cysteines” form (Table 5). As shown in Table 4, these conformation-specific anti-CH1 antibodies exhibit relatively strong binding activities to each of IgG1_001, DualAE05-SG1201 and DualAE05-SG1202k/SG1201hV11 (Relative binding activity>0.8) while exhibiting no or relatively weak binding activities to IgG4_001 (Relative binding activity<0.1) and DualAE05-SG1202 (Relative binding activity<0.5).

Table 4 shows the relative binding activity of anti-CH1 antibodies to each of tool antibodies DualAE05-SG1201, DualAE05-SG1202, DualAE05-SG1202k/SG1201hV11, IgG1_001 and IgG4_001. The values of relative binding activity to tool antibody (normalized against RU binding value for IgG1_001) are obtained by dividing the Biacore binding response (RU) for anti-CH1 antibody to tool antibody, to RU binding value for anti-CH1 antibody to IgG1_001.

TABLE 4

| | Relative binding activity to tool antibody | | | | |
|---|---|---|---|---|---|
| Name of | (normalized against RU binding value for IgG1_001) | | | | |
| anti-CH1 | | | DualAE05- | DualAE05- | DualAE05- |
| antibodies | IgG1_001 | IgG4_001 | SG1201 | SG1202 | SG1202k/SG1201hV11 |
| FAB0059ff | 1.000 | 0.007 | 0.952 | 0.493 | 0.929 |
| FAB0060hh | 1.000 | −0.009 | 0.968 | 0.489 | 0.957 |
| FAB0133hh | 1.000 | −0.017 | 1.016 | 0.206 | 0.922 |
| FAB0135hh | 1.000 | −0.023 | 1.007 | 0.243 | 0.960 |

In order to improve the physicochemical properties of the conformation-specific anti-CH1 antibodies, cystine residues at CDR regions were removed from FAB0059ff, FAB0060hh, and FAB0133hh, and subsequently named as FAB0059Hf/FAB0059L0001, FAB0060Hh/FAB0060L0001, and FAB0133Hh/FAB0133L0001, respectively. Amino acid sequence SEQ ID NOs of the conformation-specific anti-CH1 antibodies were shown in Table 5.

TABLE 5

| Name of anti-CH1 | SEQ ID NOs. | | | | | | | |
|---|---|---|---|---|---|---|---|---|
| antibodies | VH | HCDR1 | HCDR2 | HCDR3 | VL | LCDR1 | LCDR2 | LCDR3 |
| FAB0059ff | 162 | 166 | 170 | 174 | 178 | 182 | 186 | 190 |
| FAB0060hh | 163 | 167 | 171 | 175 | 179 | 183 | 187 | 191 |
| FAB0133hh | 164 | 168 | 172 | 176 | 180 | 184 | 188 | 192 |
| FAB0135hh | 165 | 169 | 173 | 177 | 181 | 185 | 189 | 193 |
| FAB0059Hf/FAB0059L0001 | 162 | 166 | 170 | 174 | 196 | 115 | 124 | 134 |
| FAB0060Hh/FAB0060L0001 | 163 | 167 | 171 | 175 | 197 | 116 | 125 | 135 |
| FAB0133Hh/FAB0133L0001 | 164 | 168 | 172 | 176 | 198 | 118 | 128 | 137 |

### Example 3.4 Use of Conformation-Specific Anti-CH1 Antibodies for Removing Dual/LINC 1+2 Antibodies Having “Unpaired Cysteines” Form from the Antibody Preparation

The conformation-specific anti-CH1 antibodies described in Example 3.3 can be used as a ligand or binder to selectively capture or remove Dual/LINC 1+2 antibodies which are in “unpaired cysteines” form from an antibody preparation e.g. harvested from cell culture supernatant. For example, conformation-specific anti-CH1 antibodies can be immobilized to a column for removing Dual/LINC 1+2 antibodies which are in “unpaired cysteines” form from the antibody preparations using affinity purification.

Conformation-specific anti-CH1 antibody, FAB0133Hh/FAB0133L0001; and Dual/LINC 1+2 antibody, DLL3-DualAE05/DualAE05-FF056 were transiently transfected and expressed using Expi293 Expression system (Thermo Fisher Scientific). The format of the DLL3-DualAE05/DualAE05-FF056 has a molecular format shown in FIG. 8a and comprises five polypeptide chains represented by amino acid sequences of SEQ ID NO: 142 (Chain 1), SEQ ID NO: 147 (Chain 2), SEQ ID NO: 148 (Chain 3) and SEQ ID NO: 157 (Chain 4 & 5). Cell culture supernatants were harvested, and antibodies were purified from the supernatants using MabSelect SuRe affinity chromatography (GE Healthcare) followed by gel filtration chromatography using Superdex200 (GE Healthcare).

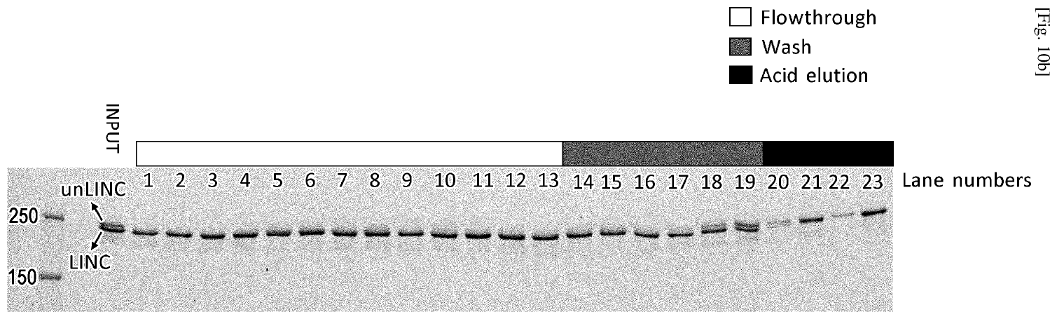

For affinity purification, NHS Sepharose resins conjugated with the purified FAB0133Hh/FAB0133L0001 were packed into XK 16/20 column (GE Healthcare). After protein A chromatography treatment, antibody preparation of DLL3-DualAE05/DualAE05-FF056, was applied to the XK 16/20 column to allow specific capturing/binding of DLL3-DualAE05/DualAE05-FF056 which is in “unpaired cysteines” form onto the column, wherein DLL3-DualAE05/DualAE05-FF056 antibodies which are in “paired cysteines” form will not be captured or bound by the column and appear predominantly in the flow through fractions. Subsequently, the affinity captured DLL3-DualAE05/DualAE05-FF056 which is in “unpaired cysteines” form was eluted by treatment with 50 mM HCl. FIG. 10 shows chromatography profile (FIG. 10a) and non-reducing SDS-PAGE analysis (FIG. 10b) of the eluted antibodies in affinity purification of DLL3-DualAE05/DualAE05-FF056 using conformation-specific anti-CH1 antibody FAB0133Hh/FAB0133L0001 column. Specifically, the flow-through fractions comprise high purity of DualAE05/DualAE05-FF056 which is in “paired cysteines” or “LINC” form (flowthrough: white bar) as indicated by one predominant protein band which migrates faster in the non-reducing SDS-PAGE analysis (Lanes 1 to 13); wash fractions comprise mixture of DualAE05/DualAE05-FF056 which is in “unpaired cysteines” form and DualAE05/DualAE05-FF056 which is in “paired cysteines” form (wash: gray bar, Lanes 14 to 19); and eluted fractions comprise predominantly DualAE05/DualAE05-FF056 which is in “unpaired cysteines” form (acid elution: black bar) as indicated by one predominant protein band which migrates slower in the non-reducing SDS-PAGE analysis (Lanes 20 to 23).

### Example 4 Use of Conformation-Specific Anti-CH1 Antibodies for Quantitative Analysis of Dual/LINC 1+2 Antibodies Having “Unpaired Cysteines” Form

Conformation-specific anti-CH1 antibodies identified in Example 3 such as FAB0133Hh/FAB0133L0001 were used as a tool to perform quantitative analysis to measure the purity or ratio of antibodies which are in “unpaired cysteines” form using analytical methods known in the art such as SPR measurement.

Specifically, DLL3-DualAE05/DualAE05-FF110 was prepared from cell harvest, first treated with Pro A column, and then followed by affinity purification with the anti-CH1 antibody column described in Example 3.4.

DLL3-DualAE05/DualAE05-FF110 sample eluted from Pro A column and DLL3-DualAE05/DualAE05-FF110 sample flowthrough from the anti-CH1 antibody column were collected for Biacore binding analysis using Biacore 8K instrument. A linear correlation relationship with R² of 0.9987 was observed between binding response of FAB0133Hh/FAB0133L0001 to an antibody sample containing “unpaired cysteine” form at various concentration ratio at 2.5%, 5%, 7.5%, 10%, 15%, 20%, 40%, 60%, 80%, 100% by using SPR binding analysis (data not shown). Therefore, percentage (%) amount or ratio of DLL3-DualAE05/DualAE05-FF110 which is in “unpaired cysteines” form in the antibody sample can be calculated by measuring % binding of FAB0133Hh/FAB0133L0001 to the antibody sample. The antibody sample was captured on a CM5 sensor chip coated with anti-human Fc of a llama antibody fragment. 1 micromolar concentration of FAB0133Hh/FAB0133L0001 was injected and binding response of the interaction was measured. Assay temperature was set at 25 degrees C. All antibodies and analytes were prepared in ACES pH 7.4 containing 20 mM ACES, 150 mM NaCl, 0.05% Tween 20, 0.005% NaN₃.

As shown in Table 6, FAB0133Hh/FAB0133L0001 shows reduced binding response to DLL3-DualAE05/DualAE05-FF110 flowthrough sample from the anti-CH1 antibody column, indicating reduced amount of “unpaired cysteines” form (<2%) after purification process with the anti-CH1 antibody column.

TABLE 6

| | % “unpaired |
|---|---|
| Antibody sample | cysteines” form |
| DLL3-DualAE05/DualAE05-FF110 eluted from | 22.61 |
| proA column | |
| DLL3-DualAE05/DualAE05-FF110 flowthrough | 1.07 |
| from the anti-CH1 antibody column | |

### Reference Example 1. Preparation of Antibody Expression Vector and Expression and Purification of Antibody

Amino acid substitution or IgG conversion was carried out by a method generally known to those skilled in the art using PCR, or In-fusion Advantage PCR cloning kit (Takara Bio Inc.), etc., to construct expression vectors. The obtained expression vectors were sequenced by a method generally known to those skilled in the art. The prepared plasmids were transiently transferred to FreeStyle 293 cells (ThermoFisher Scientific) or Expi293F cells (ThermoFisher Scientific) to express antibodies. Each antibody was purified from the obtained culture supernatant by a method generally known to those skilled in the art using rProtein A Sepharose™ Fast Flow (GE Healthcare Japan Corp.). As for the concentration of the purified antibody, the absorbance was measured at 280 nm using a spectrophotometer, and the antibody concentration was calculated by use of an extinction coefficient calculated from the obtained value by PACE (Protein Science 1995; 4: 2411-2423).

### Reference Example 2. Non-Reducing SDS-PAGE to Characterize Purities of Antibodies

Non-reducing SDS-PAGE was performed using 4-20% Mini-PROTEAN (registered trademark) TGX Stain-Free™ Precast Gels (Bio-Rad) with 1×Tris/Glycine/SDS running buffer (Bio-Rad). Monoclonal antibody samples were heated at 70 degrees C. for 10 min. 0.2 microgram was loaded and electrophoresis was conducted at 200 V for 90 min. Proteins were visualized with Chemidoc Imaging System (Bio-Rad). Percentage of individual band is analyzed by the Image Lab software version 6.0 (Bio-Rad), in which % intensity of the individual band e.g. faster migration (Lower band) and slower migration (Upper band) were calculated by intensity of the band divided by the sum of these two bands.

### Reference Example 3

Screening of Affinity Matured Variants of Parental Dual-Fab H183L072 for Improvement in In Vitro Cytotoxicity on Tumor Cells

1.1 Sequence of Affinity Matured Variants

Concept of providing an immunoglobulin variable (Fab) region that binds CD3 and CD137, but does not bind to CD3 and CD137 at same time (Dual-Fab) is disclosed in WO2019111871 (incorporated herein by reference). To increase the binding affinity of parental Dual-Fab H183L072 (Heavy chain: SEQ ID NO: 1; Light chain: SEQ ID NO: 57) disclosed in WO2019111871, more than 1,000 Dual-Fab variants were generated using H183L072 as a template by introduce single or multiple mutations on variable region. Antibodies were expressed Expi293 (Invitrogen) and purified by Protein A purification followed by gel filtration, when gel filtration was necessary. The sequences of 22 represented Dual-Fab variants with multiple mutations are listed in Table 7 and Tables 8-1 to 8-6 and binding affinity and kinetics towards CD3 and CD137 were evaluated in 1.2.2 of Reference Example 3 (Tables 11-1 and 11-2) at 25 degrees C. and/or 37 degrees C. using Biacore T200 instrument (GE Healthcare) described below.

TABLE 7

| DualAE No. | Ab name | VHR name | VLR name | VHR | VHR_CDR1 | VHR_CDR2 |
|---|---|---|---|---|---|---|
| Parent | H183/L072 | dBBDu183H | dBBDu072L | 1 | 15 | 29 |
| DualAE01 | H0868L0581 | dBBDu183H0868 | dBBDu072L0581 | 2 | 16 | 30 |
| DualAE08 | H1550L0918 | dBBDu183H1550 | dBBDu072L0918 | 3 | 17 | 31 |
| DualAE06 | H1571L0581 | dBBDu183H1571 | dBBDu072L0581 | 4 | 18 | 32 |
| DualAE17 | H1610L0581 | dBBDu183H1610 | dBBDu072L0581 | 5 | 19 | 33 |
| DualAE10 | H1610L0939 | dBBDu183H1610 | dBBDu072L0939 | 5 | 19 | 33 |
| DualAE05 | H1643L0581 | dBBDu183H1643 | dBBDu072L0581 | 6 | 20 | 34 |
| DualAE19 | H1647L0581 | dBBDu183H1647 | dBBDu072L0581 | 8 | 22 | 36 |
| DualAE20 | H1649L0581 | dBBDu183H1649 | dBBDu072L0581 | 9 | 23 | 37 |
| DualAE21 | H1649L0943 | dBBDu183H1649 | dBBDu072L0943 | 9 | 23 | 37 |
| DualAE22 | H1651L0581 | dBBDu183H1651 | dBBDu072L0581 | 10 | 24 | 38 |
| DualAE23 | H1652L0943 | dBBDu183H1652 | dBBDu072L0943 | 11 | 25 | 39 |
| DualAE09 | H1673L0943 | dBBDu183H1673 | dBBDu072L0943 | 12 | 26 | 40 |
| DualAE18 | H1673L0581 | dBBDu183H1673 | dBBDu072L0581 | 12 | 26 | 40 |
| DualAE14 | H2591L0581 | dBBDu183H2591 | dBBDu072L0581 | 13 | 27 | 41 |
| DualAE15 | H2594L0581 | dBBDu183H2594 | dBBDu072L0581 | 14 | 28 | 42 |
| DualAE16 | H1644L0939 | dBBDu183H1644 | dBBDu072L0939 | 81 | 82 | 83 |
| DualAE02 | H0888L0581 | dBBDu183H0888 | dBBDu072L0581 | 101 | 114 | 127 |
| DualAE24 | H1595L0581 | dBBDu183H1595 | dBBDu072L0581 | 104 | 117 | 130 |
| DualAE07 | H1573L0581 | dBBDu183H1573 | dBBDu072L0581 | 106 | 119 | 132 |
| DualAE25 | H1579L0581 | dBBDu183H1579 | dBBDu072L0581 | 107 | 120 | 133 |
| DualAE26 | H1572L0581 | dBBDu183H1572 | dBBDu072L0581 | 110 | 123 | 136 |
| DualAE27 | H0883 | dBBDu183H0883 | dBBDu072L | 113 | 126 | 139 |
| | CD3ε | CD3εVH | CD3εVL | 77 | | |
| | CD137 | CD137VH | CD137VL | 79 | | |

| DualAE No. | VHR_CDR3 | VLR | VLR_CDR1 | VLR_CDR2 | VLR_CDR3 |
|---|---|---|---|---|---|
| Parent | 43 | 57 | 62 | 67 | 72 |
| DualAE01 | 44 | 58 | 63 | 68 | 73 |
| DualAE08 | 45 | 59 | 64 | 69 | 74 |
| DualAE06 | 46 | 58 | 63 | 68 | 73 |
| DualAE17 | 47 | 58 | 63 | 68 | 73 |
| DualAE10 | 47 | 60 | 65 | 70 | 75 |
| DualAE05 | 48 | 58 | 63 | 68 | 73 |
| DualAE19 | 50 | 58 | 63 | 68 | 73 |
| DualAE20 | 51 | 58 | 63 | 68 | 73 |
| DualAE21 | 51 | 61 | 66 | 71 | 76 |
| DualAE22 | 52 | 58 | 63 | 68 | 73 |
| DualAE23 | 53 | 61 | 66 | 71 | 76 |
| DualAE09 | 54 | 61 | 66 | 71 | 76 |
| DualAE18 | 54 | 58 | 63 | 68 | 73 |
| DualAE14 | 55 | 58 | 63 | 68 | 73 |
| DualAE15 | 56 | 58 | 63 | 68 | 73 |
| DualAE16 | 84 | 60 | 65 | 70 | 75 |
| DualAE02 | 140 | 58 | 63 | 68 | 73 |
| DualAE24 | 143 | 58 | 63 | 68 | 73 |
| DualAE07 | 145 | 58 | 63 | 68 | 73 |
| DualAE25 | 146 | 58 | 63 | 68 | 73 |
| DualAE26 | 149 | 58 | 63 | 68 | 73 |
| DualAE27 | 152 | 57 | 62 | 67 | 72 |
| | | 78 | | | |
| | | 80 | | | |

TABLE 8-1

| | SEQ | |
|---|---|---|
| SEQ list | number | Amino Acid Sequence |
| dBBDu183H | 1 | QVQLVESGGGLVQPGRSLRLSCAASGFTFSNAWMHWVRQAPGKGLEWVAQIKDKGNAYAAYYAP |
| | | SVKGRFTISRDDSKNSIYLQMNSLKTEDTAVYYCHYVHYASASTVLPAFGVDAWGQGTTVTVSS |
| dBBDu183H0868 | 2 | QVQLVESGGGLVQPGRSLRLSCAASGFKFSNVWMHWVRQAPGKGLEWVAQIKDKYNAYAAYYAP |
| | | SVKGRFTISRDDSKNSIYLQMNSLKTEDTAVYYCHYVHYASASTLLPAFGVDAWGQGTTVTVSS |
| dBBDu183H1550 | 3 | QVQLVESGGGLVQPGRSLRLSCAASGFKFSNVWMHWVRQAPGKGLEWVAQIKDKYNAYAAYYAP |
| | | SVKGRFTISRDDSKNSIYLQMNSLKTEDTAVYYCHYIHYASASTLLPAFGVDAWGQGTTVTVSS |
| dBBDu183H1571 | 4 | QVQLVESGGGLVQPGRSLRLSCAASGFKFSNVWFHWVRQAPGKGLEWVAQIKDKYNAYATYYAP |
| | | SVKGRFTISRDDSKNSIYLQMNSLKTEDTAVYYCHYVHYASASTLLPAFGVDAWGQGTTVTVSS |
| dBBDu183H1610 | 5 | QVQLVESGGGLVQPGRSLRLSCAASGFVFSNVWMHWVRQAPGKGLEWVAQIKDKWNAYAAYYAP |
| | | SVKGRFTISRDDSKNSIYLQMNSLKTEDTAVYYCHYIHYASASTLLPAEGIDAWGQGTTVTVSS |
| dBBDu183H1643 | 6 | QVQLVESGGGLVQPGRSLRLSCAASGFKFSNVWFHWVRQAPGKGLEWVAQIKDYYNAYAAYYAP |
| | | SVKGRFTISRDDSKNSIYLQMNSLKTEDTAVYYCHYVHYASASTLLPAEGVDAWGQGTTVTVSS |
| dBBDu183H1647 | 8 | QVQLVESGGGLVQPGRSLRLSCAASGFKFSNTWFHWVRQAPGKGLEWVAQIKDYYNDYAAYYAP |
| | | SVKGRFTISRDDSKNSIYLQMNSLKTEDTAVYYCHYVHYASASTLLPAEGVDAWGQGTTVTVSS |
| dBBDu183H1649 | 9 | QVQLVESGGGLVQPGRSLRLSCAASGFVFSNVWFHWVRQAPGKGLEWVAQIKDKYANYADYYAP |
| | | SVKERFTISRDDSKNSIYLQMNSLKTEDTAVYYCHYVHYASASTLLPAEGVDAWGQGTTVTVSS |
| dBBDu183H1651 | 10 | QVQLVESGGGLVQPGRSLRLSCAASGFVFSNVWFHWVRQAPGKGLEWVAQIKDKYNAYADYYAP |
| | | SVEGRFTISRDDSKNSIYLQMNSLKTEDTAVYYCHYVHYASASTLLPAEGVDAWGQGTTVTVSS |
| dBBDu183H1652 | 11 | QVQLVESGGGLVQPGRSLRLSCAASGFVFSNVWFHWVRQAPGKGLEWVAQIKDYYNAYADYYAP |
| | | SVEGRFTISRDDSKNSIYLQMNSLKTEDTAVYYCHYVHYASASTLLPAEGVDAWGQGTTVTVSS |
| dBBDu183H1673 | 12 | QVQLVESGGGLVQPGRSLRLSCAASGFVFSNVWFHWVRQAPGKGLEWVAQIKDKWNAYADYYAP |
| | | SVKERFTISRDDSKNSIYLQMNSLKTEDTAVYYCHYIHYASASTLLPAEGIDAWGQGTTVTVSS |

TABLE 8-2

| | SEQ | |
|---|---|---|
| SEQ list | number | Amino Acid Sequence |
| dBBDu183H2591 | 13 | QVQLVESGGGLVQPGRSLRLSCAASGFKFSNVWFHWVRQAPGKGLEWVAQIKDYYNAYAGYYHP |
| | | SVKGRFTISRDDSKNSIYLQMNSLKTEDTAVYYCHYVHYAAASTLLPAEGVDAWGQGTTVTVSS |
| dBBDu183H2594 | 14 | QVQLVESGGGLVQPGRSLRLSCAASGFKFSNVWFHWVRQAPGKGLEWVAQIKDYYNAYAGYYHP |
| | | SVKGRFTISRDDSKNSIYLQMNSLKTEDTAVYYCHYVHYAAASQLLPAEGVDAWGQGTTVTVSS |
| dBBDu183H_VHR_CDR1 | 15 | NAWMH |
| dBBDu183H0868_VHR_CDR1 | 16 | NVWMH |
| dBBDu183H1550_VHR_CDR1 | 17 | NVWMH |
| dBBDu183H1571_VHR_CDR1 | 18 | NVWFH |
| dBBDu183H1610_VHR_CDR1 | 19 | NVWMH |
| dBBDu183H1643_VHR_CDR1 | 20 | NVWFH |
| dBBDu183H1647_VHR_CDR1 | 22 | NTWFH |
| dBBDu183H1649_VHR_CDR1 | 23 | NVWFH |
| dBBDu183H1651_VHR_CDR1 | 24 | NVWFH |
| dBBDu183H1652_VHR_CDR1 | 25 | NVWFH |
| dBBDu183H1673_VHR_CDR1 | 26 | NVWFH |
| dBBDu183H2591_VHR_CDR1 | 27 | NVWFH |
| dBBDu183H2594_VHR_CDR1 | 28 | NVWFH |
| dBBDu183H_VHR_CDR2 | 29 | QIKDKGNAYAAYYAPSVKG |
| dBBDu183H0868_VHR_CDR2 | 30 | QIKDKYNAYAAYYAPSVKG |
| dBBDu183H1550_VHR_CDR2 | 31 | QIKDKYNAYAAYYAPSVKG |
| dBBDu183H1571_VHR_CDR2 | 32 | QIKDKYNAYATYYAPSVKG |

TABLE 8-3

| | SEQ | |
|---|---|---|
| SEQ list | number | Amino Acid Sequence |
| dBBDu183H1610_VHR_CDR2 | 33 | QIKDKWNAYAAYYAPSVKG |
| dBBDu183H1643_VHR_CDR2 | 34 | QIKDYYNAYAAYYAPSVKG |
| dBBDu183H1647_VHR_CDR2 | 36 | QIKDYYNDYAAYYAPSVKG |
| dBBDu183H1649_VHR_CDR2 | 37 | QIKDKYNAYADYYAPSVKE |
| dBBDu183H1651_VHR_CDR2 | 38 | QIKDKYNAYADYYAPSVEG |
| dBBDu183H1652_VHR_CDR2 | 39 | QIKDYYNAYADYYAPSVEG |
| dBBDu183H1673_VHR_CDR2 | 40 | QIKDKWNAYADYYAPSVKE |
| dBBDu183H2591_VHR_CDR2 | 41 | QIKDYYNAYAGYYHPSVKG |
| dBBDu183H2594_VHR_CDR2 | 42 | QIKDYYNAYAGYYHPSVKG |
| dBBDu183H_VHR_CDR3 | 43 | VHYASASTVLPAFGVDA |
| dBBDu183H0868_VHR_CDR3 | 44 | VHYASASTLLPAFGVDA |
| dBBDu183H1550_VHR_CDR3 | 45 | IHYASASTLLPAFGVDA |
| dBBDu183H1571_VHR_CDR3 | 46 | VHYASASTLLPAFGVDA |
| dBBDu183H1610_VHR_CDR3 | 47 | IHYASASTLLPAEGIDA |
| dBBDu183H1643_VHR_CDR3 | 48 | VHYASASTLLPAFGVDA |
| dBBDu183H1647_VHR_CDR3 | 50 | VHYASASTLLPAEGVDA |
| dBBDu183H1649_VHR_CDR3 | 51 | VHYASASTLLPAFGVDA |
| dBBDu183H1651_VHR_CDR3 | 52 | VHYASASTLLPAFGVDA |
| dBBDu183H1652_VHR_CDR3 | 53 | VHYASASTLLPAFGVDA |
| dBBDu183H1673_VHR_CDR3 | 54 | IHYASASTLLPAEGIDA |
| dBBDu183H2591_VHR_CDR3 | 55 | VHYAAASTLLPAEGVDA |
| dBBDu183H2594_VHR_CDR3 | 56 | VHYAAASQLLPAEGVDA |

TABLE 8-4

| | SEQ | |
|---|---|---|
| SEQ list | number | Amino Acid Sequence |
| dBBDu072L | 57 | DIVMTQSPLSLPVTPGEPASISCQASQELVHMNRNTYLHWYQQKPGQAPRLLIYKVSNRFPGVPD |
| | | RFSGSGSGTDFTLKISRVEAEDVGVYYCAQGTSVPFTFGQGTKLEIK |
| dBBDu072L0581 | 58 | DIVMTQSPLSLPVTPGEPASISCQPSQEVVHMNRNTYLHWYQQKPGQAPRLLIYKVSNRFPGVPD |
| | | RFSGSGSGTDFTLKISRVEAEDVGVYYCAQGTSHPFTFGQGTKLEIK |
| dBBDu072L0918 | 59 | DIVMTQSPLSLPVTPGEPASISCQPSQEVVHMNNVVYLHWYQQKPGQAPRLLIYKVSNRFPGVPD |
| | | RFSGSGSGTDFTLKISRVEAEDVGVYYCAQGTSHPFTFGQGTKLEIK |
| dBBDu072L0939 | 60 | DIVMTQSPLSLPVTPGEPASISCQPSQEVVHMNRNTYLHWYQQKPGQAPRLLIYKVSNVFPGVPD |
| | | RFSGSGSGTDFTLKISRVEAEDVGVYYCAQGTHHPFTFGQGTKLEIK |
| dBBDu072L0943 | 61 | DIVMTQSPLSLPVTPGEPASISCQPSEEVVHMNRNTYLHWYQQKPGQAPRLLIYKVSNLFPGVPD |
| | | RFSGSGSGTDFTLKISRVEAEDVGVYYCAQGTHHPFTFGQGTKLEIK |
| dBBDu072L_VLR_CDR1 | 62 | QASQELVHMNRNTYLH |
| dBBDu072L0581_VLR_CDR1 | 63 | QPSQEVVHMNRNTYLH |
| dBBDu072L0918_VLR_CDR1 | 64 | QPSQEVVHMNNVVYLH |
| dBBDu072L0939_VLR_CDR1 | 65 | QPSQEVVHMNRNTYLH |
| dBBDu072L0943_VLR_CDR1 | 66 | QPSEEVVHMNRNTYLH |
| dBBDu072L_VLR_CDR2 | 67 | KVSNRFP |
| dBBDu072L0581_VLR_CDR2 | 68 | KVSNRFP |
| dBBDu072L0918_VLR_CDR2 | 69 | KVSNRFP |
| dBBDu072L0939_VLR_CDR2 | 70 | KVSNVFP |
| dBBDu072L0943_VLR_CDR2 | 71 | KVSNLFP |
| dBBDuO72L_VLR_CDR3 | 72 | AQGTSVPFT |
| dBBDu072L0581_VLR_CDR3 | 73 | AQGTSHPFT |
| dBBDu072L0918_VLR_CDR3 | 74 | AQGTSHPFT |
| dBBDu072L0939_VLR_CDR3 | 75 | AQGTHHPFT |
| dBBDu072L0943_VLR_CDR3 | 76 | AQGTHHPFT |

TABLE 8-5

| | SEQ | |
|---|---|---|
| SEQ list | number | Amino Acid Sequence |
| CD3ϵVH | 77 | QVQLVESGGGVVQPGGSLRLSCAASGFTFSNAWMHWVRQAPGKGLEWVAQIKDKSQNYATYV |
| | | AESVKGRFTISRADSKNSIYLQMNSLKTEDTAVYYCRYVHYAAGYGVDIWGQGTTVTVSS |
| CD3ϵVL | 78 | DIVMTQSPLSLPVTPGEPASISCRSSQPLVHSNRNTYLHWYQQKPGQAPRLLIYKVSNRFSG |
| | | VPDRFSGSGSGTDFTLKISRVEAEDVGVYYCGQGTQVPYTFGQGTKLEIK |
| CD137VH | 79 | QVQLQQWGAGLLKPSETLSLTCAVYGGSFSGYYWSWIRQSPEKGLEWIGEINHGGYVTYNPS |
| | | LESRVTISVDTSKNQFSLKLSSVTAADTAVYYCARDYGPGNYDWYFDLWGRGTLVTVSS |
| CD137VL | 80 | EIVLTQSPATLSLSPGERATLSCRASQSVSSYLAWYQQKPGQAPRLLIYDASNRATGIPARF |
| | | SGSGSGTDFTLTISSLEPEDFAVYYCQQRSNWPPALTFGGGTKVEIK |
| dBBDu183H1644 | 81 | QVQLVESGGGLVQPGRSLRLSCAASGFVFSNVWFHWVRQAPGKGLEWVAQIKDYYNAYAAYY |
| | | APSVKGRFTISRDDSKNSIYLQMNSLKTEDTAVYYCHYVHYASASTLLPAEGVDAWGQGTTV |
| | | TVSS |
| dBBDu183H1644_VHR_CDR1 | 82 | NVWFH |
| dBBDu183H1644_VHR_CDR2 | 83 | QIKDYYNAYAAYYAPSVKG |
| dBBDu183H1644_VHR_CDR3 | 84 | VHYASASTLLPAEGVDA |
| dBBDu183H0888 | 101 | QVQLVESGGGLVQPGRSLRLSCAASGFKFSNVWMHWVRQAPGKGLEWVAQIKDKWNAYAAYY |
| | | APSVKGRFTISRDDSKNSIYLQMNSLKTEDTAVYYCHYIHYASASTLLPAFGIDAWGQGTTV |
| | | TVSS |
| dBBDu183H1595 | 104 | QVQLVESGGGLVQPGRSLRLSCAASGFKFSNTWMHWVRQAPGKGLEWVAQIKDKYNAYAAYY |
| | | APSVKGRFTISRDDSKNSIYLQMNSLKTEDTAVYYCHYIHYASASTLLPAFGVDAWGQGTTV |
| | | TVSS |
| dBBDu183H1573 | 106 | QVQLVESGGGLVQPGRSLRLSCAASGFKFSNVWFHWVRQAPGKGLEWVAQIKDYYNAYAAYY |
| | | APSVKGRFTISRDDSKNSIYLQMNSLKTEDTAVYYCHYVHYASASTLLPAFGVDAWGQGTTV |
| | | TVSS |
| dBBDu183H1579 | 107 | QVQLVESGGGLVQPGRSLRLSCAASGFKFSHVWFHWVRQAPGKGLEWVAQIKDKYNAYAAYY |
| | | APSVKGRFTISRDDSKNSIYLQMNSLKTEDTAVYYCHYVHYASASTLLPAFGVDAWGQGTTV |
| | | TVSS |
| dBBDu183H1572 | 110 | QVQLVESGGGLVQPGRSLRLSCAASGFKFSNVWFHWVRQAPGKGLEWVAQIKDKYNAYAAYY |
| | | APSVKGRFTISRDDSKNSIYLQMNSLKTEDTAVYYCHYVHYASASTLLPAEGVDAWGQGTTV |
| | | TVSS |
| dBBDu183H0883 | 113 | QVQLVESGGGLVQPGRSLRLSCAASGFTFSNAWMHWVRQAPGKGLEWVAQIKDKGNAYAAYY |
| | | APSVKGRFTISRDDSKNSIYLQMNSLKTEDTAVYYCRYVHYASASTLLPAFGVDAWGQGTTV |
| | | TVSS |

TABLE 8-6

| | SEQ | |
|---|---|---|
| SEQ list | number | Amino Acid Sequence |
| dBBDu183H0888_VHR_CDR1 | 114 | NVWMH |
| dBBDu183H1495_VHR_CDR1 | 117 | NTWMH |
| dBBDu183H1573_VHR_CDR1 | 119 | NVWFH |
| dBBDu183H1579_VHR_CDR1 | 120 | HVWFH |
| dBBDu183H1572_VHR_CDR1 | 123 | NVWFH |
| dBBDu183H0883_VHR_CDR1 | 126 | NAWMH |
| dBBDu183H0888_VHR_CDR2 | 127 | QIKDKWNAYAAYYAPSVKG |
| dBBDu183H1595_VHR_CDR2 | 130 | QIKDKYNAYAAYYAPSVKG |
| dBBDu183H1573_VHR_CDR2 | 132 | QIKDYYNAYAAYYAPSVKG |
| dBBDu183H1579_VHR_CDR2 | 133 | QIKDKYNAYAAYYAPSVKG |
| dBBDu183H1572_VHR_CDR2 | 136 | QIKDKYNAYAAYYAPSVKG |
| dBBDu183H0883_VHR_CDR2 | 139 | QIKDKGNAYAAYYAPSVKG |
| dBBDu183H0888_VHR_CDR3 | 140 | IHYASASTLLPAFGIDA |
| dBBDu183H1595_VHR_CDR3 | 143 | IHYASASTLLPAFGVDA |
| dBBDu183H1573_VHR_CDR3 | 145 | VHYASASTLLPAFGVDA |
| dBBDu183H1579_VHR_CDR3 | 146 | VHYASASTLLPAFGVDA |
| dBBDu183H1572_VHR_CDR3 | 149 | VHYASASTLLPAEGVDA |
| dBBDu183H0883_VHR_CDR3 | 152 | VHYASASTLLPAFGVDA |

1.2 Binding Kinetics Information of Affinity Matured Variants

1.2.1 Expression and Purification of Human CD3 and CD137

The gamma and epsilon subunits of the human CD3 complex (human CD3eg linker) were linked by a 29-mer linker and a Flag-tag was fused to the C-terminal end of the gamma subunit (SEQ ID NO: 102, Tables 9 and 10). This construct was expressed transiently using FreeStyle293F cell line (Thermo Fisher). Conditioned media expressing human CD3eg linker was concentrated using a column packed with Q HP resins (GE healthcare) then applied to FLAG-tag affinity chromatography. Fractions containing human CD3eg linker were collected and subsequently subjected to a Superdex 200 gel filtration column (GE healthcare) equilibrated with 1×D-PBS. Fractions containing human CD3eg linker were then pooled. Human CD137 extracellular domain (ECD) (SEQ ID NO: 103, Tables 9 and 10) with hexahistidine (His-tag) and biotin acceptor peptide (BAP) on its C-terminus was expressed transiently using FreeStyle293F cell line (Thermo Fisher). Conditioned media expressing human CD137 ECD was applied to a HisTrap HP column (GE healthcare) and eluted with buffer containing imidazole (Nacalai). Fractions containing human CD137 ECD were collected and subsequently subjected to a Superdex 200 gel filtration column (GE healthcare) equilibrated with 1×D-PBS. Fractions containing human CD137 ECD were then pooled and stored at −80 degrees C.

TABLE 9

| Antigen name | SEQ List |
|---|---|
| Human CD3eg linker | 102 |
| Human CD137 ECD | 103 |

TABLE 10

| | SEQ | |
|---|---|---|
| Antigen name | list | Amino Acid Sequence |
| Human CD3eg | 102 | QDGNEEMGGITQTPYKVSISGTTVILTCPQYPG |
| linker | | SEILWQHNDKNIGGDEDDKNIGSDEDHLSLKEF |
| | | SELEQSGYYVCYPRGSKPEDANFYLYLRARVGS |
| | | ADDAKKDAAKKDDAKKDDAKKDGSQSIKGNHLV |
| | | KVYDYQEDGSVLLTCDAEAKNITWFKDGKMIGF |
| | | LTEDKKKWNLGSNAKDPRGMYQCKGSQNKSKPL |
| | | QVYYRMDYKDDDDK |
| Human CD137 | 103 | LQDPCSNCPAGTFCDNNRNQICSPCPPNSFSSA |
| ECD | | GGQRTCDICRQCKGVFRTRKECSSTSNAECDCT |
| | | PGFHCLGAGCSMCEQDCKQGQELTKKGCKDCCF |
| | | GTFNDQKRGICRPWTNCSLDGKSVLVNGTKERD |
| | | VVCGPSPADLSPGASSVTPPAPAREPGHSPQHH |
| | | HHHHGGGGSGLNDIFEAQKIEWHE |

1.2.2 Affinity Measurement Towards Human CD3 and CD137

Binding affinity of Dual-Fab antibodies (Dual-Ig) to human CD3 were assessed at 25 degrees C. using Biacore 8K instrument (GE Healthcare). Anti-human Fc (GE Healthcare) was immobilized onto all flow cells of a CM4 sensor chip using amine coupling kit (GE Healthcare). Antibodies were captured onto the anti-Fc sensor surfaces, then recombinant human CD3 or CD137 was injected over the flow cell. All antibodies and analytes were prepared in ACES pH 7.4 containing 20 mM ACES, 150 mM NaCl, 0.05% Tween 20, 0.005% NaN₃. Sensor surface was regenerated each cycle with 3M MgCl₂. Binding affinity was determined by processing and fitting the data to 1:1 binding model using Biacore Insight Evaluation software (GE Healthcare). CD137 binding affinity assay was conducted in same condition except assay temperature was set at 37 degrees C. Binding affinity of Dual-Fab antibodies to recombinant human CD3 and CD137 are shown in Tables 11-1 and 11-2. As illustrated in Tables 11-1 and 11-2, the DUAL Fab variants showed different binding kinetics towards CD3 and CD137 as compared H183/L072.

TABLE 11-1

| Antibody | CD3 (25° C.) | | | CD137 (37° C.) | | |
|---|---|---|---|---|---|---|
| name | ka (M−1s−1) | kd (s−1) | KD (M) | ka (M−1s−1) | kd (s−1) | KD (M) |
| H183L072 | 3.54E+04 | 1.20E−02 | 3.40E−07 | 3.47E+03 | 1.96E−02 | 5.66E−06 |
| H0868L0581 | 1.23E+05 | 1.94E−02 | 1.57E−07 | 1.22E+04 | 1.36E−03 | 1.11E−07 |
| H1550L0918 | 7.20E+04 | 3.16E−03 | 4.38E−08 | 1.09E+04 | 5.79E−03 | 5.30E−07 |
| H1571L0581 | 1.42E+05 | 1.56E−02 | 1.10E−07 | 1.21E+04 | 1.05E−03 | 8.68E−08 |
| H1610L0581 | 6.80E+04 | 1.42E−03 | 2.09E−08 | 1.07E+04 | 1.10E−03 | 1.03E−07 |
| H1610L0939 | 5.00E+04 | 2.53E−03 | 5.07E−08 | 1.30E+04 | 8.01E−04 | 6.18E−08 |
| H1643L0581 | 9.46E+04 | 2.51E−02 | 2.65E−07 | 1.23E+04 | 6.06E−04 | 4.94E−08 |
| H1644L0939 | 5.58E+04 | 8.08E−02 | 1.45E−06 | 1.21E+04 | 4.44E−04 | 3.68E−08 |
| H1647L0581 | 4.43E+04 | 1.01E−01 | 2.28E−06 | 9.98E+03 | 6.47E−04 | 6.48E−08 |
| H1649L0581 | 7.50E+04 | 3.36E−02 | 4.49E−07 | 1.29E+04 | 5.53E−04 | 4.28E−08 |
| H1649L0943 | 6.10E+04 | 4.81E−02 | 7.89E−07 | 1.43E+04 | 4.68E−04 | 3.28E−08 |
| H1651L0581 | 7.18E+04 | 3.71E−02 | 5.17E−07 | 1.40E+04 | 6.03E−04 | 4.32E−08 |
| H1652L0943 | 6.23E+04 | 6.36E−02 | 1.02E−06 | 1.29E+04 | 4.70E−04 | 3.64E−08 |
| H1673L0581 | 7.96E+04 | 1.06E−03 | 1.33E−08 | 1.19E+04 | 9.60E−04 | 8.04E−08 |
| H1673L0943 | 5.50E+04 | 1.16E−03 | 2.10E−08 | 1.22E+04 | 7.22E−04 | 5.91E−08 |
| H2591L0581 | 1.02E+05 | 5.35E−02 | 5.25E−07 | 2.04E+04 | 7.42E−04 | 3.63E−08 |
| H2594L0581 | 9.83E+04 | 5.84E−02 | 5.93E−07 | 2.09E+04 | 1.63E−03 | 7.81E−08 |

TABLE 11-2

| Antibody | CD3 (25° C.) | | | CD137 (37° C.) | | |
|---|---|---|---|---|---|---|
| Name | ka (1/Ms) | kd (1/s) | KD (M) | ka (1/Ms) | kd (1/s) | KD (M) |
| H0888L0581 | 9.50E+04 | 1.92E−03 | 2.02E−08 | 1.50E+04 | 3.11E−03 | 2.08E−07 |
| H1595L0581 | 1.16E+05 | 6.58E−03 | 5.70E−08 | 1.38E+04 | 2.69E−03 | 1.95E−07 |
| H1573L0581 | 1.21E+05 | 1.88E−02 | 1.56E−07 | 1.46E+04 | 1.03E−03 | 7.06E−08 |
| H1579L0581 | 1.24E+05 | 3.40E−02 | 2.73E−07 | 1.48E+04 | 4.06E−03 | 2.75E−07 |
| H1572L0581 | 9.77E+04 | 2.80E−02 | 2.86E−07 | 1.39E+04 | 7.22E−04 | 5.21E−08 |

| | | | | |
|---|---|---|---|---|
| H0883 | 9.07E+04 | 9.99E−03 | 1.10E−07 | n.d. |

### INDUSTRIAL APPLICABILITY

The multispecific antigen-binding molecules of the present invention are capable of modulating and/or activating an immune response while circumventing the cross-linking between different cells (e.g., different T cells) resulting from the binding of a conventional multispecific antigen-binding molecule to antigens expressed on the different cells, which is considered to be responsible for adverse reactions when the multispecific antigen-binding molecule is used as a drug.

1. A method for producing a preparation of a multispecific antigen binding molecule, wherein the multispecific antigen binding molecule comprises:
(a) a first antigen-binding moiety and a second antigen-binding moiety, each of the first antigen-binding moiety and the second antigen-binding moiety is capable of binding to a first antigen and a second antigen different from the first antigen, but does not bind both antigens at the same time; and
(b) a third antigen-binding moiety capable of binding to a third antigen different from the first and the second antigen, preferably an antigen expressed on a cancer cell/tissue, wherein each of the first antigen-binding moiety and the second antigen-binding moiety comprises at least one cysteine residue (via mutation, substitution or insertion) which is not in a hinge region, preferably said at least one cysteine locates in the CH1 region; said at least one cysteine residue is capable of forming at least one disulfide bond between the first antigen-binding moiety and the second antigen-binding moiety, preferably in the CH1 region;
wherein said method comprises contacting the preparation with a reducing reagent.

2. The method of claim 1, wherein each of the first antigen-binding moiety and the second antigen-binding moiety comprises one cysteine residue (via mutation, substitution or insertion) at position 191 according to EU numbering in the CH1 region which is capable of forming one disulfide bond between the CH1 region of the first antigen-binding moiety and the CH1 region of the second antigen-binding moiety.

3. The method of claim 2, wherein said multispecific antigen binding molecule preparation (before contacting with the reducing agent) comprises two or more structural isoforms which differ by at least one disulfide bond formed between amino acid residues located in the CH1 region or at the position 191 in the CH1 region (EU numbering), and wherein the contacting with reducing agent preferentially enriches or increases the population of a structural isoform having at least one disulfide bond formed between amino acid residues located in the CH1 region or at the position 191 in the CH1 region (EU numbering).

4. The method of any one of claims 1 to 3, wherein the pH of said reducing reagent contacting with the multispecific antigen binding molecule is from about 3 to about 10, preferably pH 6-8.

5. The method of any one of claims 1 to 4, wherein the reducing agent is selected from the group consisting of TCEP, 2-MEA, DTT, Cysteine, GSH and Na₂SO₃, preferably TCEP.

6. The method of any one of claims 1 to 5, wherein the concentration of the reducing agent is from about 0.01 mM to about 100 mM.

7. The method of any one of claims 1 to 6, wherein the concentration of the multispecific antigen binding molecule is from about 0.1 mg/ml to about 50 mg/ml, preferably about 10 mg/ml.

8. The method of any one of claims 1 to 7, further comprising a step of promoting re-oxidization of cysteine disulfide bonds, preferably by removing the reducing agent, preferably by dialysis or buffer exchange.

9. The method of any one of claims 1 to 8, wherein each of the first antigen-binding moiety and the second antigen-binding moiety is capable of binding to CD3 and CD137 but does not bind both CD3 and CD137 at the same time.

10. The method of claim 9, wherein the first antigen-binding moiety and the second antigen-binding moiety each comprises an antibody variable region comprising any one of (a1) to (a17) below:
(a1) the heavy chain complementarity determining region (CDR) 1 of SEQ ID NO: 17, the heavy chain CDR 2 of SEQ ID NO: 31, the heavy chain CDR 3 of SEQ ID NO: 45, the light chain CDR 1 of SEQ ID NO: 64, the light chain CDR 2 of SEQ ID NO: 69 and the light chain CDR 3 of SEQ ID NO: 74;
(a2) the heavy chain complementarity determining region (CDR) 1 of SEQ ID NO: 18, the heavy chain CDR 2 of SEQ ID NO: 32, the heavy chain CDR 3 of SEQ ID NO: 46, the light chain CDR 1 of SEQ ID NO: 63, the light chain CDR 2 of SEQ ID NO: 68 and the light chain CDR 3 of SEQ ID NO: 73;
(a3) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 63, the light chain CDR 2 of SEQ ID NO: 68 and the light chain CDR 3 of SEQ ID NO: 73;
(a4) the heavy chain complementarity determining region (CDR) 1 of SEQ ID NO: 19, the heavy chain CDR 2 of SEQ ID NO: 33, the heavy chain CDR 3 of SEQ ID NO: 47, the light chain CDR 1 of SEQ ID NO: 65, the light chain CDR 2 of SEQ ID NO: 70 and the light chain CDR 3 of SEQ ID NO: 75;
(a5) the heavy chain complementarity determining region (CDR) 1 of SEQ ID NO: 20, the heavy chain CDR 2 of SEQ ID NO: 34, the heavy chain CDR 3 of SEQ ID NO: 48, the light chain CDR 1 of SEQ ID NO: 63, the light chain CDR 2 of SEQ ID NO: 68 and the light chain CDR 3 of SEQ ID NO: 73;
(a6) the heavy chain complementarity determining region (CDR) 1 of SEQ ID NO: 22, the heavy chain CDR 2 of SEQ ID NO: 36, the heavy chain CDR 3 of SEQ ID NO: 50, the light chain CDR 1 of SEQ ID NO: 63, the light chain CDR 2 of SEQ ID NO: 68 and the light chain CDR 3 of SEQ ID NO: 73;
(a7) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 63, the light chain CDR 2 of SEQ ID NO: 68 and the light chain CDR 3 of SEQ ID NO: 73;
(a8) the heavy chain complementarity determining region (CDR) 1 of SEQ ID NO: 23, the heavy chain CDR 2 of SEQ ID NO: 37, the heavy chain CDR 3 of SEQ ID NO: 51, the light chain CDR 1 of SEQ ID NO: 66, the light chain CDR 2 of SEQ ID NO: 71 and the light chain CDR 3 of SEQ ID NO: 76;
(a9) the heavy chain complementarity determining region (CDR) 1 of SEQ ID NO: 24, the heavy chain CDR 2 of SEQ ID NO: 38, the heavy chain CDR 3 of SEQ ID NO: 52, the light chain CDR 1 of SEQ ID NO: 63, the light chain CDR 2 of SEQ ID NO: 68 and the light chain CDR 3 of SEQ ID NO: 73;
(a10) the heavy chain complementarity determining region (CDR) 1 of SEQ ID NO: 25, the heavy chain CDR 2 of SEQ ID NO: 39, the heavy chain CDR 3 of SEQ ID NO: 53, the light chain CDR 1 of SEQ ID NO: 66, the light chain CDR 2 of SEQ ID NO: 71 and the light chain CDR 3 of SEQ ID NO: 76;
(a11) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 66, the light chain CDR 2 of SEQ ID NO: 71 and the light chain CDR 3 of SEQ ID NO: 76;
(a12) the heavy chain complementarity determining region (CDR) 1 of SEQ ID NO: 26, the heavy chain CDR 2 of SEQ ID NO: 40, the heavy chain CDR 3 of SEQ ID NO: 54, the light chain CDR 1 of SEQ ID NO: 63, the light chain CDR 2 of SEQ ID NO: 68 and the light chain CDR 3 of SEQ ID NO: 73;
(a13) the heavy chain complementarity determining region (CDR) 1 of SEQ ID NO: 27, the heavy chain CDR 2 of SEQ ID NO: 41, the heavy chain CDR 3 of SEQ ID NO: 55, the light chain CDR 1 of SEQ ID NO: 63, the light chain CDR 2 of SEQ ID NO: 68 and the light chain CDR 3 of SEQ ID NO: 73;
(a14) the heavy chain complementarity determining region (CDR) 1 of SEQ ID NO: 28, the heavy chain CDR 2 of SEQ ID NO: 42, the heavy chain CDR 3 of SEQ ID NO: 56, the light chain CDR 1 of SEQ ID NO: 63, the light chain CDR 2 of SEQ ID NO: 68 and the light chain CDR 3 of SEQ ID NO: 73;
(a15) the heavy chain complementarity determining region (CDR) 1 of SEQ ID NO: 82, the heavy chain CDR 2 of SEQ ID NO: 83, the heavy chain CDR 3 of SEQ ID NO: 84, the light chain CDR 1 of SEQ ID NO: 65, the light chain CDR 2 of SEQ ID NO: 70 and the light chain CDR 3 of SEQ ID NO: 75;
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

11. The method of claim 10, wherein the first antigen-binding moiety and the second antigen-binding moiety each comprises an antibody variable region comprising any one of (a1) to (a17) below:
(a1) a heavy chain variable region comprising an amino acid sequence of SEQ ID NO: 3, and a light chain variable region comprising an amino acid sequence of SEQ ID NO: 59;
(a2) a heavy chain variable region comprising an amino acid sequence of SEQ ID NO: 4, and a light chain variable region comprising an amino acid sequence of SEQ ID NO: 58;
(a3) a heavy chain variable region comprising an amino acid sequence of SEQ ID NO: 5, and a light chain variable region comprising an amino acid sequence of SEQ ID NO: 58;
(a4) a heavy chain variable region comprising an amino acid sequence of SEQ ID NO: 5, and a light chain variable region comprising an amino acid sequence of SEQ ID NO: 60;
(a5) a heavy chain variable region comprising an amino acid sequence of SEQ ID NO: 6, and a light chain variable region comprising an amino acid sequence of SEQ ID NO: 58;
(a6) a heavy chain variable region comprising an amino acid sequence of SEQ ID NO: 8, and a light chain variable region comprising an amino acid sequence of SEQ ID NO: 58;
(a7) a heavy chain variable region comprising an amino acid sequence of SEQ ID NO: 9, and a light chain variable region comprising an amino acid sequence of SEQ ID NO: 58;
(a8) a heavy chain variable region comprising an amino acid sequence of SEQ ID NO: 9, and a light chain variable region comprising an amino acid sequence of SEQ ID NO: 61;
(a9) a heavy chain variable region comprising an amino acid sequence of SEQ ID NO: 10, and a light chain variable region comprising an amino acid sequence of SEQ ID NO: 58;
(a10) a heavy chain variable region comprising an amino acid sequence of SEQ ID NO: 11, and a light chain variable region comprising an amino acid sequence of SEQ ID NO: 61;
(a11) a heavy chain variable region comprising an amino acid sequence of SEQ ID NO: 12, and a light chain variable region comprising an amino acid sequence of SEQ ID NO: 61;
(a12) a heavy chain variable region comprising an amino acid sequence of SEQ ID NO: 12, and a light chain variable region comprising an amino acid sequence of SEQ ID NO: 58;
(a13) a heavy chain variable region comprising an amino acid sequence of SEQ ID NO: 13, and a light chain variable region comprising an amino acid sequence of SEQ ID NO: 58;
(a14) a heavy chain variable region comprising an amino acid sequence of SEQ ID NO: 14, and a light chain variable region comprising an amino acid sequence of SEQ ID NO: 58; and
(a15) a heavy chain variable region comprising an amino acid sequence of SEQ ID NO: 81, and a light chain variable region comprising an amino acid sequence of SEQ ID NO: 60.
(a16) an antibody variable region that binds to the same epitope of any of the antibody variable region selected from (a1) to (a15); and
(a17) an antibody variable fragment that competes with the binding of any of the antibody variable fragment selected from (a1) to (a15).

12. The method of any one of claims 1 to 11, wherein the third antigen-binding moiety is capable of binding to DLL3, preferably human DLL3.

13. The method of claim 12, wherein the third antigen-binding moiety capable of binding to DLL3 comprises an antibody variable region comprising the heavy chain complementarity determining region (CDR) 1 of SEQ ID NO: 233, the heavy chain CDR 2 of SEQ ID NO: 234, the heavy chain CDR 3 of SEQ ID NO: 235, the light chain CDR 1 of SEQ ID NO: 237, the light chain CDR 2 of SEQ ID NO: 238 and the light chain CDR 3 of SEQ ID NO: 239

14. The method of claim 13, wherein the third antigen-binding moiety capable of binding to DLL3 comprises an antibody variable region comprising: a heavy chain variable region comprising an amino acid sequence of SEQ ID NO: 232, and a light chain variable region comprising an amino acid sequence of SEQ ID NO: 236.

15. The method of any one of claims 1 to 14, wherein the multispecific antigen binding molecule further comprises a Fc domain.

16. A preparation of a multispecific antigen binding molecule prepared according to the method of any one of claims 1 to 15, said preparation having a homogeneous population of said multispecific antigen binding molecule having at least one disulfide bond in the CH1 region (position 191 according to EU numbering).

17. A preparation of a multispecific antigen binding molecule prepared according to the method of any one of claims 1 to 15, said preparation having at least 80%, 90%, preferably at least 95% molar ratio of said multispecific antigen binding molecule having at least one disulfide bond in the CH1 region (position 191 according to EU numbering).
